(19) United States
(12) Patent Application Publication
(10) Pub. No.: US 2025/0134928 A1
(43) Pub. Date: May 1, 2025

(54) METHODS FOR IDENTIFYING AND USING ALLOGENEIC TUMOR INFILTRATING LYMPHOCYTES TO TREAT CANCER

(71) Applicant: OBSIDIAN THERAPEUTICS, INC., Cambridge, MA (US)

(72) Inventors: Jan ter Meulen, Cambridge, MA (US); Scott Lajoie, Cambridge, MA (US); Scott Francis Heller, Stoughton, MA (US); Jeremy Hatem Tchaicha, Belmont, MA (US); Rachel Burga, Cambridge, MA (US); Mithun Khattar, Cambridge, MA (US); Kyle Pedro, Cambridge, MA (US); Paul K. Wotton, Cambridge, MA (US)

(73) Assignee: OBSIDIAN THERAPEUTICS, INC., Cambridge, MA (US)

(21) Appl. No.: 18/723,120

(22) PCT Filed: Jan. 18, 2023
(86) PCT No.: PCT/US2023/060793

§ 371 Date: Jun. 21, 2024

Related U.S. Application Data
(60) Provisional application No. 63/266,892, filed on Jan. 18, 2022.

(51) Int. Cl. A61K 35/17; A01K 67/0276; A61K 40/11; A61K 40/42; A61P 35/00; C07K 14/54; C12N 5/0783; G01N 33/50; G01N 33/569; G01N 33/68

(52) U.S. Cl. CPC A61K 35/17; A01K 67/0276; A61K 40/11; A61K 40/4234; A61P 35/00; C07K 14/5443; C12N 5/0636; G01N 33/5011; G01N 33/5044; G01N 33/56972; G01N 33/68; A01K 2207/15; A01K 2227/105; A01K 2267/0331; C12N 2502/30; G01N 2333/96416

(57) ABSTRACT

Provided herein are allogenic tumor-infiltrating lymphocytes (TILs) engineered to express a membrane-bound interleukin 15 (mbIL 15). The allogeneic mbIL 15 TILs can be expanded in vitro using a rapid expansion protocol without the use of exogenous interleukin 2 (IL2) and can be used in allogeneic adoptive cell therapy without concomitant use of an exogenous cytokine such as IL2. The allogeneic TIL can be further engineered such that the mbIL 15 is operably linked to one or more drug responsive domains (DRDs), polypeptides that can regulate the abundance and/or activity of the IL15 upon binding of the DRD with a ligand. Also provided methods of screening for efficacy of allogeneic TILs using an allogeneic patient derived xenograft.

### CROSS REFERENCE TO RELATED APPLICATION

This application claims priority to U.S. Provisional Application No. 63/266,892, filed Jan. 18, 2022, which is incorporated herein in its entirety.

### REFERENCE TO THE SEQUENCE LISTING

The instant application contains a Sequence Listing which has been submitted electronically in XML format and is hereby incorporated by reference in its entirety. Said Sequence Listing XML, created on Jan. 18, 2023, is named 108407-1360072-Jan-18.xml and is 120,868 bytes in size.

### BACKGROUND

Solid tumors present major challenges in the development of effective adoptive cell therapies (ACTs). For example, targeting a single tumor antigen can lead to antigen loss or recurrence of more aggressive clones. Additionally, infiltration of the therapeutic cells into a solid tumor can prove challenging and, even if the cells infiltrate the tumor, the tumor microenvironment can be inhospitable due to immune suppressive mechanisms. ACT with tumor-infiltrating lymphocytes (TILs) has been proposed as a treatment modality that addresses these issues, at least for certain solid tumor. TILs, for example, include T cells with multiple T cell receptor (TCR) clones and are thus better able to recognize multiple tumor antigens and thereby address tumor heterogeneity. Additionally, TILs recognize specific tumor antigens, allowing them to target tumors, which are antigenically distinct from surrounding healthy tissue.

Typically, TILs that recognize tumor antigens are prepared from a tumor site of a subject using a tumor biopsy or a sample of a surgically removed tumor. The TILs are then stimulated and expanded in vitro in the presence of stimulators, such as interleukin-2 (IL2) and feeder cells, like peripheral blood mononuclear cells (PBMCs). After expansion, the TILs are then infused back into the patient with concurrent administration of IL2. The IL2, however, is highly cytotoxic and is not well tolerated by patients, making many ineligible for TIL therapy. Additionally, in some cases, however, TILs cannot be derived from the same patient in need of treatment. Improvements in the field are needed to make ACT using TILs a more accessible and effective treatment for cancer.

### SUMMARY

This disclosure relates to a method for treating cancer in a recipient subject by administering to the recipient subject a population of allogeneic tumor-infiltrating lymphocytes (TILs). Optionally, the method does not require concomitant IL2 administration. The allogeneic TILs are optionally modified to express a membrane bound IL15 (mbIL15). The methods described herein provide the option of TIL therapy for subjects who might not otherwise be eligible, for example, subjects from who sufficient TILs cannot be prepared for autologous implantation or for whom the time required to expand autologous TILs is simply not available.

The allogeneic TILs from a donor can be partially or fully HLA-matched (Human Leukocyte Antigen-matched) with the recipient subject and/or can be matched for common antigens with the recipient tumor cells. Thus the method can further comprise selecting allogenic TILs from an HLA-matched donor or selecting allogenic TILs with common antigens.

The TIL can optionally be expanded in vitro or in vivo in the absence of an exogenous cytokine like interleukin 2 (IL2). Systemic administration of IL2 to cancer patients concomitant with or following TIL immunotherapy often causes toxicity in patients who are already medically fragile. Many patients suffer severe, life-threatening side effects after IL2 administration, including hypotension and shock due to capillary leakage syndrome. TIL therapy with low doses of concomitant IL2 was less effective than at higher doses. Thus the modified TIL described herein can be used in a treatment regimen that is less toxic to a subject with cancer than current treatment regimens that require the use of exogenous IL2.

The TIL can be further engineered such that the mbIL15 is operably linked to one or more drug responsive domains (DRDs). The DRDs are polypeptides that can regulate the abundance and/or activity of a payload, such as mbIL15, upon binding with a ligand. Multiple DRDs, for example, in series, can regulate a single payload. The one or more DRDs are operably linked to the mbIL15 such that interaction of the DRD with an effective amount of ligand under appropriate conditions results in modifying the biological activity of the payload.

Also provided is a population of modified TILs. The plurality of TILs optionally includes a subpopulation of modified TILs that has undergone expansion. Thus, also provided herein is an expanded TIL engineered to express a mbIL15, optionally operably linked to a DRD. A population of expanded TILs is also disclosed herein. Following expansion, the population of TILs survives more than 5 days, more than 10 days, or more than 15 days in a culture lacking feeder cells, even in the absence of exogenous cytokines. Similarly, the population of TILs survives in vivo without exogenous cytokine administration.

The population of expanded TILs has a greater proportion of CD8+ cells and a lower proportion of CD4+ cells as compared to the proportion of CD8+ cells and CD4+ cells in a control population of unexpanded TILs. Thus, the population of expanded TILs has a CD4:CD8 ratio lower than the CD4:CD8 ratio of a control population of unexpanded TILs. Additionally, the population of expanded mbIL15 TILs has a lesser proportion of CD4 T_{reg} cells as compared to the proportion of CD4 T_{reg} cells in the pre-REP TILs prior to engineering and expansion in REP. The population of expanded TILs also has a lesser proportion of PD1+ cells as compared to the proportion of PD1+ cells in a control population of unexpanded TILs. The population of expanded TILs as described herein also has a greater proportion of cells producing both tumor necrosis factor α (TNFα) and producing interferon γ (IFNγ) as compared to the proportion of TILs producing both tumor necrosis factor α (TNFα) and interferon γ (IFNγ) in a control population of unexpanded TILs.

Also described herein is a mixed population of allogeneic TILs that includes a subpopulation of unmodified TILs, and a subpopulation of modified TILs comprising mbIL15, which is, optionally, operably linked to a DRD. The subpopulation of modified TILs expands in the presence of K562 feeder cells, 41BB ligand (41BBL), and interleukin 21 (IL21, secreted or membrane bound to the K562 feeder cells) and expands more than the subpopulation of unengineered (i.e., unmodified) TILs in the presence of K562 feeder cells, 41BBL, and IL21. This preferential expansion of the subpopulation of engineered (i.e., modified) TILs occurs in the absence of exogenous cytokines, like IL2.

A method of making allogeneic TILs engineered to express mbIL15 includes transducing the donor TIL with a vector, wherein the vector comprises a first nucleic acid sequence that encodes IL15 and a second nucleic acid sequence that encodes a transmembrane domain. The vector used to transduce the TIL can be a viral vector, such as a gamma-retroviral vector or a lentiviral vector, more particularly, a gibbon ape leukemia virus (GALV) pseudotyped gamma-retroviral vector or a baboon endogenous retrovirus envelope (BaEV) pseudotyped lentiviral vector. Thus, provided herein is a GALV pseudotyped retroviral vector or a BaEV pseudotyped lentiviral vector comprising a first nucleic acid sequence that encodes IL15 and a second nucleic acid sequence that encodes a transmembrane domain. Upon expression of the first and second nucleic acid sequences, the transmembrane domain serves to anchor the IL15 to or within the cell membrane, optionally linked to the IL15 via a linker or a hinge.

Also provided is a pharmaceutical composition comprising any allogenic TIL or population of allogeneic TILs described herein and a pharmaceutical carrier. Any allogeneic TIL, any population of allogeneic TILs, or any pharmaceutical composition thereof can be administered to a recipient subject with cancer as a method of treating cancer. The method optionally further comprises administering to the recipient subject a second agent, wherein the second agent is a ligand that binds to a DRD operably linked to mbIL15. Upon administration of an effective amount of the ligand and binding of the ligand to the DRD, the biological activity of the mbIL15 is increased in the subject. The treatment method with or without a DRD operably linked to the mbIL15, does not require that the subject be administered an exogenous cytokine, such as IL2.

The treatment method optionally includes isolating one or more TILs from a donor tumor and introducing into the one or more TILs a nucleic acid sequence that expresses mbIL15. The donor subject is not the recipient subject. TILs prepared from the tumor of the donor subject can be selected such that the TILs prepared from the donor comprise one or more t-cell receptors (TCR) that are specific for the cancer antigens are present in the tumor of the recipient subject. Optionally, the method further comprises selecting a donor subject that is HLA-matched to the recipient subject. In the treatment methods described herein, the recipient subject is lymphodepleted prior to administration of the TILs.

Also provided herein is a method for assessing the efficacy of the allogeneic TILs by implanting tumor cells or fragments from the recipient subject into an immunodeficient or humanized starter animal under conditions for growth of a starter tumor; implanting tumor cells or fragments from the starter tumor to an immunodeficient or humanized test animal to create a test tumor; administering to the test animal the allogeneic TILs; and evaluating the allogeneic TIL efficacy in treating the test tumor. The allogeneic TIL efficacy can be evaluated, for example, by measuring the size of the test tumor before and after administration of the allogeneic TILs to the test animal, by detecting TIL infiltration into the test tumor after administration, or by imaging of the test animal over the course of treatment. The starter and test animals can be mice such as humanized mice, athymic nude mice, severely compromised immune deficient (SCID) mice, NOD-SCID mice, or recombination-activating gene 2 (Rag2)-knockout mice. Because the allogenic TILs are modified to express mbIL15, the TILs can be administered to the starter animal and the test animal without the need for exogenous cytokines such as IL2.

Thus, the allogenic TILs are optionally derived from a donor and optionally modified to express mbIL15. The optionally modified TILs can be expanded in vitro, without the need for exogenous cytokine such as IL2. The autologous TILs, optionally modified to express mbIL15, can be expanded in the presence of K562 feeder cells, wherein the K562 feeder cells are modified to express 41BB ligand (41BBL) and IL21 (e.g., mbIL21).

The identified embodiments are exemplary only and are therefore non-limiting. The details of one or more non-limiting embodiments of the invention are set forth in the accompanying drawing and the description below. Other embodiments of the invention should be apparent to those of ordinary skill in the art after consideration of the present disclosure.

### BRIEF DESCRIPTION OF THE DRAWINGS

FIG. 1 shows frequency of CD45+ cells (left) and CD3+ T cells within CD45+ cells (right) in fresh tumor digest and after 3 weeks pre-REP TIL culture.

FIG. 2 shows transduction efficiency of IL15-293 construct in two melanoma TIL donors measured by flow cytometry on day 5 post-transduction.

FIG. 3A-3B show antigen and IL2-independent expansion and survival of TILs expressing mbIL15. FIG. 3A shows TIL donor 006 cells (TIL 006) transduced with constitutive mbIL15 or GFP and expanded in REP for 12 days with or without 6000 IU/mL IL2. FIG. 3B shows TIL 006 transduced with constitutive mbIL15 (expanded in REP without IL2) or GFP (expanded in REP with 6000 IU/mL IL2) and enumerated in a 14-day antigen-independent survival assay, with and without 6000 IU/mL IL2.

FIG. 4 shows antigen-independent TIL expansion after a rapid expansion protocol (REP). After REP, unengineered and mbIL15 engineered TILs (constitutive or regulated mbIL15) were plated with or without exogenous IL2 or acetazolamide (ACZ), and new wells were harvested every 3 days to assess cell enumeration and phenotype.

FIG. 5 shows TIL expansion in an antigen-dependent setting. After a rapid expansion protocol (REP), unengineered and mbIL15 engineered TILs were plated with HLA-matched mitomycin C-treated melanoma cells in a TIL:tumor co-culture assay with and without exogenous IL2, acetazolamide, or vehicle (DMSO) and wells were harvested every 3 days to assess cell enumeration and phenotype.

FIG. 6A-B show tumor reactivity of TILs after a rapid expansion protocol (REP). FIG. 6A shows TIL 006 and TIL 005, both transduced with regulated mbIL15 and unengineered controls and co-cultured for 24-hours with HLA-matched mitomycin-C treated melanoma cells. IFNγ in supernatants was measured by MSD assay. FIG. 6B shows cytotoxicity of TILs in co-culture as measured by loss of luminescence by luciferase-tagged HLA-matched melanoma line.

FIG. 7A-B show TIL expansion and transduction efficiency prior to infusion into animals for an in vivo adoptive cell therapy experiment. FIG. 7A shows cell expansion for TIL donor 006, used for in vivo adoptive cell transfer (ACT), of unengineered and mbIL15 engineered TILs. FIG. 7B shows transduction efficiency after a rapid expansion protocol (REP); unengineered and mbIL15 engineered TILs were assessed for expression of IL15 and IL15RaFc as a measure of transduction efficiency.

FIG. 8A-C show analyses of TIL enumeration and IL15 expression for in vivo adoptive cell therapy experiment. FIG. 8A shows enumeration of adoptively transferred unengineered and mbIL15 engineered TILs by flow cytometry from peripheral blood samples. TILs were identified as live humanCD3+murineCD45− cells in submandibular vein blood samples. FIG. 8B and FIG. 8C show TIL enumeration (hCD3+mCD45−) and IL15 expression (IL15+IL15RaFc+) of splenic and bone marrow samples isolated 14 days or 53 days after ACT.

FIG. 9 shows acetazolamide (ACZ) regulation of IL15 expression and signaling in cryopreserved regulated mbIL15 TILs occurs in a dose-dependent fashion. Regulated mbIL15 TILs from four patients (Patients 1-4) were thawed and rested in ACZ-free media for 24 hours, then regulated in 0.1, 1, 2.5, 5, 10, 25, 100 μM of ACZ for 18 hours. Regulated mbIL15 TILs were then collected and analyzed for IL15 expression and signaling using a phospho-flow cytometry-based assay. FIG. 9A shows the frequency of IL15+TILs as a percentage of CD3+ cells. FIGS. 9B-9E show the results for each patient: here, cells were further gated on IL15+, and then geometric mean fluorescent intensity for each pSTAT5 (open square) and pS6 (closed circle) was calculated. Values shown are set relative to vehicle control. N=4 human donors.

FIG. 10 shows the mean fluorescence intensity (MFI) for pSTAT5 and pS6 in patients 1-4. FIG. 10A shows the MFI for pSTAT5. FIG. 10B shows the MFI for pS6.

FIG. 11 shows constitutive mbIL15 expression and ACZ regulation of regulated mbIL15 TILs engage the IL15 signaling pathway. Here, unengineered TILs and regulated mbIL15 TILs from Patients 1-3, were thawed and rested in ACZ-free media for 24 hours, then regulated with IL2 or ACZ for 18 hours. Cells were then collected and analyzed for IL15 expression and signaling using a phospho-flow cytometry-based assay. Unengineered TILs and regulated mbIL15 TILs+ vehicle were gated on Live cells followed by singlets, followed by CD3+. Constitutive IL15 TILs and regulated mbIL15 TILs+ ACZ conditions were further gated on IL15+ staining. Geometric mean fluorescent intensity for each pSTAT5 and pS6 was calculated. N=3 human donors.

FIG. 12 shows regulated mbIL5-modified TILs without exogenous cytokines demonstrate greater polyfunctionality than unengineered TILs+IL2. Unengineered TILs and regulated mbIL15 TILs were thawed and rested in ACZ-free media for 24 hours; next, the unengineered TILs were treated with the following concentrations of IL2: 20, 200, 1000 and 6000 IU/mL, or vehicle; and regulated mbIL15 TILs were treated with the following concentrations of ACZ: 0.1, 1, 5, 10, 25, 100 μM ACZ, or vehicle. Treatments were for 18 hours. Cells were stimulated with PMA and ionomycin for 6 hours in the presence of brefeldin A and monensin. Unstimulated TILs were used as controls (data not shown). After stimulation, cells were analyzed for expression of IL15 and intracellular TNFα and IFNγ using a flow cytometry-based assay. TILs were gated on Live cells, followed by singlets, followed by CD3+, and regulated mbIL15 TILs were additionally gated on IL15+. FIG. 12A shows TNFα and IFNγ double positive populations for unengineered TILs with IL2, and regulated mbIL15 TILs with ACZ. FIG. 12B shows IL15 expression in regulated mbIL15 TILs cultures. FIG. 12C shows a comparison of select IL2 (200 IU/mL) and ACZ (25 μM) doses.

FIG. 13 shows the results of a patient-derived xenograft (PDX) efficacy model. At the end of the end of the rapid expansion protocol (REP), unengineered TILs and regulated mbIL15 TILs (+/−acetazolamide (ACZ)) were adoptively transferred into mice bearing a human melanoma PDX. Mean tumor volumes were evaluated (+/−SEM). FIG. 13A shows mean tumor volume for a given treatment at days post adoptive cell transfer (ACT). FIG. 13B shows tumor volume at days post ACT for no TILs (top left); unengineered TILs-IL2 (top right); regulated mbIL15 TILs+vehicle (bottom left); and regulated mbIL15 TILs+ACZ (bottom right). Here, regulated mbIL15 TILs+ACZ significantly superior anti-tumor efficacy compared to unengineered TIL+IL2 (*p<0.05; Mann U Whitney).

FIG. 14 shows the results of a SK-MEL-1 xenograft cancer model. At the end of the end of the rapid expansion protocol (REP), unengineered TILs and regulated mbIL15 TILs (+/−acetazolamide (ACZ)) were adoptively transferred into mice bearing SK-MEL-1 tumors. Mean tumor volumes were evaluated (+/−SEM). FIG. 14A shows mean tumor volume for a given treatment at days post adoptive cell transfer (ACT). FIG. 14B shows tumor volume at days post ACT for no TILs (top left); unengineered TILs+IL2 (top right); regulated mbIL15 TILs+vehicle (bottom left); and regulated mbIL15 TILs+ACZ (bottom right). Here, regulated mbIL15 TILs+ACZ show significantly superior anti-tumor efficacy compared to unengineered TIL+IL2 (*p<0.05; Mann U Whitney).

FIG. 15 shows regulated mbIL15 TILs achieve enhanced MHC-I-dependent cytotoxicity against melanoma in vitro. Here, unengineered TILs and regulated mbIL15 TILs were cryopreserved at the end of the rapid expansion protocol (REP). Cryopreserved TILs were thawed and rested in cytokine-free conditions overnight, and then co-cultured with Cell Trace Violet-labeled melanoma cells (SK-MEL-1) at a 1:1 and 5:1 effector-to-target (TIL:melanoma) ratios. To control for MHC-1 dependent cytotoxicity, melanoma cells were pre-treated with 80 μg/mL HLA ABC MHC blocking antibody for 2 hours prior to the assay. After 3 hours of co-culture, the SK-MEL-1 cells were evaluated for expression of intracellular cleaved-caspase 3 (a marker for irreversible commitment to cell death) by flow cytometry. Quantified cleaved caspase 3 was normalized to that of target cells alone (spontaneous or background release). Bar graphs show expression of cleaved caspase-3 on target tumor cells when co-cultured with TILs from 6 individual patients.

FIG. 16 is a graph showing that maximal TIL expansion in REP occurs when mbIL15 TILs (constitutive) are generated with K562 feeder cells with both IL21 and 41BBL-mediated co-stimulation.

FIG. 17 is a graph showing that maximal TIL expansion in REP occurs when unengineered TILs are generated with pooled PBMC feeders or K562 feeder cells expressing membrane-bound IL21 and 41BBL.

FIG. 18 shows that maximal expansion of IL15-TILs in REP occurs when TILs with mbIL15 (constitutive) are generated with K562 feeder cells and receiving both IL21 and 41BBL-mediated co-stimulation. Results on feeder cells at days 8, 11,15, and 18 are shown from left to right: PBMC feeders, K562-parental feeders, K562+41BBL, K562+41BBL feeders with recombinant human IL21, K562+mbIL21 feeders, K562+41BBL+mbIL21 feeders.

FIG. 19 is a graph showing that IL15 expression is enriched through the REP process in mbIL15 TILs (constitutive) generated with K562 feeder cells and receiving both IL21 and 41BBL-mediated co-stimulation.

FIG. 20 is a graph showing expanded TILs with mbIL15 generated with K562 feeder cells with both IL21 and 41BBL-mediated co-stimulation have a decreased CD4:CD8 ratio throughout REP. Thus, TILs with mbIL15 expanded in the presence of K562 feeder cells with both IL21 and 41BBL stimulation are enriched for CD8+ cytotoxic effector cells, in contrast to expanded TILs with mbIL15 generated with pooled PBMC feeders, unmodified K562 feeders, or K562 feeders expressing 41BBL in the absence of IL21. CD4:CD8 ratios are shown at days 8, 11, 15, and 18 from left to right: PBMC feeders, K562-parental feeders, K562+41BBL, K562+41BBL feeders with recombinant human IL21, K562+mbIL21 feeders, K562+41BBL+mbIL21 feeders.

FIG. 21 is a graph showing a higher percentage of TNFα+interferon γ+ cells in expanded mbIL15 TILs generated with K562 feeder cells expressing both mbIL21 and 41BBL, as compared to mbIL15 TILs generated with PBMC feeder cells or unmodified K562 feeder cells. The higher percentage of TNFα+interferon γ+TILs is indicative of enhanced polyfunctionality in expanded mbIL15 TILs generated with K562 feeder cells expressing both mbIL21 and 41BBL.

FIG. 22 is a graph showing the results of a 10-day survival assay for mbIL15 TILs generated with PBMC feeder cells, unmodified K562 feeder cells, K562 feeder cells expressing only mb41BBL, K562 feeder cells expressing only mbIL21, K562 feeder cells expressing both 41BBL and mbIL21, and K562 feeder cells expressing 41BBL in the presence of recombinant human IL21. Expanded mbIL15TILs generated with K562 feeder cells and receiving both IL21 and 41BBL-mediated co-stimulation demonstrated improved post-REP antigen-independent survival as compared to mbIL15 TILs generated with PBMC feeder cells or K562 feeder cells that are unmodified or modified to express mbIL21 or 41BBL independently.

FIG. 23 shows the relative proportion of TCRVβ subfamilies in unengineered TILs and mbIL15 TILs expanded under with PBMC feeders, K562 feeders, K562+mbIL21 feeders, K562+41BBL feeders, K562+41BBL+mbIL21 feeders, or K562+41BBL+rhIL21 feeders. Expanded mbIL15 TILs and unengineered TILs maintain diverse subfamily distribution regardless of feeder cells or conditions.

FIG. 24 shows the expression of PD1 on the surface of mbIL15 TIL, as gated on live CD3+ cells from left to right in unexpanded TIL, and expanded TIL generated with PBMC feeders, K562-parental feeders, K562+41BBL feeders, K562+41BBL feeders with recombinant human IL21, K562+mbIL21 feeders, and K562+41BBL+mbIL21 feeders. PD1 expression is highest in unexpanded mbIL15 TIL, and expansion of mbIL15 TILs with both 41BBL and IL21-mediated signaling produces TILs with near baseline expression of PD1.

FIG. 25 shows phenotyping comparing pre-REP TILs (as described in Example 1) to engineered mbIL15 TILs (as described in Example 3). Pre-REP and post-REP TILs were phenotyped by flow cytometry using antibodies for CD3, CD4, CD8, and PD1 as described in Example 13. As shown in FIG. 25A, the frequency of CD8+ T cells is higher and the frequency of CD4+ T cells is lower for post-REP mbIL15 TILs as compared with corresponding pre-REP TILs from the same TIL donors. In FIG. 25B, the post-REP mbIL15 TILs express lower levels of PD1 than corresponding pre-REP TILs from the same TIL donors. FIG. 25C shows the percentages of a regulatory T cell population in mbIL15 TIL, identified as CD3+ T cells that are gated as CD4+ and further classified as CD25 and FoxP3 double positive cells. mbIL15 TILs have a reduced proportion of regulatory T cells as compared to preREP TILs prior to the engineering step.

FIG. 26 shows the expression of conserved melanoma-associated antigens MART-1 and gp100 on the A375 melanoma cell line and on patient-derived xenograft (PDX) cells (PDX 163A, described in Example 11), as determined by flow cytometry.

FIG. 27 shows the percentage of MART-1-tetramer positive TILs and gp 100-tetramer positive TILs in mbIL15 TIL derived from four distinct TIL donors that are HLA-matched to PDX 163A. The tetramer positive populations indicate that the TILs contain a portion of cells that are reactive to the corresponding melanoma-associated antigens, through the HLA:A2:01 locus. Donors indicated with a * were utilized in the PDX efficacy study as depicted in FIG. 30.

FIG. 28 shows interferon gamma (IFNγ) production after TIL:tumor cell co-culture to accurately predict TIL donors that are reactive to the PDX. This in vitro assay demonstrates that TIL donors 006, 39A, and 41A are the donors with the highest amount of IFNγ produced in response to the PDX, thus supporting their candidacy as donors to examine in vivo efficacy as described in Example 15.

FIG. 29 is a schematic showing an exemplary melanoma patient-derived xenograft model treated with expanded TILs that express mbIL15 operably linked to a CA2 DRD and the CA2 ligand ACZ.

FIG. 30 shows that treatment of patient-derived xenograft models according to the treatment paradigm shown in FIG. 29 results in superior anti-tumor efficacy as compared to treatment with an unengineered TIL and concomitant IL2 treatment. At the end of the end of the rapid expansion protocol (REP), unengineered TILs and regulated mbIL15 TILs (+/−acetazolamide (ACZ)) were adoptively transferred into mice bearing a human melanoma PDX. Mean tumor volumes were evaluated (+/−SEM).

FIG. 31A-B shows that TILs express mbIL15 operably linked to a CA2 DRD show significantly more intratumoral infiltration than unengineered TILs+IL2. FIG. 31A are photomicrographs of tumor sections stained immunohistochemically for human CD3 and showing intratumoral infiltration of TILs in animals treated with unengineered TILs and IL2, animals treated with TILs expressing mbIL15 operably linked to a CA2 DRD in the presence and absence of the CA2 ligand ACZ. FIG. 31B are graphs showing TIL numbers in stroma+tumor, stroma only, and tumor only.

### DETAILED DESCRIPTION

Current processes for TIL therapy require isolating a tumor sample from a subject in order to derive TILs from the tumor to be treated. Current dogma is that only TILs from the same tumor (autologous TILs) are sufficiently antigen matched to attack the tumor cells in a given patient. However, the present description provides allogeneic TILs from a donor that can be used to treat cancer in a recipient subject, wherein the donor and recipient are not the same subject, and provides methods of screening for efficacy of allogeneic TILs.

### Selecting an Allogeneic TIL

The allogeneic TILs are optionally from a donor HLA-matched with the recipient subject. Processes for HLA matching are known in the art and can be performed by a person skilled in the art, using current criteria for the selection of unrelated donors for hematopoietic cell transplantation (HCT), based on high resolution definition of the class I HLA-ABC, and class II DRB1, DQB1 alleles. See Petersdorf (2008) Optimal HLA matching in hematopoietic cell transplantation, Curr. Opin. Immunol. 20:588-593.

HLA plays an important role in allogeneic transplantation and the degree of HLA match or mismatch between the recipient and donor directly affect the survival of a transplant recipient. The HLA of a potential donors can be genotyped to select the best possible match. One or more HLA can be matched, for example, HLA-A, HLA-B, HLA-C, DRB1, DQB1. For purposes of HLA matching a TIL from a donor subject with a recipient subject, according to the present methods, similar assessments and matching can be performed. For example, a tissue sample or a TIL from a donor subject can be genotyped. TILs from the donor are optionally frozen for later use for treating one or more partially or completely HLA matched recipient subjects whose tissue samples has been HLA genotyped.

Additionally, or even in the absence of HLA matching, TILs from the donor can be matched for reactivity to certain antigens in the tumor cells of the recipient subject. Thus, provided is optimizing the efficacy of an allogeneic TIL preparation for a recipient by (a) determining the expression of shared tumor antigens (e.g., cancer-testis antigens such as MAGE-A family antigens, or differentiation antigens such as MART1 and gp 100) in the tumor(s) of the prospective TIL recipient by methods used by a person skilled in the art, such a gene expression profiling or immunostaining of tumor biopsies: (b) determining within the TIL preparation the proportion of T-cells with T-cell receptors (TCR) specific for the shared tumor antigens expressed by tumor(s) of recipient, as identified by methods used by a person skilled in the art, such as tetramer staining, ELISPOT assay, or TCR repertoire sequencing; and (c) selecting an HLA-matched, allogeneic TIL preparation with an acceptable match of TCRs specific for shared tumor antigens and expression of these antigens in the prospective TIL recipient.

Common antigens are described, for example, in Cheever et al. (2009) The Prioritization of Cancer Antigens: A National Cancer Institute Pilot Project for the Acceleration of Translational Research, Human Cancer Biology 15 (17): 5323-5337. To determine matching of common antigens, antigens expressed in the tumor of the prospective TIL recipient are identified and the TCR-specificity of the donor TILs for these antigens is assessed. The higher the “donor TIL-TCR/recipient tumor antigen” match, the greater the potency against the recipient tumor. This efficacy can be determined using an allogeneic PDX model as described herein. By way of example, TILs from a donor with 10-100% TCR-specificity for MART1 or other antigens can be selected, whereas TIL from a donor with less than 10% or less than 1% MART1 or other antigens TCR-specificity would be rejected. One or more antigens can be determined, for example, 1, 2, 3, 4, 5, 6, 7, 8, 9, or 10 antigens can be selected for screening of antigen specificity.

Also provided herein is a method of assessing the potential efficacy of an allogeneic TIL preparation for a recipient by (a) determining the cytotoxic effect of the TIL preparation on an HLA-matched tumor cell line expressing shared tumor antigens, for example, by measuring the induction of caspase 3 cleavage in the tumor cells by flow cytometry.

### Modified Tumor Infiltrating Lymphocytes (TILs)

The TILs described herein have unique properties that make them particularly potent for allogeneic use. Current processes for expanding TILs requires an interleukin 2 (IL2)-based TIL expansion (pre-rapid expansion protocol or pre-REP) followed by a rapid expansion protocol (REP). During the pre-REP stage, TILs are cultured with exogenous IL2 and the presence of tumor antigens in the chunks of dissected tumor tissue. Thus, pre-REP requires IL2 in the absence of feeder cells. The REP step typically requires added feeder cells to support rapid TIL expansion. REP feeder cell and TIL stimulation are typically irradiated peripheral blood mononuclear cells (PBMCs) and high doses of IL2 and, optionally, anti-CD3 antibody (OKT3). The IL2 during REP, however, tends to exhaust the TILs, resulting in a less potent TIL product. After in vitro REP using the current processes, expanded TILs are administered to the patient along with IL2, which may be given before, during, and/or after TIL administration, again pushing the TILs to exhaustion. The current general protocol for TIL therapy requires high-dose IL2 administration beginning on the same day or the day after TIL infusion. By way of example, a high-dose IL2 regimen can consist of bolus intravenous infusions every eight hours until tolerance, for a maximum of 14 doses, nine days of rest, and a repeat for another 14 doses. Other IL2 regimens may consist of a four-day cycle of IL2 administration that is repeated every 28 days for a maximum of four cycles or a PEGylated IL2 regimen that lasts up to 21 days.

In addition to promoting exhaustion of the TILs, high doses of IL2 can cause severe side effects in patients with cancer and often cannot be tolerated by those patients in need of ACT. The present compositions and methods provide a TIL therapy that optionally requires no exogenous cytokine administration, such as interleukins like IL2, before, during or after administration with the TILs. Stated differently, with the present method, there is no need for concomitant interleukin therapy with TIL infusion. For example, optionally the subject does not require administration of exogenous IL2 preceding TIL infusion, or for 5 days, 7 days, 10 days, 14 days, 21 days, or 28 days after TIL infusion. Similarly, the present method eliminates the need for infusion of modified IL2 or other modified cytokine (such as a modified IL7 or IL15). By way of example, a modified interleukin can be a mutant or fragment of IL2, IL7, or IL15 that retains one or more functions of IL2, IL7, or IL15 but has reduced binding affinity to certain receptors, such as receptors that can promote CD4+T_{reg} cell proliferation (e.g., by having reduced affinity).

As used herein, expansion refers to an increase in number or amount. When the term expansion is used herein in reference to a population or subpopulation of TILs, the term refers to a population of cells after REP. The size of the expanded population (i.e., the number of TILs after REP) is greater than an unexpanded population (i.e., the number of TILs pre-REP or the number of TILs after an unsuccessful REP resulting in the absence of a functional expansion of cells). When used in reference to a cell, such as an expanded TIL, it is a cell that has undergone and is the product or result of REP (i.e., culture with feeder cells and selected stimulatory factors) that has resulted in functional expansion of the TIL population. Thus, as used herein an expanded TIL is progeny of TILs (e.g., TILs that are modified to express mbIL15) cultured under REP resulting in functional expansion. Similarly, an unexpanded TIL as used herein refers to a TIL that has not undergone functional expansion in REP. Such an unexpanded TIL, however, may have gone through an initial IL2 pre-REP step or an unsuccessful REP resulting in the absence of a functional expansion of cells.

As used herein, the term expansion can be used quantitatively, such as expands more, expands less, greater expansion, less expansion, and the like. Such relative terms generally refer to a greater to lesser fold increase in the number of TILs in a population or subpopulation as compared to a different population or subpopulation (e.g., expansion of a modified TIL as compared to expansion of an unmodified TIL). Thus, for example, a greater expansion of a subpopulation of modified TILs as compared to unmodified TILs means a greater fold increase, such as 1.5-fold as compared to a 1.25-fold increase, a 2-fold increase as compared to a 1.5-fold increase, a 5-fold increase as compared to a 2-fold increase, a 10-fold increase as compared to a 5-fold increase, a 40-fold increase as compared to a 10-fold increase, and the like of the modified TILs as compared to the unmodified TILs.

The TILs described herein are engineered to express mbIL15. Thus, the TILs comprise an exogenous nucleic acid sequence that encodes IL15, an exogenous nucleic acid sequence that encodes a transmembrane domain, and, optionally, an exogenous nucleic acid sequence that encodes a linker, hinge, and/or leader sequence. IL15 is not generally expressed as a membrane bound molecule, thus, to express mbIL15, the IL15 must be associated with a transmembrane domain. IL15 as used herein refers to an IL15 polypeptide (e.g., UniProtKB-P40933 (IL15_HUMAN)). In one embodiment, the IL15 payload comprises the amino acid sequence provided in Table 2 (SEQ ID NO:12) or a polypeptide having at least 85, 90, 95, or 99% identity to SEQ ID NO: 12 that retains one or more IL15 functions (e.g., promoting expansion of modified TILs in vivo, promoting cytotoxicity of T and NK cells).

Exemplary transmembrane domains include a MHC1 transmembrane domain, a CD8 transmembrane domain, a B7-1 transmembrane domain, a CD4 transmembrane domain, a CD28 transmembrane domain, a CTLA-4 transmembrane domain, a PD-1 transmembrane domain, a human IgG4 Fc region, or an IL15 receptor subunit (e.g., IL15αR). The IL15 can be directly linked to the transmembrane domain or may be connected via a linker or hinge.

Numerous linker sequences (linkers) are known in the art. Linkers include, without limitation, GS linkers, GSG linkers, and GGSG linkers. These linkers are repeats of the subunit one or more times. Thus, a GS linker is a GSₙ linker where n is a numerical number being 1, 2, 3, 4, 5, 6, 7, 8, 9, 10 or more. Similarly, a GSG linker is a GSₙ linker wherein n is a numerical number being 1, 2, 3, 4, 5, 6, 7, 8, 9, 10, or more. A GGSG linker is a GGSGₙ linker where n is a numerical number being 1, 2, 3, 4, 5, 6, 7, 8, 9, 10 or more. The linker selection or linker length may influence activity level of the IL15 payload in the absence of ligand, and, the specific linker and length can be chosen to maximize the on state (e.g., maximum activity level) while maintaining low basal activity level and ligand (e.g., drug) responsiveness.

As yet another example, the specific hinge may allow for conformational changes and thereby influence ligand responsiveness and is thus chosen to result in a sufficient dynamic range to obtain a desired range of payload abundance and biologic activity (i.e., an acceptable payload activity range that corresponds to variation in ligand from zero or minimal to maximum saturation).

A hinge sequence is a short sequence of amino acids that facilitates flexibility between connected components. The hinge sequence can be any suitable sequence derived or obtained from any suitable molecule. The hinge sequence may be derived from all or part of an immunoglobulin (e.g., IgG1, IgG2, IgG3, IgG4) hinge region, i.e., the sequence that falls between the CHI and CH2 domains of an immunoglobulin (e.g., an IgG4 Fc hinge), or the extracellular regions of type 1 membrane proteins such as CD8α CD4, CD28 and CD7, which may be a wild type sequence or a derivative thereof. Some hinge regions include an immunoglobulin CH3 domain or both a CH3 domain and a CH2 domain. In some embodiments, the hinge is derived from a transmembrane domain.

The modified TILs described herein optionally further comprise an exogenous nucleic acid sequence that encodes an intracellular/cytoplasmic or transmembrane tail. Optionally, the intracellular/cytoplasmic or transmembrane tail is a B7.1, CD8, CD40L, LIGHT, or NKG2C intracellular tail.

The modified TILs described herein optionally further comprise an exogenous nucleic acid sequence that encodes a signal sequence (leader sequence). Exemplary leader sequences include MDMRVPAQLLGLLLLWLSGARC (SEQ ID NO:10), MDWTWILFLVAAATRVHS (IgEss: SEQ ID NO:58), MRISKPHLRSISIQCYLCLLLNSHFLTEAGIHVFILGCFSAGLPKTEA (Native IL15 LS; SEQ ID NO: 59), MGLVRRGARAGPRMPRGWTALCLLSLLPSGFMA (CD34: SEQ ID NO:60)

Additionally, certain TIL further comprise an exogenous nucleic acid sequence that encodes a DRD. IL15 is important for T cell and NK cell proliferation, but continuous exposure to high levels of IL15 may lead to exhaustion of these cells in vivo, which would decrease the efficacy of IL15 expressing TILs. Thus, in certain embodiments, a DRD is operably linked to the mbIL15 to provide regulation of the IL15 activity during TIL immunotherapy.

Drug responsive domains (DRDs) are polypeptides that regulate the expression or activity level of a payload. Although referred to as drug responsive domains, the ligand to which a DRD is responsive need not be an approved small molecule or biologic “drug.” More specifically, DRDs interact with a ligand such that, when the DRD is operatively linked to a payload, it confers ligand-dependent reversible regulation of a characteristic of the payload (for example, activity or abundance). U.S. Pat. Nos. 9,487,787 and 10,137,180, U.S. Publication Nos.: 2019/0192691; 2020/0101142; 2020/0172879; 2021/0069248, and U.S. Pat. No. 17,251,635; and Ser. No. 17/288,373, the contents of each of which are hereby incorporated by reference in their entirety, provide examples of DRDs (and their paired ligands) according to this disclosure. Certain of these and other example DRDs suitable for use according to this disclosure are also provided elsewhere in this specification. The DRDs, by way of example, can be chosen from FKBP (SEQ ID NO:4), ecDHFR (SEQ ID NO:1), hDHFR (SEQ ID NO:2), ER (SEQ ID NO: 9), PDE5 full-length (SEQ ID NO:6), PDE5 ligand binding domain (SEQ ID NO:5) and CA2 (SEQ ID NO:7) or a portion of any of the foregoing that maintains DRD function or an amino acid sequence having at least 85%, 86%, 87%, 88%, 89%, 90%, 91%, 92%, 93%, 94%, 95%, 96%, 97%, 98%, 99% or 100% identity to SEQ ID NOs: 1, 2, 4, 5, 6, 7, or 9 or the DRD functional portion thereof. The nucleic acid sequence for hDHFR is SEQ ID NO:3 and the nucleic acid sequence of CA2 is SEQ ID NO:8. One or more mutations (including truncations, substitutions, and deletions) in the amino acid sequence of FKBP, ecDHFR, hDHFR, ER, PDE5, and CA2, for example, can be advantageous to further destabilize the DRD. Suitable DRDs, which may be referred to as destabilizing domains or ligand binding domains, are also known in the art. See, e.g., WO2018/161000; WO2018/231759; WO2019/241315; U.S. Pat. No. 8,173,792: U.S. Pat. No. 8,530,636; WO2018/237323; WO2017/181119; US2017/0114346; US2019/0300864; WO2017/156238: Miyazaki et al., J Am Chem Soc, 134:3942 (2012); Banaszynski et al. (2006) Cell 126:995-1004; Stankunas, K. et al. (2003) Mol. Cell 12:1615-1624; Banaszynski et al. (2008) Nat. Med. 14:1123-1127; Iwamoto et al. (2010) Chem. Biol. 17:981-988; Armstrong et al. (2007) Nat. Methods 4:1007-1009; Madeira da Silva et al. (2009) Proc. Natl. Acad. Sci. USA 106:7583-7588; Pruett-Miller et al. (2009) PLOS Genet. 5: e1000376; and Feng et al. (2015) Elife 4: e10606, the contents of each of which are hereby incorporated by reference in their entirety.

Without meaning to be limited by theory, DRDs are thought to be unstable polypeptides that degrade in the absence of their corresponding stabilizing ligand (also referred to as the paired ligand or ligand), but whose stability is rescued by binding to the stabilizing ligand. Because binding of the ligand to the DRD is reversible, later removal of the ligand results in the DRD unfolding, becoming unstable, and ultimately being tagged for degradation by the ubiquitin-proteasome system (“UPS”). Accordingly, it is believed that when a DRD is operably linked to a payload like mbIL15, the entire construct (i.e., DRD plus IL15) itself is rendered unstable and degraded by the UPS. However, in the presence of the paired ligand, the construct is stabilized and the mbIL15 payload remains available. Further, it is believed that the conditional nature of DRD stability allows a rapid and non-perturbing switch from stable protein to unstable UPS substrate and may facilitate regulation or modulation of a payload's activity level, and/or modulation of a payload's activity level.

Because the abundance and availability of a payload are related to the activity of a payload, for purposes of this disclosure, the terms abundance, availability, activity, and the phrase abundance and/or activity (and similarly level of abundance, level of availability, level of activity, and level of abundance and/or activity) are used interchangeably throughout this disclosure and are generally referred to as activity, unless explicitly stated otherwise or nonsensical in context. Further, measurements of abundance or availability are used as a proxy for activity level and may be used herein to reflect the activity level. Consequently, changes in the abundance or availability of a payload in the presence of an effective amount of ligand as compared to in the absence of ligand optionally serves as a proxy for measuring changes in activity level.

Numerous DRDs are described herein, but one of skill in the art could identify additional DRDs. By way of example, DRDs can be identified using library screening and structure-guided engineering to select the optimal DRD variant with sufficient instability in the absence of the ligand and sufficient stability in the presence of the ligand. A variant library can be generated using random mutagenesis screening by transducing cells (e.g., Jurkat cells) with mutant DRD candidates. To produce an enriched library, cells with the desired characteristics (low basal activity/expression and high dynamic range) are then selected by testing expression of a reporter gene across a range of concentrations of ligand. Single cell clones are then produced and characterized to identify candidate DRDs. The DRD is capable of affecting a characteristic, for example, the abundance or activity level, of a payload to which it is operably linked. Further, the one or more DRDs interact with a ligand to provide ligand-dependent reversible regulation of the characteristic of the payload. The DRDs described herein are responsive to a paired ligand. Optionally, the DRDs are responsive to a paired ligand that is a small molecule drug, such as an FDA-approved small molecule. However, one of skill in the art can select the DRD and its paired ligand to meet the specific needs of the system. Examples of stabilizing ligands and their uses for specific DRDs described herein are shown in Table 1 and in U.S. Pat. No. 9,487,787 filed Mar. 33, 2012, U.S. Pat. No. 10,137,180 filed Sep. 6, 2013, PCT Application No. PCT/US2018/037005, filed Jun. 12, 2018, PCT Application No. PCT/US2019/036654 filed Jun. 12, 2019, PCT Application No. PCT/US2019/057698 filed Oct. 23, 2019, PCT Application No. PCT/US2020/021596 filed Mar. 6, 2020, and U.S. application Ser. No. 16/558,224 filed Sep. 2, 2019, the disclosures of all of the aforereferenced applications are incorporated herein by reference in their entireties.

TABLE 1
Listing of DRD and exemplary ligands

| | | Exemplary |
|---|---|---|
| DRD Protein | SEQ ID NO: | Ligands |
| E. coli dihydrofolate reductase | 1 | Methotrexate |
| (ecDHFR) (Uniprot ID: P0ABQ4) | | (MTX) |
| | | Trimethoprim |
| | | (TMP) |
| Human dihydrofolate reductase | 2 | Methotrexate |
| (hDHFR) (Uniprot ID: P00374) | | (MTX) |
| | | Trimethoprim |
| | | (TMP) |
| Human FKBP (FK506 binding protein) | 4 | Shield-1 |
| (Uniprot ID: P62942) | | |
| Phosphodiesterase 5 (PDE5), | 5 | Sildenafil; |
| ligand binding domain | | Vardenafil; |
| (Uniprot ID: Uniprot ID O76074) | | Tadalafil |
| Phosphodiesterase 5 (PDE5), full-length | 6 | Sildenafil; |
| (Uniprot ID: Uniprot ID O76074) | | Vardenafil; |
| | | Tadalafil |
| Carbonic anhydrase II (CA2) | 7 | Celecoxib |
| (Uniprot ID: P00918) | | Acetazolamide |
| Human estrogen receptor (ER) | 9 | Bazedoxifene |
| Uniprot ID: P03372.2) | | Raloxifene |

Optionally, a DRD of the present disclosure may be derived from human carbonic anhydrase 2 (hCA2), which is a member of the carbonic anhydrases, a superfamily of metalloenzymes. A DRD of the present disclosure may be derived from amino acids 1-260 of CA2 (Uniprot ID: P00918) (SEQ ID NO:7). Optionally, DRDs are derived from CA2 comprising amino acids 2-260 of the parent CA2 sequence (e.g., amino acids 2-260). This is referred to herein as a CA2 M1del mutation (CA2; SEQ ID NO:55). Optionally, a DRD of the present disclosure comprises a region of or the whole human carbonic anhydrase 2, and further comprises one or more mutations relative to the full-length sequence selected from M1del, L156H, and S56N. Optionally, the DRD is selected from the group consisting of SEQ ID NOs: 7, 26 (CA2 M1del and L156H), 55, 56 (CA2 S56N), and 57 (CA2 M1del and S56N).

By way of example, the modified TIL can comprise a nucleic acid sequence that encodes a transmembrane domain that is C-terminal to the IL15 polypeptide component and an intracellular tail that is C-terminal to the transmembrane domain.

Non-limiting examples of constructs and construct components for the modified TILs are shown in Table 2. The construct designated OT-IL15-292 includes from the N terminus a signal sequence, IL15, (GS) 15 linker, a hinge region, a transmembrane region, and an intracellular tail. The construct designated OT-IL15-293 includes a DRD (specifically, a CA2 DRD (M1del, L156H)) at the C terminus.

TABLE 2
Examples of constructs and construct components

| | | | Amino | Nucleic |
|---|---|---|---|---|
| | | | Acid | Acid |
| | Amino Acid | | SEQ ID | SEQ ID |
| Description | Sequence (AA) | Nucleic Acid Sequence (NA) | NO | NO |
| Leader | MDMRVPAQLLGL | ATGGACATGCGGGTGCCTGCACAACTTC | 10 | 11 |
| sequence | LLLWLSGARC | TGGGCCTGCTGTTGTTGTGGCTGTCTGGA | | |
| | | GCCCGGTGT | | |
| Interleukin-15 | NWVNVISDLKKIE | AATTGGGTAAATGTTATCAGTGATCTCAA | 12 | 13 |
| (IL15) | DLIQSMHIDATLY | GAAGATAGAGGATCTCATCCAGTCCATG | | |
| | TESDVHPSCKVTA | CATATTGATGCCACGCTGTACACAGAAA | | |
| | MKCFLLELQVISLE | GCGATGTGCATCCTAGCTGTAAGGTGAC | | |
| | SGDASIHDTVENLI | AGCGATGAAGTGTTTTCTTTTGGAGCTGC | | |
| | ILANNSLSSNGNV | AGGTAATTAGTCTTGAGTCCGGCGATGC | | |
| | TESGCKECEELEE | CAGCATTCATGATACCGTAGAAAACTTG | | |
| | KNIKEFLQSFVHIV | ATTATCCTGGCCAACAATTCTCTGTCCTC | | |
| | QMFINTS | AAACGGAAACGTAACCGAGAGCGGTTGT | | |
| | | AAAGAATGTGAAGAACTGGAAGAAAAG | | |
| | | AACATCAAGGAGTTTCTGCAATCATTCGT | | |
| | | TCACATCGTACAAATGTTCATAAATACGT | | |
| | | CA | | |
| Linker (GS)15 | GSGSGSGSGSGSG | GGATCTGGTTCTGGTTCCGGAAGTGGATC | 14 | 15 |
| | SGSGSGSGSGSGS | TGGTTCAGGGTCCGGTAGTGGATCTGGG | | |
| | GSGS | TCAGGAAGTGGAAGCGGTAGTGGGTCTG | | |
| | | GATCT | | |
| Hinge | KQEHFPDN | AAACAAGAGCACTTTCCTGATAAC | 16 | 17 |
| Transmembrane | LLPSWAITLISVNG | CTGTTGCCGAGCTGGGCGATTACGCTTAT | 18 | 19 |
| | IFVICCL | CAGTGTAAACGGCATCTTTGTAATATGCT | | |
| | | GTCTG | | |
| Intracellular | TYCFAPRCRERRR | ACCTACTGCTTCGCACCAAGGTGCCGGG | 20 | 21 |
| tail | NERLRRESVRPV | AGAGAAGGAGAAATGAAAGACTGAGAA | | |
| | | GGGAGAGCGTGAGACCTGTG | | |
| Intracellular | TYCFAPRCRERAR | ACCTACTGCTTCGCACCAAGGTGCCGGG | 22 | 23 |
| tail | NERLRRETVRPV | AGAGAGCAAGAAATGAAAGACTGAGAA | | |
| | | GGGAGACCGTGAGACCTGTG | | |
| Linker (GS) | GS | GGATCC | 24 | 25 |
| Drug | SHHWGYGKHNGP | TCCCATCACTGGGGGTACGGCAAACACA | 26 | 27 |
| Responsive | EHWHKDFPIAKGE | ACGGACCTGAGCACTGGCATAAGGACTT | | |
| Domain (CA2 | RQSPVDIDTHTAK | CCCCATTGCCAAGGGAGAGCGCCAGTCC | | |
| (M1del, | YDPSLKPLSVSYD | CCTGTTGACATCGACACTCATACAGCCA | | |
| L156H)) | QATSLRILNNGHA | AGTATGACCCTTCCCTGAAGCCCCTGTCT | | |
| | FNVEFDDSQDKAV | GTTTCCTATGATCAAGCAACTTCCCTGAG | | |
| | LKGGPLDGTYRLI | AATCCTCAACAATGGTCATGCTTTCAACG | | |
| | QFHFHWGSLDGQ | TGGAGTTTGATGACTCTCAGGACAAAGC | | |
| | GSEHTVDKKDSIK | AGTGCTCAAGGGAGGACCCCTGGATGGC | | |
| | TKGKSADFTNFDP | ACTTACAGATTGATTCAGTTTCACTTTCA | | |
| | RGLLPESLDYWTY | CTGGGGTTCACTTGATGGACAAGGTTCA | | |
| | PGSLTTPPLLECVT | GAGCATACTGTGGATAAAAAGAAATATG | | |
| | WIVLKEPISVSSEQ | CTGCAGAACTTCACTTGGTTCACTGGAAC | | |
| | VLKFRKLNFNGEG | ACCAAATATGGGGATTTTGGGAAAGCTG | | |
| | EPEELMVDNWRP | TGCAGCAACCTGATGGACTGGCCGTTCT | | |
| | AQPLKNRQIKASF | AGGTATTTTTTTGAAGGTTGGCAGCGCTA | | |
| | K | AACCGGGCCATCAGAAAGTTGTTGATGT | | |
| | | GCTGGATTCCATTAAAACAAAGGGCAAG | | |
| | | AGTGCTGACTTCACTAACTTCGATCCTCG | | |
| | | TGGCCTCCTTCCTGAATCCCTGGATTACT | | |
| | | GGACCTACCCAGGCTCACTGACCACCCC | | |
| | | TCCTCTTCTGGAATGTGTGACCTGGATTG | | |
| | | TGCTCAAGGAACCCATCAGCGTCAGCAG | | |
| | | CGAGCAGGTGTTGAAATTCCGTAAACTT | | |
| | | AACTTCAATGGGGAGGGTGAACCCGAAG | | |
| | | AACTGATGGTGGACAACTGGCGCCCAGC | | |
| | | TCAGCCACTGAAGAACAGGCAAATCAAA | | |
| | | GCTTCCTTCAAA | | |
| OT-IL15-292 | MDMRVPAQLLGL | ATGGACATGCGGGTGCCTGCACAACTTC | 28 | 29 |
| | LLLWLSGARCNW | TGGGCCTGCTGTTGTTGTGGCTGTCTGGA | | |
| | VNVISDLKKIEDLI | GCCCGGTGTAATTGGGTAAATGTTATCA | | |
| | QSMHIDATLYTES | GTGATCTCAAGAAGATAGAGGATCTCAT | | |
| | DVHPSCKVTAMK | CCAGTCCATGCATATTGATGCCACGCTGT | | |
| | CFLLELQVISLESG | ACACAGAAAGCGATGTGCATCCTAGCTG | | |
| | DASIHDTVENLIIL | TAAGGTGACAGCGATGAAGTGTTTTCTTT | | |
| | ANNSLSSNGNVTE | TGGAGCTGCAGGTAATTAGTCTTGAGTCC | | |
| | SGCKECEELEEKNI | GGCGATGCCAGCATTCATGATACCGTAG | | |
| | KEFLQSFVHIVQM | AAAACTTGATTATCCTGGCCAACAATTCT | | |
| | FINTSGSGSGSGSG | CTGTCCTCAAACGGAAACGTAACCGAGA | | |
| | SGSGSGSGSGSGS | GCGGTTGTAAAGAATGTGAAGAACTGGA | | |
| | GSGSGSGSKQEHF | AGAAAAGAACATCAAGGAGTTTCTGCAA | | |
| | PDNLLPSWAITLIS | TCATTCGTTCACATCGTACAAATGTTCAT | | |
| | VNGIFVICCLTYCF | AAATACGTCAGGATCTGGTTCTGGTTCCG | | |
| | APRCRERRRNERL | GAAGTGGATCTGGTTCAGGGTCCGGTAG | | |
| | RRESVRPVGS | TGGATCTGGGTCAGGAAGTGGAAGCGGT | | |
| | | AGTGGGTCTGGATCTAAACAAGAGCACT | | |
| | | TTCCTGATAACCTGTTGCCGAGCTGGGCG | | |
| | | ATTACGCTTATCAGTGTAAACGGCATCTT | | |
| | | TGTAATATGCTGTCTGACCTACTGCTTCG | | |
| | | CACCAAGGTGCCGGGAGAGAAGGAGAA | | |
| | | ATGAAAGACTGAGAAGGGAGAGCGTGA | | |
| | | GACCTGTGGGATCC | | |
| OT-IL15-293 | MDMRVPAQLLGL | ATGGACATGCGGGTGCCTGCACAACTTC | 30 | 31 |
| | LLLWLSGARCNW | TGGGCCTGCTGTTGTTGTGGCTGTCTGGA | | |
| | VNVISDLKKIEDLI | GCCCGGTGTAATTGGGTAAATGTTATCA | | |
| | QSMHIDATLYTES | GTGATCTCAAGAAGATAGAGGATCTCAT | | |
| | DVHPSCKVTAMK | CCAGTCCATGCATATTGATGCCACGCTGT | | |
| | CFLLELQVISLESG | ACACAGAAAGCGATGTGCATCCTAGCTG | | |
| | DASIHDTVENLIIL | TAAGGTGACAGCGATGAAGTGTTTTCTTT | | |
| | ANNSLSSNGNVTE | TGGAGCTGCAGGTAATTAGTCTTGAGTCC | | |
| | SGCKECEELEEKNI | GGCGATGCCAGCATTCATGATACCGTAG | | |
| | KEFLQSFVHIVQM | AAAACTTGATTATCCTGGCCAACAATTCT | | |
| | FINTSGSGSGSGSG | CTGTCCTCAAACGGAAACGTAACCGAGA | | |
| | SGSGSGSGSGSGS | GCGGTTGTAAAGAATGTGAAGAACTGGA | | |
| | GSGSGSGSKQEHF | AGAAAAGAACATCAAGGAGTTTCTGCAA | | |
| | PDNLLPSWAITLIS | TCATTCGTTCACATCGTACAAATGTTCAT | | |
| | VNGIFVICCLTYCF | AAATACGTCAGGATCTGGTTCTGGTTCCG | | |
| | APRCRERRRNERL | GAAGTGGATCTGGTTCAGGGTCCGGTAG | | |
| | RRESVRPVGSSHH | TGGATCTGGGTCAGGAAGTGGAAGCGGT | | |
| | WGYGKHNGPEHW | AGTGGGTCTGGATCTAAACAAGAGCACT | | |
| | HKDFPIAKGERQS | TTCCTGATAACCTGTTGCCGAGCTGGGCG | | |
| | PVDIDTHTAKYDP | ATTACGCTTATCAGTGTAAACGGCATCTT | | |
| | SLKPLSVSYDQAT | TGTAATATGCTGTCTGACCTACTGCTTCG | | |
| | SLRILNNGHAFNV | CACCAAGGTGCCGGGAGAGAAGGAGAA | | |
| | EFDDSQDKAVLKG | ATGAAAGACTGAGAAGGGAGAGCGTGA | | |
| | GPLDGTYRLIQFHF | GACCTGTGGGATCCTCCCATCACTGGGG | | |
| | HWGSLDGQGSEH | GTACGGCAAACACAACGGACCTGAGCAC | | |
| | TVDKKDSIKTKGK | TGGCATAAGGACTTCCCCATTGCCAAGG | | |
| | SADFTNFDPRGLL | GAGAGCGCCAGTCCCCTGTTGACATCGA | | |
| | PESLDYWTYPGSL | CACTCATACAGCCAAGTATGACCCTTCCC | | |
| | TTPPLLECVTWIVL | TGAAGCCCCTGTCTGTTTCCTATGATCAA | | |
| | KEPISVSSEQVLKF | GCAACTTCCCTGAGAATCCTCAACAATG | | |
| | RKLNFNGEGEPEE | GTCATGCTTTCAACGTGGAGTTTGATGAC | | |
| | LMVDNWRPAQPL | TCTCAGGACAAAGCAGTGCTCAAGGGAG | | |
| | KNRQIKASFK | GACCCCTGGATGGCACTTACAGATTGATT | | |
| | | CAGTTTCACTTTCACTGGGGTTCACTTGA | | |
| | | TGGACAAGGTTCAGAGCATACTGTGGAT | | |
| | | AAAAAGAAATATGCTGCAGAACTTCACT | | |
| | | TGGTTCACTGGAACACCAAATATGGGGA | | |
| | | TTTTGGGAAAGCTGTGCAGCAACCTGAT | | |
| | | GGACTGGCCGTTCTAGGTATTTTTTTGAA | | |
| | | GGTTGGCAGCGCTAAACCGGGCCATCAG | | |
| | | AAAGTTGTTGATGTGCTGGATTCCATTAA | | |
| | | AACAAAGGGCAAGAGTGCTGACTTCACT | | |
| | | AACTTCGATCCTCGTGGCCTCCTTCCTGA | | |
| | | ATCCCTGGATTACTGGACCTACCCAGGCT | | |
| | | CACTGACCACCCCTCCTCTTCTGGAATGT | | |
| | | GTGACCTGGATTGTGCTCAAGGAACCCA | | |
| | | TCAGCGTCAGCAGCGAGCAGGTGTTGAA | | |
| | | ATTCCGTAAACTTAACTTCAATGGGGAG | | |
| | | GGTGAACCCGAAGAACTGATGGTGGACA | | |
| | | ACTGGCGCCCAGCTCAGCCACTGAAGAA | | |
| | | CAGGCAAATCAAAGCTTCCTTCAAA | | |

To create a membrane-tethered cytokine like IL15 operably linked to a DRD with a sufficient dynamic range (i.e., an acceptable activity range that corresponds to variation in ligand from zero to maximum saturation), the polypeptide optionally includes from the N-terminus the payload (IL15), a linker, a hinge, a transmembrane region, a tail, and a DRD. The tail and/or linker and tail and linker length may influence the activity level in the absence of ligand and in some embodiments, the specific tail and/or linker and length are chosen to maximize the on-state (e.g., maximum activity level) while maintaining low basal activity level and ligand responsiveness. The specific hinge may allow for conformational changes and thereby influence ligand responsiveness across a sufficient dynamic range.

The modified TILs that express mbIL15 as described herein have a number of advantages. First, the modified TILs can be expanded in vitro in the presence of feeder cells (such as K562 feeder cells that express 41BBL and IL21 (optionally, mbIL21)). Significantly, subsequent to the pre-REP stage, the modified TILs can expand in vitro in the absence of exogenous cytokine and the expanded TILs are activated and can expand further in vivo without administration of an exogenous cytokine, such as IL2. The result is that the TILs are less exhausted and can more readily expand and infiltrate the target tumor.

A population of TILs comprising a plurality of modified TILs can include a subpopulation of the TILs that has undergone expansion (i.e., REP with feeder cells and stimulatory molecules, such as IL21 and 41BBL). The expanded TILs demonstrate a number of advantages. For example, expanded TILs that have undergone REP are then capable of surviving in a culture lacking feeder cells. More particularly, TILs engineered to express mbIL15 can survive longer than unengineered cells in the absence of an exogenous cytokine, for example, an interleukin such as IL2. TILs engineered to express mbIL15 operably linked to a DRD survive better in the presence of the ligand but in the absence of exogenous cytokine than unengineered TILs. Additionally, a population of expanded TILs shows preferential expansion of certain TILs and, thus, fewer or more subtypes of TILs as compared to a control population of unexpanded TILs. A control population of unexpanded TILs as used herein refers to TILs that are similarly modified as the expanded TILs but that have not undergone REP.

A population of expanded TILs, for example, has a greater proportion of CD8+ cells, a lesser proportion of CD4+ cells, and a lower CD4+:CD8+ratio as compared to a control population of unexpanded TILs. CD8+TILs are considered key players in killing cancer cells by releasing cytotoxic molecules and cytokines, and the number of CD4+TILs compared to the number of CD8+TILs (i.e., the CD4+:CD8+ratio) in a tumor has been found to correlate with a positive outcome.

Additionally, in certain embodiments, the population of expanded TILs has a lesser proportion of T_{reg} cells as compared to the proportion of T_{reg} cells in a control population of unexpanded TILs. T_{reg} cells have a role in immunological tolerance and immune homeostasis by suppressing immune reactions. Thus, a lower proportion of T_{reg} cells is desirable in immunotherapy such as ACT with TILs.

The population of expanded TILs also shows fewer exhausted TILs and more polyfunctional TILs. The population of expanded TILs a lesser proportion of PD1+ cells as compared to the proportion of PD1+ cells in a control population of unexpanded TILs. Additionally, the population of expanded TILs has a greater proportion of cells positive for tumor necrosis factor α (TNFα) and interferon γ (IFNγ) as compared to the proportion of TILs positive for TNFα and IFNγ in a control population of unexpanded TILs.

Provided herein is a mixed population of TILs comprising a subpopulation of unmodified TILs and a subpopulation of modified TILs comprising a mbIL15, which is optionally operably linked to a DRD. The subpopulation of modified TILs expands in the presence of K562 feeder cells expressing 41BBL and IL21. The subpopulation of modified TILs expands more than the subpopulation of unengineered TILs in the presence of K562 feeder cells expressing 41BBL, and IL21. This preferential expansion of modified TILs occurs in the absence of exogenous IL2 during the REP.

### Methods of Making Modified Tumor Infiltrating Lymphocytes (TILs)

Allogeneic TILs can be derived from a donor tumor or a biopsy thereof using methods known in the art. For example, pieces of the tumor (e.g., 1-5 mm in size) are subjected to enzymatic digestion (e.g., collagenase (0.5-5 mg/mL), DNAse, hyaluronidase) or mechanical dissociation. The dissociated cells are incubated in cell culture media under conditions that favor the proliferation of TILs over other cells (i.e., in the presence of IL2). This stage is the pre-REP stage. In the pre-REP stage, the cells can be cultured (e.g., 3 to 28 days) in the presence of 2000-8000 IU/mL IL2 (e.g., 6000 IU/ml) and optionally in the presence of inactivated human AB serum. In some embodiments, the cells are cultured for a period of days, generally from 3 to 28 days. In some embodiments, this pre-REP cell population is cultured for a period of 7 to 21 days.

The pre-REP TILs can be cryopreserved. Cryopreserved cells are thawed and rested before activation. The cells can be activated using, for example, plate bound OKT3, soluble OKT3, costimulatory antibodies (e.g., antibodies to CD28 or 41BB)+OKT3, anti-CD3 and anti-CD28 antibodies bound to bead or fragments, etc. The activation step can be 1-2 days or longer. Following activation, one or more TILs are then engineered to express a membrane-bound interleukin 15 (mbIL15) by transducing the one or more TILs with a vector having a first nucleic acid sequence that encodes IL15 and a second nucleic acid sequence that encodes a transmembrane domain. Optionally the vector further comprises one or more nucleic acid sequences that encode a signal peptide, a linker, a hinge, an intracellular tail, or a DRD. The vector can be configured any number of ways to achieve the desired mbIL15. Exemplary nucleic acid constructs include the nucleic acid sequences encoding OT-IL15-293 and OT-IL15-292, with and without DRDs, respectively. Thus, the vector optionally comprises SEQ ID NO:29, 31, 53 or 54. The vector includes or encode additional elements, such as a promoter sequence and other regulatory elements (enhancers, translational control elements (e.g., IRES), and elements that control half-life.)). The vector optionally comprises or can comprises nucleic acid sequences that encode elements that control translation (e.g., IRES, WPRE, and the like).

The vector can be chosen from viral vectors, plasmids, cosmids, and artificial chromosomes. By way of example, the vector can be a viral vector, such as a lentiviral vector or a retroviral vector. By way of example the viral vector can a baboon endogenous retrovirus envelope (BaEV) pseudotyped lentiviral vector that comprises a nucleic acid that encodes IL15 and a nucleic acid sequence that encodes a transmembrane domain. Upon expression, the IL15 is associated with the transmembrane domain and is membrane bound by the transmembrane domain.

Vectors are optionally transferred to cells by non-viral methods by physical methods such as needles, electroporation, sonoporation, hydroporation, chemical carriers (such as inorganic particles (e.g., calcium phosphate, silica, gold)), and/or chemical methods. In some embodiments, synthetic or natural biodegradable agents are used for delivery such as cationic lipids, lipid nano emulsions, nanoparticles, peptide-based vectors, or polymer-based vectors.

The nucleic acid sequence that encodes IL15 can be genomic or non-genomic. That is, the delivery system used to deliver the IL15 encoding nucleic acid sequence can be integrated into the genome of the TIL or can be non-integrated (i.e., episomal).

The TIL comprising mbIL15 are expanded in the REP stage (e.g., 5-21 days or any amount in between, including 7-14 days). As described further in the Examples, the TILs modified to express IL15 are expanded in the presence of K562 feeder cells as well as 41BBL and IL21. In certain embodiments, the K562 feeder cells are engineered to express 41BBL and IL21, which can be membrane bound IL21 (mbIL21), thus reducing or eliminating the need for exogenous cytokines such as IL2, IL7, or IL15 during the REP. In some embodiments, the modified TILs are cultured with the K562 cells modified to express mbIL21 and 41BBL at a ratio of 1:1 to 100:1, 1:1 to 50:1, 1:1 to 20:1, 1:1 to 10:1, or 2:1 to 5:1 (TIL:feeder cell).

Before feeder cells are used in the present method, they are first rendered replication incompetent. Various means of treating the feeder cells are known in the art. Such methods include irradiation (e.g., with gamma or X-rays), mitomycin-C treatment, electric pulses, mild chemical fixation (e.g., with formaldehyde or glutaraldehyde), or transduction of the feeder cells with a suicide gene. In some embodiments, the feeder cells are human cells. By way of example, the irradiation can be at 25-300 Gy delivered for example by a cesium source or an X-ray source.

Following expansion on feeder cells, the TILs are optionally derived from the feeder cells. As used herein, the term isolation is not meant to suggest that the TILs are entirely free of other components, such as feeder cells, just to suggest that the TILs are relatively free of feeder cells.

Provided herein is a method of making a TIL, a population of TILs, or a subpopulation of TILs that comprise mbIL15. Also provided are nucleic acid sequences encoding the mbIL15, vectors comprising the nucleic acid sequence encoding the mbIL15, replication incompetent K562 feeder cells that are modified to express 41BBL and IL21, and TILs made by the method described herein.

### Pharmaceutical Compositions

Provided herein is a pharmaceutical composition suitable for use in ACT. The pharmaceutical composition can comprise allogeneic TILs, such as expanded TILs, and a pharmaceutically acceptable carrier. The population of allogeneic TILs in the pharmaceutical composition is optionally a mixed population of TILs comprising a subpopulation of modified TILs (i.e., TILs engineered to express IL15) and unmodified TILs (i.e., untransduced or unengineered TILs).

The term carrier means a compound, composition, substance, or structure that, when in combination with a compound or cells, aids or facilitates preparation, storage, administration, delivery, effectiveness, selectivity, or any other feature of the compound or cells for its intended use or purpose. For example, a carrier can be selected to minimize any degradation of the TILs and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water. By pharmaceutically acceptable is meant a material that is not biologically or otherwise undesirable, which can be administered to an individual along with TIL or TIL population without causing unacceptable biological effects or interacting in a deleterious manner with the TILs.

Optionally the pharmaceutical composition further comprises a cryoprotectant (cryopreservant). Such a cryoprotectant serves to prevent unacceptable cell lysis or damage should the TILs be frozen for future use. Cryoprotectants are known in the art. Such cryoprotectants can be selected from among glycerol, ethylene glycol, propylene glycol, or dimethylsulfoxide (DMSO).

The pharmaceutical compositions described herein optionally further comprise one or more pharmaceutically acceptable excipients ((e.g., human serum albumin or polymeric materials (e.g., PEG)).

The compositions of the present disclosure can be formulated in any manner suitable for delivery. The TILs can be administered in nanoparticles, poly (lactic-co-glycolic acid) (PLGA) microspheres, lipidoids, lipoplex, liposome, polymers, carbohydrates (including simple sugars), cationic lipids, or combinations thereof.

Although the descriptions of pharmaceutical compositions provided herein are principally directed to pharmaceutical compositions suitable for administration to humans, it will be understood by the skilled artisan that such compositions are generally suitable for administration to any other animal, e.g., to non-human mammals. Subjects to which administration of the pharmaceutical compositions is contemplated include, but are not limited to, agricultural animals, such as cattle, horses, chickens and pigs; domestic animals, such as cats, dogs; or research animals such as mice, rats, rabbits, dogs and non-human primates.

### Methods of Treatment

Provided herein are methods of treating cancer in a recipient subject by administering to the subject (i.e., the recipient subject) a modified allogeneic TIL that expresses mbIL15 or a population thereof, including a population or subpopulation of expanded TILs, or a pharmaceutical composition thereof. The cancer can be, but is not limited to, melanoma, uveal (ocular) melanoma, cervical cancer, ovarian cancer, head and neck cancer, lung cancer, non-small cell lung cancer (NSCLC), bladder cancer, breast cancer, renal cell carcinoma, pancreatic cancer, prostate cancer, cancer of the central nervous system, gastrointestinal cancer (e.g., colorectal cancer).

TIL therapy to date has required concomitant administration of high doses of IL2 simultaneously with and subsequent to administration of the TILs. But unlike conventional treatment with TILs, the present method does not require administration of IL2. Rather, the modified TILs by expressing mbIL15, provide a sufficient source of cytokine to stimulate proliferation and activity of the TILs.

The method of treating cancer can further comprise isolating one or more TILs from a donor tumor as described herein and introducing into the one or more TILs a nucleic acid sequence that encodes mbIL15. The TILs can be derived from a tumor of a donor subject (an allogeneic source). The tumor from which the TILs are derived can be a primary tumor or a metastatic tumor. TILs derived from the same tumor to be treated have the advantage of having neoantigens and heterogeneity that are the same as the tumor; however, TILs derived from a different tumor of the same subject can be selected for common cancer antigens as described herein. TILs from a tumor of a donor subject can be selected for HLA matching and/or efficacy against cancer antigens present in the tumor of the recipient subject. By common cancer antigens as used herein is meant that the TILs comprise t-cell receptors (TCR) that are specific for the cancer antigens in the tumor to be treated. Similarly, common cancer antigens can be present in a cell line and in the tumor of the recipient subject.

TILs can be obtained from a tumor sample surgical resection, tissue biopsy, needle biopsy or other means as an initial step. The TILs are then transduced as described herein and then expanded in vitro to provide a larger population of cells for ACT. Thus, TILs from one donor can be used for treating multiple recipients.

Administration of the modified TILs can include an amount from about 1000 cells/injection to up to about 10 billion cells/injection, such as 2×10¹¹, 1×10¹¹, 1×10¹⁰, 1×10⁹, 1×10⁸, 1×10⁷, 5×10⁷, 1×10⁶, 5×10⁶, 1×10⁵, 5×10⁵, 1×10⁴, 5×10⁴, 1×10³, 5×10³, cells per injection, or any ranges between any two of the numbers, end points inclusive. Optionally, from about 1×10⁸ to about 1×10¹¹ cells are administered to the subject.

TILs of the present disclosure can be administered by any suitable route. In some embodiments, the TILs are administered by intravenous infusion, intra-arterial infusion, intraperitoneally, intrathecally, intralymphatically. In some embodiments, the TILs are administered by intravenous or intra-arterial infusion. Optionally the TILs are administered locally, for example, directly into a tumor or blood vessel that supplies a tumor.

The TILs can be administered in a single dose, but in certain instances may be administered in multiple doses.

The method of treatment can further comprise lymphodepletion of the recipient subject prior to administration of the TILs. Investigations in humans and murine models of melanoma suggest that lymphodepletion depletes negative regulatory cells including regulatory T cells (T_{reg} cells) and peripheral myeloid-derived suppressor cells, which can suppress T cell proliferation. Thus, lymphodepletion aids in the proliferation of adoptively transferred TILs. Lymphodepleting conditioning regimen include, for example, pre-treatment of the recipient subject with full body irradiation or lymphodepleting agents before adoptive transfer of the TILs. This preconditioning allows the TILs to expand by eliminating T_{reg} cells and removing potential cytokine sinks by which normal cells compete with the newly infused TILs.

One example of a lymphodepleting agent is fludarabine (e.g., at a dose of 0.5 μg/ml-10 μg/ml). In some embodiments, the fludarabine is administered at a concentration of 1 μg/ml daily for 1-7 days before TIL:administration. In some embodiments, the fludarabine is administered at a dosage of 10 mg/kg/day, 15 mg/kg/day, 20 mg/kg/day, 25 mg/kg/day, 30 mg/kg/day, 35 mg/kg/day, 40 mg/kg/day, or 45 mg/kg/day. In some embodiments, the fludarabine treatment is administered for 2-7 days at 35 mg/kg/day. In some embodiments, the fludarabine treatment is administered for 4-5 days at 35 mg/kg/day. In some embodiments, the fludarabine treatment is administered for 4-5 days at 25 mg/kg/day.

In some embodiments, cyclophosphamide is administered to provide mafosfamide, its active form, at a concentration of 0.5 μg/mL-10 μg/mL. In some embodiments, cyclophosphamide is administered to provide mafosfamide at a concentration of 1 μg/mL daily for 1-7 days before TIL administration. In some embodiments, the cyclophosphamide is administered at a dosage of 50 mg/m²/day, 75 mg/m²/day, 100 mg/m²/day, 150 mg/m²/day, 175 mg/m²/day, 200 mg/m²/day, 225 mg/m²/day, 250 mg/m²/day, 275 mg/m²/day, or 300 mg/m²/day. In some embodiments, the cyclophosphamide is administered intravenously (i.v.). In some embodiments, the cyclophosphamide treatment is administered for 2-7 days at 35 mg/kg/day i.v. In some embodiments, the cyclophosphamide treatment is administered for 4-5 days at 250 mg/m²/day i.v. In some embodiments, the cyclophosphamide treatment is administered for 4 days at 250 mg/m²/day i.v.

In certain embodiments, lymphodepletion comprising administration of a combination of lymphodepleting agents, such as cyclophosphamide at 60 mg/kg for 2 days and fludarabine at 25 mg/m² for 5 days or cyclophosphamide 250 mg/m²/day for 4 days and fludarabine at 25 mg/m² for 4 days.

If the IL15 expressed by the TIL is operably linked to a DRD, the method can further comprise administering to the recipient subject a second agent (ligand) that binds to the DRD in an amount effective to increase the IL15 activity of the TIL. The ligand can be administered using a dosing regimen that provides a selected amount IL15 activity to the subject. The ligand can be delivered to achieve continuous or intermittent IL15 activity in the subject. Determining the frequency and duration of dosing to the subject is determined by a person of skill in the art by considering, for example, providing a higher dose or longer duration of administration of the ligand when more activity of the IL15 is desired and reduces or eliminates the ligand administration when less activity is desired. The dose and duration of ligand administration and the resulting activity of the IL15 is also selected to avoid unacceptable side effects or toxicity in the subject. Thus, the subject is administered an effective amount of the ligand to achieve an effective amount of the IL15. The term effective amount is defined as any amount necessary to produce a desired physiologic response. Effective amounts and schedules for administering the ligand may be determined empirically by one skilled in the art based on the amount of resulting IL15, the activity of the IL15, or based on one or more signs of the effect of the IL15 activity. The ranges for administration of the ligand range from zero to a saturating dose and the resulting IL15 activity ranges from a basal level in the absence of ligand to a maximum level in the presence of a saturating amount of ligand. In some embodiments, the method comprises contacting the cell with a selected amount of ligand, wherein the selected amount of ligand results in a selected activity level of the IL15 payload. In certain embodiments, the method comprises alternatively contacting the cell with varying selected amounts of ligand, to achieve varying selected activity levels ranging from the basal level to the maximum level. Optionally with a sufficient dynamic range that allows for the desired dose-response to the ligand and concomitant activity range for the payload (e.g., for a given ligand and payload, the range of difference in off-state and maximum payload activity would result from at least a 10-fold range of ligand). This sufficient dynamic range allows for fine tuning and a dose response curve that is not unacceptably steep.

The ligand can be delivered to achieve continuous or intermittent IL15 payload activity. Continuous payload activity may be a substantially consistent level of activity, or the level of activity may be modulated. Intermittent activity, between the off-state and on-state includes modulating activity between the off-state and a substantially consistent on-state, or between the off-state and varying on-state activity levels. A higher dose or longer duration of administration of the ligand is administered when more activity of the IL15 payload is desired, and reduction or elimination of the ligand dose is chosen when less activity is desired. The dosage or frequency of the administration of the ligand and the resulting amount and activity of the IL15 payload should not be so large as to cause unacceptable adverse side effects and will vary with the age, condition, sex, type of cancer being treated, the extent of the condition, and whether other therapeutic agents are included in the treatment regimen. Guidance can be found in the literature for appropriate dosages for given classes of ligands.

In some embodiments, the TILs modified with mbIL15 or with regulatable mbIL15 (i.e., operably linked to DRD) can be administered in combination with one or more immune checkpoint regulators. Checkpoint inhibitors include antibodies that target PD-1 or inhibit the binding of PD-1 to PD-L1, including, but are not limited to, nivolumab (BMS-936558, Bristol-Myers Squibb; Opdivo®), pembrolizumab (lambrolizumab, MK03475 or MK-3475, Merck; Keytruda®), humanized anti-PD-1 antibody JS001 (ShangHai JunShi), monoclonal anti-PD-1 antibody TSR-042 (Tesaro, Inc.), Pidilizumab (anti-PD-1 mAb CT-011, Medivation), anti-PD-1 monoclonal Antibody BGB-A317 (BeiGene), and/or anti-PD-1 antibody SHR-1210 (ShangHai HengRui), human monoclonal antibody REGN2810 (Regeneron), human monoclonal antibody MDX-1106 (Bristol-Myers Squibb), and/or humanized anti-PD-1 IgG4 antibody PDR001 (Novartis).

The recipient subject is optionally monitored for the outcome of the treatment. Thus, for example, the number of malignant cells in a sample, the circulating tumor DNA in a sample, or the size of a solid tumor upon imaging can be detected. If the desired end point is achieved (e.g., showing successful treatment of cancer), the ligand can be reduced or discontinued so as to reduce or eliminate the IL15. Similarly, if the subject develops a cytokine storm, an allergic reaction, or other adverse effect from the IL15, the ligand can be reduced or discontinued.

### Allogeneic PDX Model

Provided herein is a method for assessing the efficacy of allogeneic TILs engineered to express IL15 in treating a tumor of a recipient subject. The method includes implanting tumor cells or tumor fragments from the recipient subject into an immunodeficient or humanized starter animal under conditions for growth of a starter tumor. When the starter tumor reaches a selected size, tumor cells from the starter tumor are implanted into one or more immunodeficient or humanized test animals. Again, the tumor is allowed to grow in the test animal to a selected size and allogenic TILs are administered to the test animal. The allogenic TILs can be administered once or, in certain cases, multiple times. The test animal is then evaluated for efficacy of the allogeneic TILs in treating the tumor in the test animal. If the allogenic TILs have a DRD operably linked to the mbIL15, then the test animal is also treated with the effective amount of the ligand as described herein. The TIL treatment can be provided without the need for exogenous cytokines such as interleukins like IL2.

Efficacy can be evaluated in a number of ways, for example, by measuring the size of the test tumor (e.g., before and after administration of the TILs or compared to an untreated control animal), by detecting TIL infiltration into the test tumor after administration (e.g., as compared to an untreated control animal), by imaging of the test animal (e.g., over the course of treatment or as compared to an untreated test animal), by comparing survival time of the treated and untreated test animals. For example, no tumor growth in the treated animal or reduction in tumor size in the test animal or less tumor growth in the treated animal as compared to the tumor growth in an untreated animal can indicate efficacy. Similarly, a treated (receiving TILs) and an untreated test animal (receiving no TILs) can be sacrificed at the end of a selected period and TILs can be stained immunohistochemically in tumor sections as described herein to determine TIL infiltration. More TIL infiltration is also indicative of efficacy. Imaging (e.g., Xray or CT scanning) can be used to measure tumor growth over time or as compared to a control animal receiving no TILs or receiving regulated TILs without ligand.

The starter animals and test animals can be mice. For example, humanized mice, athymic nude mice, severely compromised immune deficient (SCID) mice, NOD-SCID mice, or recombination-activating gene 2 (Rag2)-knockout mice. NOD-SCID mice include NOD/SCID mice with IL2rg mutations, such as NOD.Cg-Prkdescid Il2rgtm 1Wjl (NSG™) and NODShi.Cg-Prkdcscid Il2rgtm 1 Sug (NOG®) mice.

The TILs administered to the test animals are optionally an expanded population of TILs derived from a tumor sample of a donor subject. Briefly, allogeneic TILs can be modified to express mbIL15 (regulated or constitutive) and expanded in vitro according to the methods taught herein prior to treatment of the test animal with the TIL population. The expansion step comprises expanding the modified TILs in the presence of K562 feeder cells, wherein the K562 feeder cells are modified to express 41BB ligand (41BBL) and IL21 (e.g., mbIL21). Expansion occurs without the need for IL2.

### Definitions

The terms about and approximate, when used to refer to a measurable value such as an amount, concentration, dose, time, temperature, activity, level, number, frequency, percentage, dimension, size, weight, position, length and the like, is meant to account for variations due to experimental error, which could encompass variations of ±15%, ±10%, ±5%, ±1%, ±0.5%, or even ±0.1% of the specified amount, concentration, dose, time, temperature, activity, level, number, frequency, percentage, dimension, size, weight, position, length and the like. All measurements or numbers are implicitly understood to be modified by the word about, even if the measurement or number is not explicitly modified by the word about. In instances in which the terms about and approximate are used in connection with the location or position of regions within a reference polypeptide, these terms encompass variations of ± up to 20 amino acid residues, ± up to 15 amino acid residues, ± up to 10 amino acid residues, ± up to 5 amino acid residues, ± up to 4 amino acid residues, ± up to 3 amino acid residues, ± up to 2 amino acid residues, or even ±1 amino acid residue.

As used herein, operably linked means that, in the presence of a paired ligand, the DRD is linked to the IL15 directly or indirectly so as to alter a measurable characteristic of the IL15 (e.g., alters the level of activity of the IL15 as compared to the level of activity in the absence of the paired ligand). In some embodiments, the measured level of amount and/or activity of the IL15 increases in the presence of an effective amount of ligand as compared to the measured level of expression or activity in the absence of ligand. An effective amount the ligand means the amount of ligand needed to see an increase in the measure of the amount or activity of the IL15. In some embodiments, the effective amount is not so great as to produce unacceptable toxicity or off-target effects. Optionally, the measurable characteristic is a therapeutic outcome, an amount of the payload in a sample, or a biological activity level of the payload (for which measuring the amount of payload can serve as a proxy.

Wherever the phrase linked or bound or the like is used, the phrase directly or indirectly is understood to follow unless explicitly stated otherwise or nonsensical in context. Thus, reference to a DRD linked to, bound to, or associated with mbIL15 means in each case that a DRD is directly or indirectly linked to a IL15.

As used herein the terms survival of TILs and persistence pf TILs are used interchangeably. Survival is determined based on a persistent effect of the TILs.

As used herein, expansion is used to refer to a functional increase in cell number that occurs during a functional REP. A functional REP results in an expanded cell population that provides sufficient cell numbers for therapeutic use. An unsuccessful REP, on the other hand, would result in the absence of a functional fold increase in cell number. Unexpanded cells include pre-REP cells and those that have not undergone a functional expansion in REP as compared to an expanded cell population. A non-functional expansion incudes expansion of 10% or less of an expanded cell population. For example, a TIL population that expanded 100-fold in a given time period can be compared to an unexpanded population that expanded only 10-fold or less. Thus, as used herein an expanded cell or expanded cell population refers to a cell or population of cells that has undergone a functional REP. An unexpanded cell or population of cells refers to a cell or population of cells pre-REP or subsequent to a REP that failed to result in functional expansion of the population of cells. By way of example, certain modified TILs will expand on modified K562 feeder cells but the fold expansion on PBMCs will be less than 10% of the fold expansion on modified K562 feeder cells. Thus, the unexpanded TILS can be modified TILs pre-REP or modified TILs following REP on PBMCs.

The term identity as known in the art, refers to a relationship between two or more sequences, as determined by comparing the sequences. In the art, identity also means the degree of sequence relatedness between sequences, as determined by the number of matches between strings of two or more residues (amino acid or nucleic acid). Identity measures the percent of identical matches between two or more sequences with gap alignments (if any) addressed by a particular mathematical model or computer program (i.e., algorithms). Identity of related sequences can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Generally, variants of a particular polynucleotide or polypeptide of the disclosure will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, Thomas L. Madren, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.)

The term feeder cell as used herein refers to cells that support the expansion of TILs in culture, such as by secreting into the cell culture or presenting on the feeder cell membrane growth or survival factors. In some embodiments, feeder cells are growth arrested (i.e., replication incompetent).

As used herein, subject and patient are used synonymously and are not meant to be limited to human subjects or patients.

Treatment, as used herein refers to a reduction or delay in one or more signs or symptoms of the cancer. As an example, a favorable change of at least 10% in a measurable parameter of disease, and preferably at least 20%, 30%, 40%, 50% or more can be indicative of effective treatment. Thus, efficacy of treatment or amelioration of disease can be assessed, for example by measuring disease progression, disease remission, symptom severity, reduction in pain, quality of life, dose of a medication required to sustain a treatment effect, level of a disease marker, or any other measurable parameter appropriate for a given disease being treated or targeted for treating. In connection with the administration of compositions of the present disclosure, effective amount for treatment of cancer, indicates that administration in a clinically appropriate manner results in a beneficial effect for at least a statistically significant fraction of patients, such as an improvement of symptoms, a cure, a reduction in disease load, reduction in tumor mass or cell numbers, extension of life, improvement in quality of life, or other effect generally recognized as positive by medical doctors familiar with treating the particular type of cancer.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

The details of one or more embodiments of the present disclosure are set forth in the description and accompanying drawings. It is to be understood that other embodiments may be utilized and structural or process changes made without departing from the scope of the disclosure. In other words, illustrative embodiments and aspects are described. But it will be appreciated that, in the development of any such actual embodiment, numerous implementation-specific decisions may be made to achieve the developer's specific goals, such as compliance with clinically relevant constraints, which may vary from one implementation to another. Moreover, it will be appreciated that such development effort might be complex and time-consuming but would nevertheless be a routine undertaking for those of ordinary skill in the art having the benefit of this disclosure.

Publications cited herein and the material for which they are cited are hereby specifically incorporated by reference in their entireties.

The examples below are intended to further illustrate certain aspects of the methods and compositions described herein, and are not intended to limit the scope of the claims.

### EXAMPLES

### Example 1: Isolation and Expansion of TIL from Patient Tumor Samples (Pre-REP Culture)

Melanoma and head and neck tumor samples were obtained from Cooperative Human Tissue Network. Tumor samples were cut into 1-3 mm fragments in Hanks' Balanced Salt Solution (HBSS) buffer and fragments were placed in multi-well plates at 1 fragment/well in 2 mL of TIL culture media (RPMI-1640 supplemented with GlutaMAX (Thermo Fisher), 1% Penicillin/Streptomycin, 1 mM Sodium Pyruvate, 1% HEPES, 50 μM 2-Mercaptoethanol (Invitrogen) and 10% heat-inactivated human AB serum (Valley Bio)) containing 6000 IU/mL IL2 (Peprotech) and 0.1 mg/mL Normocin (InvivoGen). Half of the media was replaced with fresh media containing IL2 starting on day 5 and cells were split into multiple wells or flasks as they became confluent for a duration of 3 weeks. This culture process is referred to as pre-rapid expansion protocol (pre-REP). After pre-REP, TILs were aliquoted, frozen in cell freezing media (Bambanker, Bulldog Bio or Cryostor-10, STEMCELL Technologies) and stored long-term in liquid nitrogen.

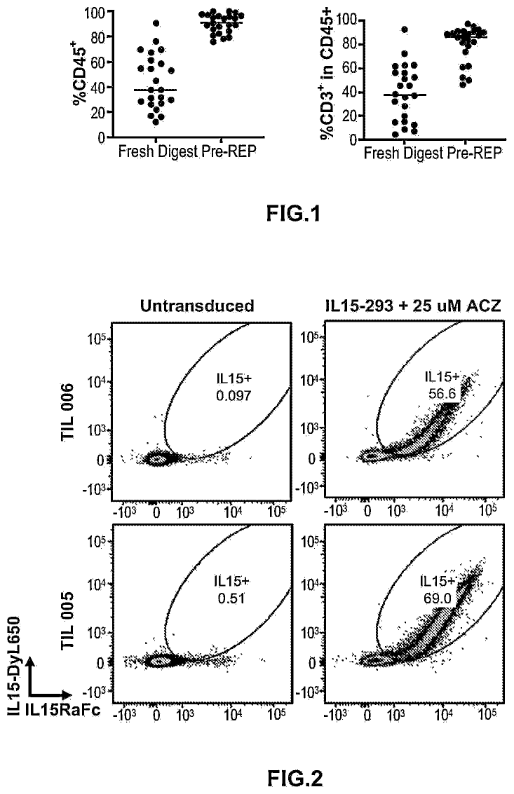

In order to determine the change in frequency of T cells before and after pre-REP culture, a portion of tumor fragments were digested with collagenase and DNase I to generate single cell suspension prior to the pre-REP culture and compared to cells obtained after the pre-REP culture. Frequency of T cells were analyzed by flow cytometry using fluorochrome conjugated anti-CD45 and anti-CD3 antibodies. As shown in FIG. 1, nearly half of the cells (44.29±21.67%) in the pre-culture tumor cell suspension were CD45+ and among these only approximately 40% (˜39.85±23.69%) were CD3+ T cells. After 3 weeks of culture in the presence of IL2 (pre-REP), the majority of the cells were CD45+ (90.35±7.28%), indicating an enrichment of hematopoietic cells, and CD3+ (80.64±15.19%), indicating an enrichment of T cells.

TILs from other human tumor types, including melanoma tumors and malignant tumors from breast, lung, kidney, endometrium, liver, pancreas and ovary, were isolated in the same manner as described above.

### Example 2: Generation of K562 Cells Expressing Membrane-Bound IL21 and 4-1BBL

### Membrane-Bound IL21 and 4-1BBL Vector Construct Assembly

The IL21-41BBL-001 insert comprises nucleic acid sequences encoding a leader sequence, membrane-bound IL21 (mbIL21), a P2A sequence and 4-1BBL. The mbIL21 nucleic acid sequence encodes, in order, an IL21 sequence, an IgG hinge, an IgG4 chain, a CD4 transmembrane domain and a Glycine-Serine (GS) linker (see Table 3). OT-IL21-41BBL-001, which comprises the IL21-41BBL-001 insert, was constructed in a pELNS vector (a third-generation self-inactivating lentiviral expression vector) using standard molecular biology techniques. Gene fragments (Gblocks) were inserted into the pELNS vector and placed under the control of the EF1a promoter using Gibson assembly (NEBuilder Hifi). The assembled plasmid was transformed into E. coli (NEB stable) for amplification and sequence was confirmed before proceeding with virus production.

TABLE 3
Components of a mbIL21-41BBL Construct

| | | NA | | AA |
|---|---|---|---|---|
| | | SEQ | | SEQ |
| Description | | ID | | ID |
| of Domain | NA Sequence | NO | AA Sequence | NO |
| GM-CSF | ATGTGGCTGCAGTCTCTGCTCCTCTTGGGGACTG | 32 | MWLQSLLLLGTV | 33 |
| Signal | TCGCCTGTTCTATTTCA | | ACSIS | |
| Peptide | | | | |
| IL21 | CAAGGACAGGATCGACATATGATTCGGATGCGC | 34 | QGQDRHMIRMR | 35 |
| | CAACTGATAGATATAGTCGATCAACTCAAGAAT | | QLIDIVDQLKNYV | |
| | TATGTGAATGACTTGGTCCCTGAGTTTCTGCCGG | | NDLVPEFLPAPED | |
| | CTCCAGAGGACGTTGAAACAAACTGTGAATGGT | | VETNCEWSAFSC | |
| | CAGCGTTTTCATGTTTTCAAAAGGCACAGCTCAA | | FQKAQLKSANTG | |
| | GTCCGCCAATACAGGCAATAACGAGCGGATTAT | | NNERIINVSIKKL | |
| | AAATGTCTCAATTAAAAAGCTCAAGCGCAAACC | | KRKPPSTNAGRR | |
| | CCCTTCAACGAATGCTGGTCGCCGCCAGAAACA | | QKHRLTCPSCDS | |
| | CAGGTTGACCTGTCCCTCCTGTGACTCATACGAG | | YEKKPPKEFLERF | |
| | AAGAAACCTCCCAAGGAATTTCTCGAACGCTTT | | KSLLQKMIHQHL | |
| | AAGTCACTCTTGCAGAAGATGATTCATCAGCACT | | SSRTHGSEDS | |
| | TGAGTAGCCGGACACATGGTTCAGAGGATAGT | | | |
| IgG Hinge | GAGTCTAAGTATGGCCCACCGTGTCCCCCCTGCC | 36 | ESKYGPPCPPCP | 37 |
| (S228P) | CA | | | |
| IgG4 Chain | GCACCTGAGTTCCTCGGAGGCCCCTCTGTATTCC | 38 | APEFLGGPSVFLF | 39 |
| | TGTTTCCCCCAAAGCCCAAGGATACTCTTATGAT | | PPKPKDTLMISRT | |
| | CTCACGCACTCCGGAAGTAACCTGCGTGGTGGT | | PEVTCVVVDVSQ | |
| | GGATGTGAGTCAGGAAGACCCCGAAGTCCAGTT | | EDPEVQFNWYVD | |
| | TAATTGGTACGTGGACGGGGTTGAGGTACATAA | | GVEVHNAKTKPR | |
| | CGCCAAAACGAAACCTCGGGAGGAGCAATTCAA | | EEQFNSTYRVVS | |
| | TTCCACTTACCGGGTTGTATCAGTCCTGACTGTA | | VLTVLHQDWLN | |
| | CTGCATCAAGATTGGCTCAACGGGAAAGAGTAC | | GKEYKCKVSNKG | |
| | AAGTGTAAGGTTAGTAATAAAGGGCTGCCGTCT | | LPSSIEKTISKAKG | |
| | AGTATTGAGAAAACGATCAGTAAGGCTAAAGGG | | QPREPQVYTLPPS | |
| | CAGCCAAGAGAGCCACAAGTATATACCCTGCCA | | QEEMTKNQVSLT | |
| | CCCTCTCAGGAGGAGATGACTAAAAACCAAGTG | | CLVKGFYPSDIAV | |
| | TCACTGACCTGCCTTGTTAAGGGTTTTTACCCAT | | EWESNGQPENNY | |
| | CTGATATAGCAGTAGAGTGGGAATCCAATGGAC | | KTTPPVLDSDGSF | |
| | AGCCAGAGAACAATTATAAGACTACACCTCCCG | | FLYSRLTVDKSR | |
| | TCCTTGATAGTGACGGCTCCTTCTTCTTGTATTCT | | WQEGNVFSCSVM | |
| | CGACTTACAGTTGATAAGTCCCGCTGGCAGGAG | | HEALHNHYTQKS | |
| | GGTAATGTCTTTAGCTGCAGTGTAATGCACGAA | | LSLSLGK | |
| | GCTCTTCATAATCACTACACACAAAAATCATTGA | | | |
| | GCCTGTCTCTGGGAAAG | | | |
| CD4 Trans- | ATGGCCTTGATTGTGCTCGGCGGAGTTGCAGGCC | 40 | MALIVLGGVAGL | 41 |
| membrane | TGCTCCTTTTTATTGGACTCGGAATATTTTTC | | LLFIGLGIFF | |
| Linker | GGATCTGGA | 42 | GSG | 43 |
| P2A | GCTACTAACTTCAGCCTGCTGAAGCAGGCTGGA | 44 | ATNFSLLKQAGD | 45 |
| | GACGTGGAGGAGAACCCTGGACCT | | VEENPGP | |
| 4-1BBL | ATGGAGTACGCTAGTGATGCGTCCTTGGACCCC | 46 | MEYASDASLDPE | 47 |
| | GAGGCGCCATGGCCACCGGCCCCGCGAGCTCGA | | APWPPAPRARAC | |
| | GCCTGTCGAGTGCTGCCATGGGCTCTGGTCGCTG | | RVLPWALVAGLL | |
| | GGTTGCTCCTCCTTCTGCTTTTGGCCGCGGCTTGT | | LLLLLAAACAVF | |
| | GCAGTGTTTCTTGCTTGCCCGTGGGCAGTTAGCG | | LACPWAVSGARA | |
| | GTGCTCGCGCATCTCCCGGAAGCGCGGCGAGTC | | SPGSAASPRLREG | |
| | CTCGACTCAGGGAAGGTCCGGAGCTGAGCCCAG | | PELSPDDPAGLLD | |
| | ATGACCCCGCCGGTTTGCTGGACCTCCGCCAAG | | LRQGMFAQLVAQ | |
| | GAATGTTCGCTCAACTCGTTGCGCAAAACGTACT | | NVLLIDGPLSWYS | |
| | TCTTATAGACGGCCCTCTTAGTTGGTACAGTGAC | | DPGLAGVSLTGG | |
| | CCAGGATTGGCTGGCGTTAGTTTGACAGGCGGA | | LSYKEDTKELVV | |
| | CTCAGTTACAAGGAGGATACTAAGGAACTGGTA | | AKAGVYYVFFQL | |
| | GTCGCTAAGGCTGGGGTATACTACGTGTTCTTTC | | ELRRVVAGEGSG | |
| | AACTCGAACTGAGAAGGGTGGTTGCGGGAGAAG | | SVSLALHLQPLRS | |
| | GATCTGGAAGTGTATCTCTCGCCCTGCACCTCCA | | AAGAAALALTVD | |
| | ACCCCTCAGAAGTGCCGCCGGAGCGGCCGCCCT | | LPPASSEARNSAF | |
| | TGCCCTTACTGTCGACCTGCCCCCGGCTTCTTCA | | GFQGRLLHLSAG | |
| | GAAGCGCGAAATAGTGCATTCGGCTTCCAGGGG | | QRLGVHLHTEAR | |
| | CGCCTTTTGCACTTGAGCGCTGGACAGCGCCTCG | | ARHAWQLTQGA | |
| | GGGTCCACCTCCACACGGAAGCGCGGGCGAGGC | | TVLGLFRVTPEIP | |
| | ACGCTTGGCAACTCACACAAGGTGCGACGGTTC | | AGLPSPRSE | |
| | TCGGCTTGTTTAGGGTTACGCCTGAGATACCGGC | | | |
| | TGGCCTCCCATCTCCAAGATCCGAG | | | |
| Linker | GGATCC | 25 | GS | 24 |
| Stop Codon | TAA | 48 | N/A | |
| IL21-41BBL | ATGTGGCTGCAGTCTCTGCTCCTCTTGGGGACTG | 49 | MWLQSLLLLGTV | 50 |
| insert | TCGCCTGTTCTATTTCACAAGGACAGGATCGACA | | ACSISQGQDRHMI | |
| | TATGATTCGGATGCGCCAACTGATAGATATAGTC | | RMRQLIDIVDQL | |
| | GATCAACTCAAGAATTATGTGAATGACTTGGTCC | | KNYVNDLVPEFL | |
| | CTGAGTTTCTGCCGGCTCCAGAGGACGTTGAAA | | PAPEDVETNCEW | |
| | CAAACTGTGAATGGTCAGCGTTTTCATGTTTTCA | | SAFSCFQKAQLKS | |
| | AAAGGCACAGCTCAAGTCCGCCAATACAGGCAA | | ANTGNNERIINVS | |
| | TAACGAGCGGATTATAAATGTCTCAATTAAAAA | | IKKLKRKPPSTNA | |
| | GCTCAAGCGCAAACCCCCTTCAACGAATGCTGG | | GRRQKHRLTCPS | |
| | TCGCCGCCAGAAACACAGGTTGACCTGTCCCTCC | | CDSYEKKPPKEFL | |
| | TGTGACTCATACGAGAAGAAACCTCCCAAGGAA | | ERFKSLLQKMIH | |
| | TTTCTCGAACGCTTTAAGTCACTCTTGCAGAAGA | | QHLSSRTHGSEDS | |
| | TGATTCATCAGCACTTGAGTAGCCGGACACATG | | ESKYGPPCPPCPA | |
| | GTTCAGAGGATAGTGAGTCTAAGTATGGCCCAC | | PEFLGGPSVFLFP | |
| | CGTGTCCCCCCTGCCCAGCACCTGAGTTCCTCGG | | PKPKDTLMISRTP | |
| | AGGCCCCTCTGTATTCCTGTTTCCCCCAAAGCCC | | EVTCVVVDVSQE | |
| | AAGGATACTCTTATGATCTCACGCACTCCGGAA | | DPEVQFNWYVD | |
| | GTAACCTGCGTGGTGGTGGATGTGAGTCAGGAA | | GVEVHNAKTKPR | |
| | GACCCCGAAGTCCAGTTTAATTGGTACGTGGAC | | EEQFNSTYRVVS | |
| | GGGGTTGAGGTACATAACGCCAAAACGAAACCT | | VLTVLHQDWLN | |
| | CGGGAGGAGCAATTCAATTCCACTTACCGGGTT | | GKEYKCKVSNKG | |
| | GTATCAGTCCTGACTGTACTGCATCAAGATTGGC | | LPSSIEKTISKAKG | |
| | TCAACGGGAAAGAGTACAAGTGTAAGGTTAGTA | | QPREPQVYTLPPS | |
| | ATAAAGGGCTGCCGTCTAGTATTGAGAAAACGA | | QEEMTKNQVSLT | |
| | TCAGTAAGGCTAAAGGGCAGCCAAGAGAGCCAC | | CLVKGFYPSDIAV | |
| | AAGTATATACCCTGCCACCCTCTCAGGAGGAGA | | EWESNGQPENNY | |
| | TGACTAAAAACCAAGTGTCACTGACCTGCCTTGT | | KTTPPVLDSDGSF | |
| | TAAGGGTTTTTACCCATCTGATATAGCAGTAGAG | | FLYSRLTVDKSR | |
| | TGGGAATCCAATGGACAGCCAGAGAACAATTAT | | WQEGNVFSCSVM | |
| | AAGACTACACCTCCCGTCCTTGATAGTGACGGCT | | HEALHNHYTQKS | |
| | CCTTCTTCTTGTATTCTCGACTTACAGTTGATAA | | LSLSLGKMALIVL | |
| | GTCCCGCTGGCAGGAGGGTAATGTCTTTAGCTGC | | GGVAGLLLFIGLG | |
| | AGTGTAATGCACGAAGCTCTTCATAATCACTACA | | IFFGSGATNFSLL | |
| | CACAAAAATCATTGAGCCTGTCTCTGGGAAAGA | | KQAGDVEENPGP | |
| | TGGCCTTGATTGTGCTCGGCGGAGTTGCAGGCCT | | MEYASDASLDPE | |
| | GCTCCTTTTTATTGGACTCGGAATATTTTTCGGA | | APWPPAPRARAC | |
| | TCTGGAgctactaacttcagcctgctgaagcaggct | | RVLPWALVAGLL | |
| | ggagacgtggaggagaaccctggacctATGGAGTAC | | LLLLLAAACAVF | |
| | GCTAGTGATGCGTCCTTGGACCCCGAGGCGCCATGG | | LACPWAVSGARA | |
| | CCACCGGCCCCGCGAGCTCGAGCCTGTCGAGTGCTG | | SPGSAASPRLREG | |
| | CCATGGGCTCTGGTCGCTGGGTTGCTCCTCCTTCTGC | | PELSPDDPAGLLD | |
| | TTTTGGCCGCGGCTTGTGCAGTGTTTCTTGCTTGCCC | | LRQGMFAQLVAQ | |
| | GTGGGCAGTTAGCGGTGCTCGCGCATCTCCCGGAAGC | | NVLLIDGPLSWYS | |
| | GCGGCGAGTCCTCGACTCAGGGAAGGTCCGGAGC | | DPGLAGVSLTGG | |
| | TGAGCCCAGATGACCCCGCCGGTTTGCTGGACCT | | LSYKEDTKELVV | |
| | CCGCCAAGGAATGTTCGCTCAACTCGTTGCGCA | | AKAGVYYVFFQL | |
| | AAACGTACTTCTTATAGACGGCCCTCTTAGTTGG | | ELRRVVAGEGSG | |
| | TACAGTGACCCAGGATTGGCTGGCGTTAGTTTGA | | SVSLALHLQPLRS | |
| | CAGGCGGACTCAGTTACAAGGAGGATACTAAGG | | AAGAAALALTVD | |
| | AACTGGTAGTCGCTAAGGCTGGGGTATACTACG | | LPPASSEARNSAF | |
| | TGTTCTTTCAACTCGAACTGAGAAGGGTGGTTGC | | GFQGRLLHLSAG | |
| | GGGAGAAGGATCTGGAAGTGTATCTCTCGCCCT | | QRLGVHLHTEAR | |
| | GCACCTCCAACCCCTCAGAAGTGCCGCCGGAGC | | ARHAWQLTQGA | |
| | GGCCGCCCTTGCCCTTACTGTCGACCTGCCCCCG | | TVLGLFRVTPEIP | |
| | GCTTCTTCAGAAGCGCGAAATAGTGCATTCGGCT | | AGLPSPRSEGS* | |
| | TCCAGGGGCGCCTTTTGCACTTGAGCGCTGGACA | | | |
| | GCGCCTCGGGGTCCACCTCCACACGGAAGCGCG | | | |
| | GGCGAGGCACGCTTGGCAACTCACACAAGGTGC | | | |
| | GACGGTTCTCGGCTTGTTTAGGGTTACGCCTGAG | | | |
| | ATACCGGCTGGCCTCCCATCTCCAAGATCCGAG | | | |
| | GGATCCtaa | | | |
| OT-IL21- | gcgcgctcactggccgtcgttttacaacgtcgtgactgggaaaacc | 51 | N/A | |
| 41BBL-001 | ctggcgttacccaacttaatcgccttgcagcacatccccctttcgc | | | |
| | cagctggcgtaatagcgaagaggcccgcaccgatcgcccttcccaa | | | |
| | cagttgcgcagcctgaatggcgaatgggacgcgccctgtagcggcg | | | |
| | cattaagcgcggcgggtgtggtggttacgcgcagcgtgaccgctac | | | |
| | acttgccagcgccctagcgcccgctcctttcgctttcttcccttcc | | | |
| | tttctcgccacgttcgccggctttccccgtcaagctctaaatcggg | | | |
| | ggctccctttagggttccgatttagtgctttacggcacctcgaccc | | | |
| | caaaaaacttgattagggtgatggttcacgtagtgggccatcgccc | | | |
| | tgatagacggtttttcgccctttgacgttggagtccacgttctttaa | | | |
| | tagtggactcttgttccaaactggaacaacactcaaccctatctcg | | | |
| | gtctattcttttgatttataagggattttgccgatttcggcctattg | | | |
| | gttaaaaaatgagctgatttaacaaaaatttaacgcgaattttaac | | | |
| | aaaatattaacgcttacaatttaggtggcacttttcggggaaatgt | | | |
| | gcgcggaacccctatttgtttatttttctaaatacattcaaatatg | | | |
| | tatccgctcatgagacaataaccctgataaatgcttcaataatatt | | | |
| | gaaaaaggaagagtatgagtattcaacatttccgtgtcgcccttat | | | |
| | tcccttttttgcggcattttgccttcctgtttttgctcacccagaa | | | |
| | acgctggtgaaagtaaaagatgctgaagatcagttgggtgcacgag | | | |
| | tgggttacatcgaactggatctcaacagcggtaagatccttgagag | | | |
| | ttttcgccccgaagaacgttttccaatgatgagcacttttaaagtt | | | |
| | ctgctatgtggcgcggtattatcccgtattgacgccgggcaagag | | | |
| | caactcggtcgccgcatacactattctcagaatgacttggttgagt | | | |
| | actcaccagtcacagaaaagcatcttacggatggcatgacagtaag | | | |
| | agaattatgcagtgctgccataaccatgagtgataacactgcggcc | | | |
| | aacttacttctgacaacgatcggaggaccgaaggagctaaccgctt | | | |
| | ttttgcacaacatgggggatcatgtaactcgccttgatcgttggga | | | |
| | accggagctgaatgaagccataccaaacgacgagcgtgacaccacg | | | |
| | atgcctgtagcaatggcaacaacgttgcgcaaactattaactggcga | | | |
| | actacttactctagcttcccggcaacaattaatagactggatggag | | | |
| | gcggataaagttgcaggaccacttctgcgctcggcccttccggctg | | | |
| | gctggtttattgctgataaatctggagccggtgagcgtgggtctcgc | | | |
| | ggtatcattgcagcactggggccagatggtaagccctcccgtatcgt | | | |
| | agttatctacacgacggggagtcaggcaactatggatgaacgaaata | | | |
| | gacagatcgctgagataggtgcctcactgattaagcattggtaactg | | | |
| | tcagaccaagtttactcatatatactttagattgatttaaaacttca | | | |
| | tttttaatttaaaaggatctaggtgaagatcctttttgataatctca | | | |
| | tgaccaaaatcccttaacgtgagttttcgttccactgagcgtcagac | | | |
| | cccgtagaaaagatcaaaggatcttcttgagatcctttttttctgcg | | | |
| | cgtaatctgctgcttgcaaacaaaaaaaccaccgctaccagcggtgg | | | |
| | tttgtttgccggatcaagagctaccaactctttttccgaaggtaact | | | |
| | ggcttcagcagagcgcagataccaaatactgttcttctagtgtagcc | | | |
| | gtagttaggccaccacttcaagaactctgtagcaccgcctacatacc | | | |
| | tcgctctgctaatcctgttaccagtggctgctgccagtggcgataag | | | |
| | tcgtgtcttaccgggttggactcaagacgatagttaccggataaggc | | | |
| | gcagcggtcgggctgaacggggggttcgtgcacacagcccagcttgg | | | |
| | agcgaacgacctacaccgaactgagatacctacagcgtgagctatga | | | |
| | gaaagcgccacgcttcccgaagggagaaaggcggacaggtatccggt | | | |
| | aagcggcagggtcggaacaggagagcgcacgagggagcttccagggg | | | |
| | gaaacgcctggtatctttatagtcctgtcgggtttcgccacctctga | | | |
| | cttgagcgtcgatttttgtgatgctcgtcaggggggcggagcctatg | | | |
| | gaaaaacgccagcaacgcggcctttttacggttcctggccttttgct | | | |
| | ggccttttgctcacatgttctttcctgcgttatcccctgattctgtg | | | |
| | gataaccgtattaccgcctttgagtgagctgataccgctcgccgcag | | | |
| | ccgaacgaccgagcgcagcgagtcagtgagcgaggaagcggaagagc | | | |
| | gcccaatacgcaaaccgcctctccccgcgcgttggccgattcattaa | | | |
| | tgcagctggcacgacaggtttcccgactggaaagcgggcagtgagcg | | | |
| | caacgcaattaatgtgagttagctcactcattaggcaccccaggctt | | | |
| | tacactttatgcttccggctcgtatgttgtgtggaattgtgagcgga | | | |
| | taacaatttcacacaggaaacagctatgaccatgattacgccaagcg | | | |
| | cgcaattaaccctcactaaagggaacaaaagctggagctgcaagctt | | | |
| | aatgtagtcttatgcaatactcttgtagtcttgcaacatggtaacga | | | |
| | tgagttagcaacatgccttacaaggagagaaaaagcaccgtgcatgc | | | |
| | cgattggtggaagtaaggtggtacgatcgtgccttattaggaaggca | | | |
| | acagacgggtctgacatggattggacgaaccactgaattgccgcatt | | | |
| | gcagagatattgtatttaagtgcctagctcgatacataaacgggtct | | | |
| | ctctggttagaccagatctgagcctgggagctctctggctaactagg | | | |
| | gaacccactgcttaagcctcaataaagcttgccttgagtgcttcaag | | | |
| | tagtgtgtgcccgtctgttgtgtgactctggtaactagagatccctc | | | |
| | agacccttttagtcagtgtggaaaatctctagcagtggcgcccgaac | | | |
| | agggacttgaaagcgaaagggaaaccagaggagctctctcgacgcag | | | |
| | gactcggcttgctgaagcgcgcacggcaagaggcgaggggcggcgac | | | |
| | tggtgagtacgccaaaaattttgactagcggaggctagaaggagaga | | | |
| | gatgggtgcgagagcgtcagtattaagcgggggagaattagatcgcg | | | |
| | atgggaaaaaattcggttaaggccagggggaaagaaaaaatataaat | | | |
| | taaaacatatagtatgggcaagcagggagctagaacgattcgcagtt | | | |
| | aatcctggcctgttagaaacatcagaaggctgtagacaaatactggg | | | |
| | acagctacaaccatcccttcagacaggatcagaagaacttagatcat | | | |
| | tatataatacagtagcaaccctctattgtgtgcatcaaaggatagag | | | |
| | ataaaagacaccaaggaagctttagacaagatagaggaagagcaaaa | | | |
| | caaaagtaagaccaccgcacagcaagcggccgctgatcttcagacct | | | |
| | ggaggaggagatatgagggacaattggagaagtgaattatataaata | | | |
| | taaagtagtaaaaattgaaccattaggagtagcacccaccaaggcaa | | | |
| | agagaagagtggtgcagagagaaaaaagagcagtgggaataggagct | | | |
| | ttgttccttgggttcttgggagcagcaggaagcactatgggcgcagc | | | |
| | gtcaatgacgctgacggtacaggccagacaattattgtctggtatag | | | |
| | tgcagcagcagaacaatttgctgagggctattgaggcgcaacagcat | | | |
| | ctgttgcaactcacagtctggggcatcaagcagctccaggcaagaat | | | |
| | cctggctgtggaaagatacctaaaggatcaacagctcctggggattt | | | |
| | ggggttgctctggaaaactcatttgcaccactgctgtgccttggaat | | | |
| | gctagttggagtaataaatctctggaacagatttggaatcacacgac | | | |
| | ctggatggagtgggacagagaaattaacaattacacaagcttaatac | | | |
| | actccttaattgaagaatcgcaaaaccagcaagaaaagaatgaacaa | | | |
| | gaattattggaattagataaatgggcaagtttgtggaattggtttaa | | | |
| | cataacaaattggctgtggtatataaaattattcataatgatagtag | | | |
| | gaggcttggtaggtttaagaatagtttttgctgtactttctatagtg | | | |
| | aatagagttaggcagggatattcaccattatcgtttcagacccacct | | | |
| | cccaaccccgaggggacccgacaggcccgaaggaatagaagaagaag | | | |
| | gtggagagagagacagagacagatccattcgattagtgaacggatct | | | |
| | cgacggtatcgattagactgtagcccaggaatatggcagctagattg | | | |
| | tacacatttagaaggaaaagttatcttggtagcagttcatgtagcca | | | |
| | gtggatatatagaagcagaagtaattccagcagagacagggcaagaa | | | |
| | acagcatacttcctcttaaaattagcaggaagatggccagtaaaaac | | | |
| | agtacatacagacaatggcagcaatttcaccagtactacagttaagg | | | |
| | ccgcctgttggtgggggggatcaagcaggaatttggcattccctaca | | | |
| | atccccaaagtcaaggagtaatagaatctatgaataaagaattaaag | | | |
| | aaaattataggacaggtaagagatcaggctgaacatcttaagacagc | | | |
| | agtacaaatggcagtattcatccacaattttaaaagaaaagggggga | | | |
| | ttggggggtacagtgcaggggaaagaatagtagacataatagcaaca | | | |
| | gacatacaaactaaagaattacaaaaacaaattacaaaaattcaaaa | | | |
| | ttttcgggtttattacagggacagcagagatccagtttggctgcatt | | | |
| | gatcacgtgaggctccggtgcccgtcagtgggcagagcgcacatcgc | | | |
| | ccacagtccccgagaagttggggggaggggtcggcaattgaaccggt | | | |
| | gcctagagaaggtggcgcggggtaaactgggaaagtgatgtcgtgta | | | |
| | ctggctccgcctttttcccgaggggggggagaaccgtatataagtgc | | | |
| | agtagtcgccgtgaacgttctttttcgcaacgggtttgccgccagaa | | | |
| | cacaggtaagtgccgtgtgtggttcccgcgggcctggcctctttacg | | | |
| | ggttatggcccttgcgtgccttgaattacttccacctggctgcagta | | | |
| | cgtgattcttgatcccgagcttcgggttggaagtggggggagagttc | | | |
| | gaggccttgcgcttaaggagccccttcgcctcgtgcttgagttgagg | | | |
| | cctggcctgggcgctggggccgccgcgtgcgaatctggtggcacctt | | | |
| | cgcgcctgtctcgctgctttcgataagtctctagccatttaaaattt | | | |
| | ttgatgacctgctgcgacgctttttttctggcaagatagtcttgtaa | | | |
| | atgcgggccaagatctgcacactggtatttcggtttttggggccgcg | | | |
| | ggcggcgacggggcccgtgcgtcccagcgcacatgttcggcgaggcg | | | |
| | gggcctgcgagcgcggccaccgagaatcggacgggggtagtctcaag | | | |
| | ctggccggcctgctctggtgcctggcctcgcgccgccgtgtatcgcc | | | |
| | ccgccctgggcggcaaggctggcccggtcggcaccagttgcgtgagc | | | |
| | ggaaagatggccgcttcccggccctgctgcagggagctcaaaatgga | | | |
| | ggacgcggcgctcgggagagcgggcgggtgagtcacccacacaaagg | | | |
| | aaaagggcctttccgtcctcagccgtcgcttcatgtgactccactga | | | |
| | gtaccgggcgccgtccaggcacctcgattagttctcgagcttttgga | | | |
| | gtacgtcgtctttaggttggggggaggggttttatgcgatggagttt | | | |
| | ccccacactgagtgggtggagactgaagttaggccagcttggcactt | | | |
| | gatgtaattctccttggaatttgccctttttgagtttggatcttggt | | | |
| | tcattctcaagcctcagacagtggttcaaagtttttttcttccattt | | | |
| | caggtgtcgtgatctagaggatcACTAGTACCATGTGGCTGCAGTCT | | | |
| | CTGCTCCTCTTGGGGACTGTCGCCTGTTCTATTTCACAAGGACAGGA | | | |
| | TCGACATATGATTCGGATGCGCCAACTGATAGATATAGTCG | | | |
| | ATCAACTCAAGAATTATGTGAATGACTTGGTCCC | | | |
| | TGAGTTTCTGCCGGCTCCAGAGGACGTTGAAAC | | | |
| | AAACTGTGAATGGTCAGCGTTTTCATGTTTTCAA | | | |
| | AAGGCACAGCTCAAGTCCGCCAATACAGGCAAT | | | |
| | AACGAGCGGATTATAAATGTCTCAATTAAAAAG | | | |
| | CTCAAGCGCAAACCCCCTTCAACGAATGCTGGT | | | |
| | CGCCGCCAGAAACACAGGTTGACCTGTCCCTCCT | | | |
| | GTGACTCATACGAGAAGAAACCTCCCAAGGAAT | | | |
| | TTCTCGAACGCTTTAAGTCACTCTTGCAGAAGAT | | | |
| | GATTCATCAGCACTTGAGTAGCCGGACACATGG | | | |
| | TTCAGAGGATAGTGAGTCTAAGTATGGCCCACC | | | |
| | GTGTCCCCCCTGCCCAGCACCTGAGTTCCTCGGA | | | |
| | GGCCCCTCTGTATTCCTGTTTCCCCCAAAGCCCA | | | |
| | AGGATACTCTTATGATCTCACGCACTCCGGAAGT | | | |
| | AACCTGCGTGGTGGTGGATGTGAGTCAGGAAGA | | | |
| | CCCCGAAGTCCAGTTTAATTGGTACGTGGACGG | | | |
| | GGTTGAGGTACATAACGCCAAAACGAAACCTCG | | | |
| | GGAGGAGCAATTCAATTCCACTTACCGGGTTGT | | | |
| | ATCAGTCCTGACTGTACTGCATCAAGATTGGCTC | | | |
| | AACGGGAAAGAGTACAAGTGTAAGGTTAGTAAT | | | |
| | AAAGGGCTGCCGTCTAGTATTGAGAAAACGATC | | | |
| | AGTAAGGCTAAAGGGCAGCCAAGAGAGCCACA | | | |
| | AGTATATACCCTGCCACCCTCTCAGGAGGAGAT | | | |
| | GACTAAAAACCAAGTGTCACTGACCTGCCTTGTT | | | |
| | AAGGGTTTTTACCCATCTGATATAGCAGTAGAGT | | | |
| | GGGAATCCAATGGACAGCCAGAGAACAATTATA | | | |
| | AGACTACACCTCCCGTCCTTGATAGTGACGGCTC | | | |
| | CTTCTTCTTGTATTCTCGACTTACAGTTGATAAGT | | | |
| | CCCGCTGGCAGGAGGGTAATGTCTTTAGCTGCA | | | |
| | GTGTAATGCACGAAGCTCTTCATAATCACTACAC | | | |
| | ACAAAAATCATTGAGCCTGTCTCTGGGAAAGAT | | | |
| | GGCCTTGATTGTGCTCGGCGGAGTTGCAGGCCTG | | | |
| | CTCCTTTTTATTGGACTCGGAATATTTTTCGGATC | | | |
| | TGGAgctactaacttcagcctgctgaagcaggctggagacgtggag | | | |
| | gagaaccctggacctATGGAGTACGCTAGTGATGCGTCCTTG | | | |
| | GACCCCGAGGCGCCATGGCCACCGGCCCCGCGA | | | |
| | GCTCGAGCCTGTCGAGTGCTGCCATGGGCTCTGG | | | |
| | TCGCTGGGTTGCTCCTCCTTCTGCTTTTGGCCGC | | | |
| | GGCTTGTGCAGTGTTTCTTGCTTGCCCGTGGGCA | | | |
| | GTTAGCGGTGCTCGCGCATCTCCCGGAAGCGCG | | | |
| | GCGAGTCCTCGACTCAGGGAAGGTCCGGAGCTG | | | |
| | AGCCCAGATGACCCCGCCGGTTTGCTGGACCTCC | | | |
| | GCCAAGGAATGTTCGCTCAACTCGTTGCGCAAA | | | |
| | ACGTACTTCTTATAGACGGCCCTCTTAGTTGGTA | | | |
| | CAGTGACCCAGGATTGGCTGGCGTTAGTTTGAC | | | |
| | AGGCGGACTCAGTTACAAGGAGGATACTAAGGA | | | |
| | ACTGGTAGTCGCTAAGGCTGGGGTATACTACGT | | | |
| | GTTCTTTCAACTCGAACTGAGAAGGGTGGTTGCG | | | |
| | GGAGAAGGATCTGGAAGTGTATCTCTCGCCCTG | | | |
| | CACCTCCAACCCCTCAGAAGTGCCGCCGGAGCG | | | |
| | GCCGCCCTTGCCCTTACTGTCGACCTGCCCCCGG | | | |
| | CTTCTTCAGAAGCGCGAAATAGTGCATTCGGCTT | | | |
| | CCAGGGGCGCCTTTTGCACTTGAGCGCTGGACA | | | |
| | GCGCCTCGGGGTCCACCTCCACACGGAAGCGCG | | | |
| | GGCGAGGCACGCTTGGCAACTCACACAAGGTGC | | | |
| | GACGGTTCTCGGCTTGTTTAGGGTTACGCCTGAG | | | |
| | ATACCGGCTGGCCTCCCATCTCCAAGATCCGAG | | | |
| | GGATCCtaaATCGGGCTAGCgtcgacaatcaacctctggattac | | | |
| | aaaatttgtgaaagattgactggtattcttaactatgttgctcctt | | | |
| | ttacgctatgtggatacgctgctttaatgcctttgtatcatgctat | | | |
| | tgcttcccgtatggctttcattttctcctccttgtataaatcctgg | | | |
| | ttgctgtctctttatgaggagttgtggcccgttgtcaggcaacgtg | | | |
| | gcgtggtgtgcactgtgtttgctgacgcaacccccactggttgggg | | | |
| | cattgccaccacctgtcagctcctttccgggactttcgctttcccc | | | |
| | ctccctattgccacggcggaactcatcgccgcctgccttgcccgct | | | |
| | gctggacaggggctcggctgttgggcactgacaattccgtggtgttg | | | |
| | tcggggaagctgacgtcctttccatggctgctcgcctgtgttgccac | | | |
| | ctggattctgcgcgggacgtccttctgctacgtcccttcggccctca | | | |
| | atccagcggaccttccttcccgcggcctgctgccggctctgcggcct | | | |
| | cttccgcgtcttcgccttcgccctcagacgagtcggatctccctttg | | | |
| | ggccgcctccccgcctggaattcgagctcggtacctttaagaccaat | | | |
| | gacttacaaggcagctgtagatcttagccactttttaaaagaaaagg | | | |
| | ggggactggaagggctaattcactcccaacgaagacaagatctgctt | | | |
| | tttgcttgtactgggtctctctggttagaccagatctgagcctggga | | | |
| | gctctctggctaactagggaacccactgcttaagcctcaataaagct | | | |
| | tgccttgagtgcttcaagtagtgtgtgcccgtctgttgtgtgactct | | | |
| | ggtaactagagatccctcagacccttttagtcagtgtggaaaatctc | | | |
| | tagcagtagtagttcatgtcatcttattattcagtatttataacttg | | | |
| | caaagaaatgaatatcagagagtgagaggaacttgtttattgcagct | | | |
| | tataatggttacaaataaagcaatagcatcacaaatttcacaaataa | | | |
| | agcatttttttcactgcattctagttgtggtttgtccaaactcatca | | | |
| | atgtatcttatcatgtctggctctagctatcccgcccctaactccg | | | |
| | cccatcccgcccctaactccgcccagttccgcccattctccgcccca | | | |
| | tggctgactaattttttttatttatgcagaggccgaggccgcctcgg | | | |
| | cctctgagctattccagaagtagtgaggaggcttttttggaggccta | | | |
| | gggacgtacccaattcgccctatagtgagtcgtattac | | | |

Table 3 presents the nucleic acid and amino acid sequences for domains of a mbIL21-41BBL construct disclosed herein. OT-IL12-(241-262) and OT-CD19-IL12-(297-316, 319-332) plasmids were each constructed in a pELNS vector (a third-generation self-inactivating lentiviral expression vector) using standard molecular biology techniques. Gene fragments (Gblocks or strings DNA) encoding IL12, Glycine-serine linkers, various hinges, transmembrane domains and cytoplasmic tails were purchased from Integrated DNA Technologies or Thermo-fisher scientific. The gene fragments were inserted into the pELNS vector and placed under the control of the EF1a promoter using Gibson assembly (NEBuilder Hifi). The assembled plasmid was transformed into E. coli (NEB stable) for amplification and sequence confirmed before proceeding with virus production.

### OTLV-IL21-41BBL-001 Lentivirus Production

On the day of transfection, HEK293T cells were seeded in collagen-coated tissue culture flasks with 15×10⁶ cells/flask in a total volume of 20 mL growth media (Dulbecco's Modified Eagle Medium (DMEM), 5% fetal bovine serum (FBS), and 1% penicillin/streptomycin). One hour before transfection, the growth media was replaced with warmed SFM4Transfx-293. Cells were transfected using Lipofectamine 3000 transfection reagent and P3000 enhancer reagent in Opti-MEM media with OT-IL21-41BBL-001 and packaging plasmids pRSV.Rev, pMDLg/pRRE, and pMD2.G (Addgene #122590). Media was replaced 6-8 hours (hr) post-transfection with SFM4Transfx-293. Supernatants containing OTLV-IL21-41BBL-001 were harvested 24 hr post-transfection, fresh media was added, and supernatants were harvested again at 48 hr post-transfection. Viral supernatants were filtered to remove debris and concentrated by ultracentrifugation at 25,000 g for 2 hr at 4° C. The OTLV-IL21-41BBL-001 lentivirus was resuspended, aliquoted, and stored at −80° C.

Transduction of K562 Cells with OTLV-IL21-41BBL-001 Lentivirus

K562 cells were cultured in growth media containing RPMI-1640 with 2 mM L-Glutamine and 10% FBS (complete RPMI, Thermo Fisher). On the day of transfection, K562 cells were seeded in multi-well plates at 1.5×10⁵ cells/well in 500 μL K562 cell growth media. The cells were transduced with OTLV-IL21-41BBL-001 lentivirus and then centrifuged at 800 g for one hour at 32° C. Cells were incubated for 24-48 hours and then assessed for viability and expression of IL21 and 4-1BBL by flow cytometry using antibodies eFluor 780 (Thermo Fisher, 1:1000), 4-1BBL phycoerythrin (1:50), and IL21 allophycocyanin (1:50). The transduced K562 cells were expanded in complete RPMI for 17 days, and subsequently aliquoted, frozen using cell freezing media (Bambanker, Bulldog Bio), and stored in liquid nitrogen long-term. These transduced K562 will be referred to as K562-IL21-41BBL in this document.

The K562-IL21-41BBL cells were irradiated or treated with mitomycin C prior to their use as feeder cells in the TIL REP process. For irradiation, K562-IL21-41BBL cells were taken from fresh cell culture, centrifuged and resuspended in complete RPMI at 5-20×10⁶ cells/mL. Resuspended cells were exposed to 50-200Gy in an X-ray irradiator, following which cells were washed and resuspended at 3×10⁶ cells/mL for immediate use in the REP process. For mitomycin C treatment, the cells were thawed, centrifuged and resuspended in TIL media at 5×10⁶ cells/mL. 10 μg/mL Mitomycin-C was added to the cells and the cells were incubated for 30 minutes at 37° C. The cells were then washed three times with 50 mL TIL media and resuspended at 3×10⁶ cells/mL for immediate use in the REP process.

### Example 3. Transduction of TIL with Lentiviral Vectors

### IL15 Vector Construct Assembly

OT-IL15-292 and OT-IL15-293 (sequences below) were each constructed in a pELNS vector (a third-generation self-inactivating lentiviral expression vector) using standard molecular biology techniques. Gene fragments (Gblocks) encoding codon-optimized IL15, GS linker, B7-1 hinge, transmembrane domain and cytoplasmic tails were purchased from Integrated DNA Technologies, Inc. (IDT, Coralville, Iowa). The gene fragments were inserted into the pELNS vector and placed under the control of the EF1a promoter using Gibson assembly (NEBuilder Hifi). The assembled plasmid was transformed into E. coli (NEB stable) for amplification and sequence confirmed before proceeding with virus production.

Table 1 and Table 2 (provided above) present the nucleic acid and amino acid sequences for components of a constitutive mbIL15 construct (OT-IL15-292) and an ACZ-regulated mbIL15 construct (OT-IL15-293) disclosed herein. Construct OT-IL15-293 comprises a destabilizing domain labeled as CA2 (M1del, L156H) in Table 1.

Table 2 (provided above) also presents the nucleic acid and amino acid sequences of the constitutive IL15 (IL15-292) and ACZ-regulated IL15 (IL15-293) constructs disclosed herein.

### BaEV-Pseudotyped Lentivirus Production

HEK293T cells were seeded on collagen coated tissue culture plates until 70% confluent. Cells were transfected with pELNS transfer vector carrying constitutive (IL15-292) or regulated (IL15-293) IL15 constructs, as well as packaging plasmids pRSV.Rev (Addgene #12253), pMDLg/pRRE (Addgene #12251) and OT-BaEVg-002 (SEQ ID NO: XX) using Lipofectamine 3000 transfection reagent and P3000 enhancer reagent (Thermo Fisher) in Opti-MEM media (Thermo Fisher). Media was replaced 6-8 hours (hr) post-transfection with serum-free media (SFM4Transfx-293, Cytiva). Supernatants containing virus were harvested 24 hr post-transfection, fresh media was added, and supernatants were harvested again at 48 hr post-transfection. Viral supernatants were filtered to remove debris and concentrated by low-speed ultracentrifugation. Virus were resuspended, aliquoted and stored at −80° C.

Rapid Expansion Protocol (REP) and Transduction of TILs with BaEV-Pseudotyped Lentiviral Vectors

TILs generated from a head and neck tumor sample prepared as described in Example 1 were engineered after 3 weeks in the pre-REP culture. TILs were thawed and rested overnight in TIL media with 6000 IU/mL human IL2. TILs were then activated for 24 hr in 24-well plates with anti-CD3/CD28 beads (Dynabeads, Thermo Fisher) at 3:1 bead to TIL ratio or with plate-bound OKT3 at 3 μg/mL (Ultra-LEAF purified anti-human CD3 antibody, Biolegend) and 6000 IU/mL human IL2. RetroNectin (30 μg/mL) was used to coat 96-well non-coated cell culture plates overnight at 4° C. The following day, RetroNectin was removed, the plates were blocked with 2% bovine serum albumin (BSA) in PBS, and the plates were then washed with PBS. BaEV-pseudotyped lentivirus supernatants, prepared as described above, were diluted in TIL media and added in a total volume of 100-200 μL per well for an MOI of 1-4 TU/cell. The plates containing viral vector were centrifuged at 1400 g for 2 hr at 32° C., and the supernatant was then removed. After supernatant removal, 1.5×10⁵ activated TILs were transferred per well with 0-6000 IU/mL IL2 and incubated at 37° C. overnight. Cells were processed similarly without virus addition and used as negative control (“unengineered”). 24 hours after transduction, TILs were transferred into a 6M GREX well plate (Wilson Wolf) in a total of 16-40 mL TIL media (RPMI-1640 supplemented with GlutaMAX (Thermo Fisher), 1% Penicillin/Streptomycin, 1 mM Sodium Pyruvate, 1% HEPES, 50 μM 2-Mercaptoethanol (Invitrogen) and 10% heat-inactivated human AB serum (Valley Bio)). Irradiated or Mitomycin-C treated K562 feeder cells transduced with 41BBL and mbIL21 as described in Example 2 were added to the culture at a ratio of 2:1 or 5:1 K562 to TIL. TILs transduced with the regulated mbIL15 construct received 25 μM Acetazolamide (SelleckChem) and untransduced TILs received 6000 IU/mL IL2. The cells were grown for 14 days in the GREX plates for the “rapid expansion protocol” or REP, and media was added or replaced as necessary. During the expansion, each GREX well was resuspended and mixed thoroughly, and an aliquot was taken for cell counting (Cellaca Cell Counter, Nexcelom) and flow cytometry staining. Samples were stained using antibodies CD3-BUV395 (BD), CD56-BV711 (Biolegend), CD4-BV605 (Biolegend), CD8-Alexa Fluor 700 (Biolegend), IL15-DyL650 (LakePharma, conjugated in-house), IL15RaFc-Biotin (ACRO Biosystems) with secondary Streptavidin-BV421 (Biolegend), and fixable viability dye eFluor 780 (Thermo Fisher). Samples were run on the BD Fortessa flow cytometer and analysis conducted using Flow Jo V10.7.1. The transduction efficiency was determined by percent of cells staining double positive for IL15-DyL650 and IL15RaFc-Biotin within the population of live, CD3 positive cells (FIG. 2).

TILs transduced with lentivirus comprising nucleic acid sequences encoding mbIL15 as described herein may be referred to in subsequent examples as “mbIL15 TILs.” TILs transduced with lentivirus comprising nucleic acid sequences encoding regulated mbIL15, such as OT-IL15-293, may also be referred to in subsequent examples as “regulated mbIL15 TILs.” TILs transduced with lentivirus comprising nucleic acid sequences encoding constitutive mbIL15, such as OT-IL15-292, may also be referred to in subsequent examples as “constitutive mbIL15 TILs.”

### Example 4. TIL Expansion in Rapid Expansion Protocol

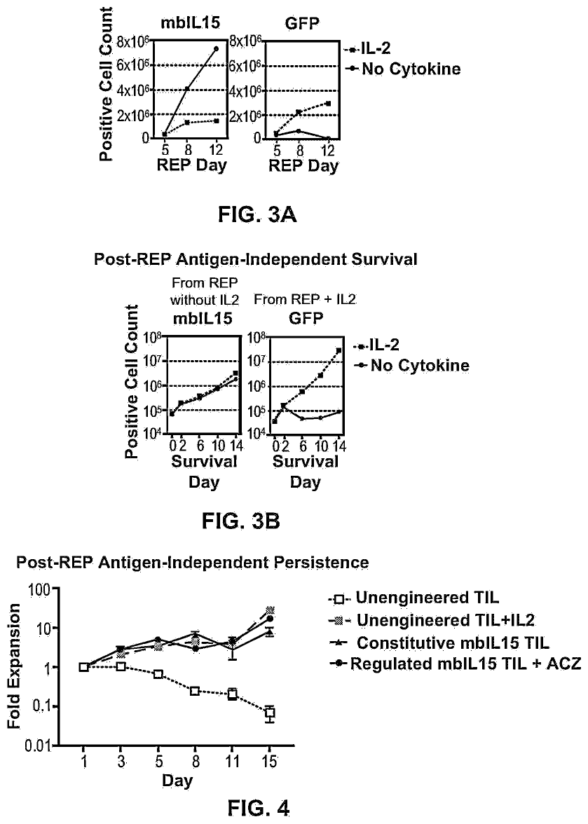

TILs and feeder cells were generated as described in Examples 1-3 above. Briefly, after 3 weeks in the pre-REP culture, cryopreserved TILs were thawed and rested overnight with 6000 IU/mL human IL2. TILs were then activated with anti-CD3/CD28 Dynabeads or on OKT3-coated multi-well plates for 24 hours, after which point they were transduced with constitutive mbIL15 (OT-IL15-292) or GFP (OT-EGFP-001) lentiviral vectors or unmodified as an unengineered condition. 24 hours after transduction, TILs were expanded with K562-IL21-41BBL feeder cells (2:1 ratio of feeder cells: TILs) in GREX 6M well plates (Wilson Wolf) with 6000 IU/mL I-2 added to unengineered TILs as well as experimental “+IL2” conditions. The cells were grown for 12 days in the GREX plates for the “rapid expansion protocol” or REP, and media was added or replaced as necessary. On days 5, 8, and 12 post-transduction, each GREX well was resuspended. An aliquot was taken for flow cytometry staining to quantify the number of IL15+ or GFP+ cells as described in Example 3 using antibodies CD3-BUV395 (BD), CD56-BV711 (Biolegend), CD4-BV605 (Biolegend), CD8-Alexa Fluor 700 (Biolegend), IL15-DyL650 (LakePharma, conjugated in-house), IL15RaFc-Biotin (ACRO Biosystems) with secondary Streptavidin-BV421 (Biolegend), and fixable viability dye eFluor 780 (Thermo Fisher). GFP-expressing TILs require exogenous IL2 for expansion in REP, while constitutive mb-IL15-expressing TILs expand in the absence of IL2 (FIG. 3A).

### Example 5. Expansion and Survival in an Antigen-Independent Setting

Next, post-REP TILs for assessed for their ability to persist or expand in the context of an in vitro antigen-independent survival assay. After 12 days of REP expansion, mbIL15 transduced cells that were expanded with no cytokine and GFP cells that were expanded with 6000 IU/mL IL2 were de-beaded, washed, and rested overnight with no cytokine. The next day, TILs were plated in a 48-well plate at 5×10⁵ cells/well in TIL media with or without added IL2 (6000 IU/mL, Peprotech). Cells were split or media was added every two days for a total duration of 10 days. An aliquot was also taken for flow cytometry staining every two days and the number of IL15+ or GFP+ cells was quantified as described in Example 3. Constitutive mbIL15 TILs expanded during the 14-day survival assay either with or without exogenous IL2, while GFP TILs required IL2 for expansion (FIG. 3B).

In a new study that included regulated mbIL15 expressing TILs, TILs and feeder cells were generated as described in Examples 1-3 above. Briefly, after 3 weeks in the pre-REP culture, cryopreserved TILs were thawed and rested overnight with 6000 IU/mL human IL2. TILs were then activated with anti-CD3/CD28 Dynabeads or on OKT3-coated multi-well plates for 24 hours, after which point they were transduced with constitutive mbIL15 (OT-IL15-292) or regulated mbIL15 (OT-IL15-293) lentiviral vectors or unengineered. 24 hours after transduction, TILs were expanded with K562-IL21-41BBL feeder cells (5:1 ratio of feeder cells: TILs) in GREX 6M well plates (Wilson Wolf) with 6000 IU/mL IL2 added to UT TILs, and 25 μM acetazolamide (SelleckChem) added to regulated mbIL15 TILs. After 14 days of expansion, TILs were isolated and plated in a multi-well plate at 5×10⁵ cells/well in TIL media with or without added IL2 (200 IU/mL, Peprotech) or acetazolamide (25 μM, SelleckChem). Entire wells were harvested for analysis of cell expansion by cell count (Celleca Cell Counter, Nexelom) and phenotype by flow cytometry (BD Fortessa) and fresh cytokine/ligand was added every 3 days. As demonstrated in FIG. 4, over the course of the 15 days of this assay, unengineered TILs did not expand without any exogenous cytokines (0.07±0.03-fold expansion), but with exogenous IL2 (200 IU/mL) were able to expand greater than twenty-fold (27.8±0.25-fold expansion). In contrast, modified TILs expanded significantly without the addition of any exogenous cytokines; after 15 days constitutive mbIL15 TILs expanded eight-fold (8.28±1.9-fold expansion), and regulated mbIL15 TILs given 25 μM acetazolamide expanded seventeen-fold (17.3±0.82-fold expansion). Without the addition of acetazolamide, regulated mbIL15 TILs expanded four-fold lower than with ligand (4.52±0.48-fold expansion), highlighting the role of acetazolamide in regulating survival of regulated mbIL15 TILs.

### Example 6. Expansion and Survival in an Antigen-Dependent Setting

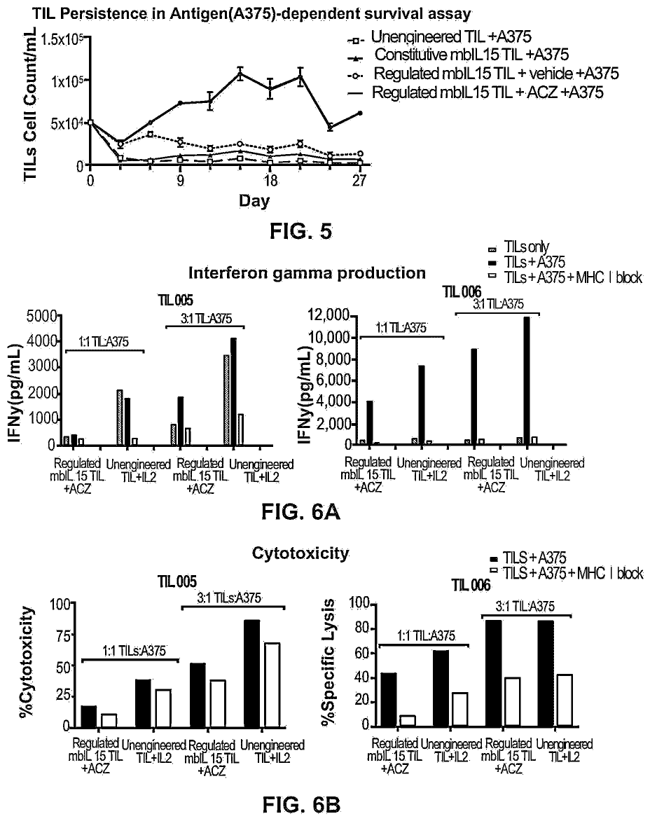

TILs and feeder cells were generated as described in Examples 1-3 above. Briefly, after 3 weeks in the pre-REP culture, cryopreserved TILs were thawed and rested overnight with 6000 IU/mL human IL2. TILs were then activated with anti-CD3/CD28 Dynabeads or on OKT3-coated multi-well plates for 24 hours, after which point they were transduced with regulated mbIL15 (OT-IL15-293) lentiviral vectors or unengineered. Twenty-four hours after transduction, TILs were expanded with K562-IL21-41BBL feeder cells (5:1 ratio of feeder cells: TILs) in GREX 6M well plates (Wilson Wolf) with 6000 IU/mL IL2 added to UT TILs, and 25 μM acetazolamide (SelleckChem) added to regulated mbIL15 TILs. After 14 days of expansion, TILs were cryopreserved in Bambanker freezing medium (Bulldog Bio). At a later time, cryopreserved TILs were thawed and rested overnight in TIL media with 200 IU/mL IL2 (unengineered TILs) or TIL media with 25 μM acetazolamide (regulated mbIL15 TILs). Following overnight rest, TILs were plated in a multi-well plate at 1:1 ratio with mitomycin C-treated melanoma cells in a TIL:tumor co-culture assay in TIL media with or without added IL2 (200 IU/mL, Peprotech) or acetazolamide (25 μM, SelleckChem), and the assay was sustained for 27 total days. A vehicle-only control was included for acetazolamide, with the identical volume of DMSO added to vehicle control groups. Melanoma cells were from the A375 cell line (ATCC), which was modified with a puromycin-dependent luciferase vector, and were treated with 10 μg/mL mitomycin C as described above (Example 3) to prevent proliferation of these tumor cells. Every 3 days, wells of this co-culture assay were mixed and an aliquot was isolated for analysis of cell expansion by cell count (Celleca Cell Counter, Nexelom) and phenotype by flow cytometry (BD Fortessa). Fresh mitomycin C-treated A375 melanoma cells as well as fresh cytokine/ligand in TIL media was added every 3 days. As demonstrated in FIG. 5, regulated mbIL15 TILs regulated with acetazolamide establish stable expansion kinetics, and even in this antigen-dependent setting, where the chronic stimulation should rapidly exhaust TILs and decrease cell counts, transduced TILs persisted. Over the assay, unengineered TILs did not expand without any exogenous cytokines (0.46±0.02-fold expansion from day 1 to day 27), but with exogenous IL2 (200 IU/mL) were able to expand greater than twenty five-fold (25.4=4.06-fold expansion from day 1 to day 27). In contrast, modified TILs expanded without the addition of any exogenous cytokines and notably regulated mbIL15 TILs given 25 μM acetazolamide expanded twelve-fold (12.2±0.10-fold expansion from day 1 to day 27). Without the addition of acetazolamide (with vehicle control only), regulated mbIL15 TILs expanded four-fold lower than with ligand (2.68-0.42-fold expansion from day 1 to day 27), highlighting the role of acetazolamide in regulating survival of regulated mbIL15 TILs.

### Example 7. Tumor Reactivity of Fresh Post-REP TILs

TILs from two melanoma donors were generated as described in Examples 1-3. Briefly, after 3 weeks in the pre-REP culture, cryopreserved TILs were thawed and rested overnight with 6000 IU/mL human IL2. TILs were then activated with anti-CD3/CD28 Dynabeads or on OKT3-coated multi-well plates for 24 hours, after which point they were transduced with regulated mbIL15 (OT-IL15-293) lentiviral vectors or unengineered. 24 hours after transduction, TILs were expanded with K562-IL21-41BBL feeder cells (5:1 ratio of feeder cells:TILs) in GREX 6M well plates (Wilson Wolf) with 6000 IU/mL IL2 added to unengineered TILs, and 25 μM acetazolamide (SelleckChem) added to regulated mbIL15 TILs. After 14 days of expansion, TILs were harvested, de-beaded, and rested overnight with and without IL2 and acetazolamide. Melanoma cell line expressing luciferase, A375-FLuc-Puro (ATCC) was resuspended in TIL media at 5×10⁶ cells/mL. 10 μg/mL Mitomycin-C was added to the cells, which were then incubated for 30 minutes at 37° C. The cells were then washed three times with 50 mL TIL media. 1×10⁵ A375 cells per well were added to a 96-well flat bottom tissue-culture treated plate. In some wells, 80 μg/mL HLA-ABC (Biolegend) blocking antibody was added to block MHC class I on the target cells. TILs that were rested overnight were added at a 1:1 or 3:1 ratio of TIL:A375 for a total volume of 200 μL per well. At a 48-hour time point, supernatant was saved from each well and the concentration of IFNγ was assayed by MSD. Lysis of the tumor cells was analyzed using CellTiterGlo Luminescent Cell Viability Assay (Promega), following manufacturer's protocol. Percent lysis was calculated as luminescence in the co-culture well minus background fluorescence divided by luminescence in A375-only control wells minus background fluorescence. Both untransduced TILs cultured with IL2 and regulated mbIL15 TILs expanded in REP in the absence of IL2 produce increased IFNγ in co-culture with the A375 melanoma line compared to TILs alone (FIG. 6A). Additionally, there was specific lysis of the tumor cells in co-culture conditions measured by decreased luminescence of the target cell line (FIG. 6B). Both percent specific lysis and IFNγ production was decreased in co-culture conditions with MHC class I blocking antibody, indicating that the cytotoxicity of the TILs against this tumor cell line is MHC class I dependent. This result is repeated in two melanoma donors.

### Example 8. mbIL15 TILs Persist Long-Term In Vivo without IL2

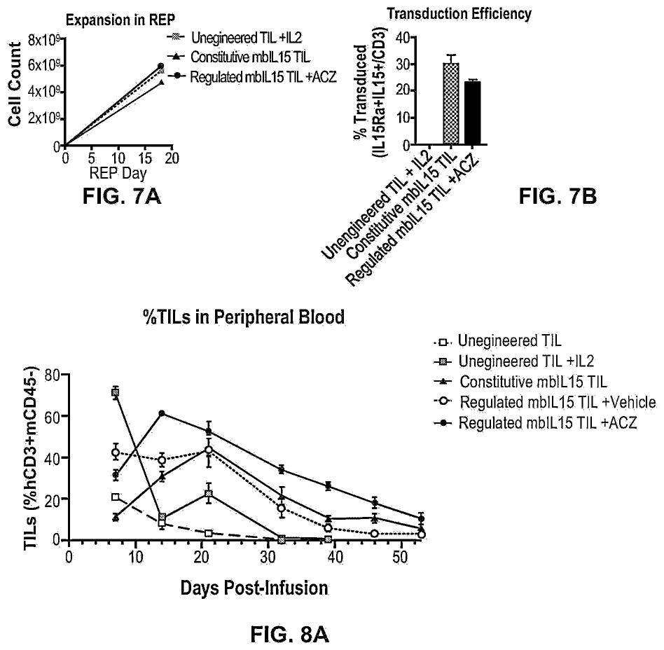

TILs from one donor and feeder cells were generated as described in Examples 1-3 above. Briefly, after 3 weeks in the pre-REP culture, cryopreserved TILs were thawed and rested overnight with 6000 IU/mL human IL2. TILs were then activated with anti-CD3/CD28 Dynabeads for 24 hours, after which point they were transduced with constitutive mbIL15 (OT-IL15-292) or regulated mbIL15 (OT-IL15-293) lentiviral vectors or unengineered. 24 hours after transduction, TILs were expanded with K562-IL21-41BBL feeder cells (5:1 ratio of feeder cells:TILs) in GREX 6M well plates (Wilson Wolf) with 6000 IU/mL IL2 added to unengineered TILs, and 25 μM acetazolamide (SelleckChem) added to regulated mbIL15 TILs. After 14 days of expansion, TILs were harvested, de-beaded, and prepared for adoptive cell transfer. Unengineered TILs expanded 612-fold, constitutive mbIL5 TILs expanded 1080-fold, and regulated mbIL15 TILs expanded 450-fold (FIG. 7A).

NSG (NOD.Cg-PrkdcscidIl2rgtm1Wjl/SzJ) mice were purchased from Jackson Laboratories. Six- to eight-week-old female mice were systemically infused with 10×10⁶ TILs/mouse, with or without exogenous IL2 (Proleukin), or clinical grade acetazolamide or vehicle, as described in Table 4.

TABLE 4
In Vivo Group Dosing

| Experimental | | | |
|---|---|---|---|
| Group | Cells | Exogenous Treatment | N |
| Unengineered | 10 × 10⁶ Untransduced | | 10 |
| | TILs/mouse | | |
| Unengineered + | 10 × 10⁶ Untransduced | 6 × 10⁵ IU | 10 |
| IL2 | TILs/mouse | Proleukin/mouse, | |
| | | dosed IP QD for the | |
| | | first 4 days on study | |
| Constitutive | 10 × 10⁶ constitutive | | 10 |
| mbIL15 | mbIL 15 transduced | | |
| | TILs/mouse | | |
| Regulated | 10 × 10⁶ regulated | 200 mg/kg PO | 10 |
| mbIL15 + ACZ | mbIL15 transduced | QD acetazolamide, | |
| | TILs/mouse | dosed daily | |
| Regulated | 10 × 10⁶ regulated | 200 mg/kg PO QD | 10 |
| mbIL15 + vehicle | mbIL15 transduced | vehicle, dosed daily | |
| | TILs/mouse | | |

TILs were assessed for IL15 expression on the day of adoptive cell therapy, and constitutive mbIL15 transduced TILs exhibited slightly higher levels of mbIL15 transduction (30.2±0.46% IL15+IL15RaFc+) than regulated mbIL15 transduced TILs (23.6±1.1% IL15+IL15RaFc+), but both transduced populations were acceptable for adoptive cell transfer (FIG. 7B). IL15 expression or transduction efficiency was assessed by flow cytometry; cells were incubated with Fc Block, and stained first with IL15 conjugated to DyL650 (Lake Pharma, conjugated in-house) and biotinylated IL15RaFc (ACROBiosystems). After incubating in the dark at room temperature for 25 minutes, cells were washed in FACS buffer, centrifuged, and resuspended in FACS buffer containing streptavidin conjugated to BV421 (Biolegend). After incubating in the dark at 4° C. for 20 minutes, cells were washed in FACS buffer, centrifuged, resuspended in FACS buffer, and samples were run on BD Fortessa flow cytometer. Analysis occurred with FlowJo V10.7.1.

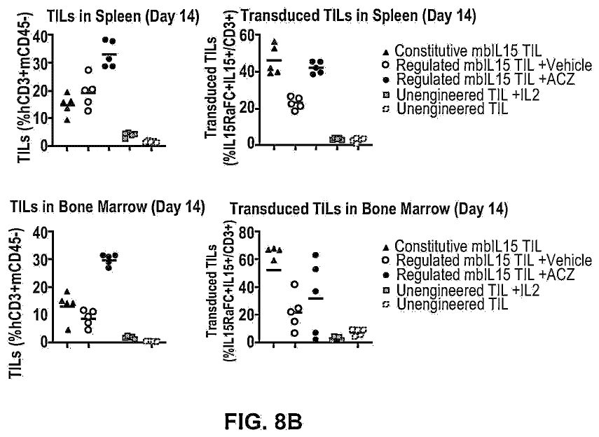

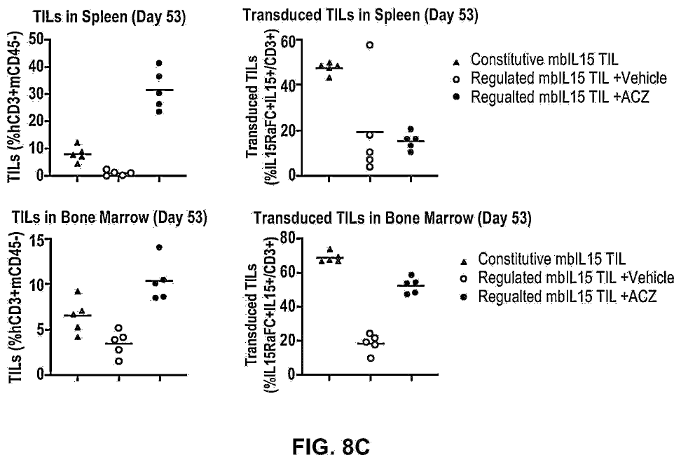

On days 7, 14, 21, 32, 39, 46, and 53 following adoptive cell therapy, 75 μL of systemic blood was isolated via submandibular vein collection in EDTA-containing tubes and processed for enumeration of TILs. Blood samples received 1-3 mL of ACK lysis buffer (Gibco) and were incubated for 10-20 minutes to lyse red blood cells (RBCs). After RBC lysis, samples were filtered through a 70 μm cell strainer, centrifuged, and resuspend in FACS buffer. An aliquot of each sample was isolated for analysis of cell count (Celleca Cell Counter, Nexelom) and the remainder was used for phenotype assessment by flow cytometry (BD Fortessa). For phenotypic assessment, blood samples were stained with antibodies specific for CD3 (BD), mouse CD45, CD25 (BD), FoxP3, CD4, CD8, IL15 (Lake Pharma, conjugated in-house), KLRG1, CD127, CD45RA, CD45RO, CD95, CD69, CCR7, CD56, and biotinylated IL15RaFc (ACROBiosystems). Antibodies were conjugated to FITC, PE, PE-Cy5, PE-Cy7, PerCP-Cy5.5, DyL650, APC-Cy7, BUV395, BUV737, BV421, BV510, BV605, BV711, or BV786 (Anti-human antibodies, all Biolegend, unless otherwise identified). In addition, a viability dye (e780 fixable viability dye, Invitrogen) was included for all samples. Samples were run on the BD Fortessa flow cytometer and analysis conducted using Flow Jo V10.7.1. To enumerate TILs throughout the study, TILs were gated as live cells, followed by lymphocytes, followed by human CD3+ and mouse CD45− cells. As seen in FIG. 8A, unengineered TILs rapidly declined in vivo, reaching undetectable levels by day 53 post-infusion. Unengineered TILs receiving exogenous IL2 fared better, although persistence was low by day 53 post-infusion, where quantified TILs were at 0.64±0.17%. In contrast, by day 53-post infusion it was clear that transduced TILs remained at detectable levels systemically, with constitutive mbI15 TILs at 5.73=1.2%. And regulated mbIL15 TILs+ACZ at 10.2±2.0%. The in vivo regulation effect of acetazolamide was clear, as by day 53 post-infusion regulated mbIL15 TILs+vehicle were nearly undetectable, at 2.94±0.36%.

On days 14 and 53 following adoptive cell therapy, a cohort of 5 animals per experimental group were sacrificed for terminal collection. From these animals, 200 μL of systemic blood was collected via cardiac puncture, the spleen was isolated, as well as bone marrow extracted from 1 femur. The blood was processed as described above. Spleens were mechanically disrupted through a 70 μm cell strainer, received ACK lysis for 3 minutes to lyse RBC, and were collected through a 70 μm cell strainer again. Bone marrow (BM) was flushed through one femur and collected through a 70 μm cell strainer. An aliquot of each processed tissue suspension was isolated for analysis of cell count (Celleca Cell Counter, Nexelom) and the remainder was used for phenotype assessment by flow cytometry (BD Fortessa). For phenotypic assessment, samples were stained with antibodies specific for CD3 (BD), mouse CD45, CD25 (BD), FoxP3, CD4, CD8, IL15 (Lake Pharma), KLRG1, CD127, CD45RA, CD45RO, CD95, CD69, CCR7, CD56, and biotinylated IL15RaFc (ACROBiosystems). Antibodies were conjugated to FITC, PE, PE-Cy5, PE-Cy7, PerCP-Cy5.5, DyL650, APC-Cy7, BUV395, BUV737, BV421, BV510, BV605, BV711, or BV786 (Anti-human antibodies, all Biolegend, unless otherwise identified). In addition, a viability dye (e780 fixable viability dye, Invitrogen) was included for all samples. Samples were run on the BD Fortessa flow cytometer and analysis conducted using Flow Jo V10.7.1. To enumerate TILs throughout the study, TILs were gated as live cells, followed by lymphocytes, followed by human CD3+ and mouse CD45-cells. As demonstrated in FIG. 8B and FIG. 8C, transduced TILs were identified at high levels in periphery lymphoid organs on day 14 as well as day 53 post-infusion, and ACZ-treated regulated mbIL15 TILs demonstrated significantly higher persistence than their vehicle-treated counterparts (p<0.005).

Table 5 shows viral vector sequences for the various constructs described herein.

TABLE 5
Viral vector sequences

| Viral | |
|---|---|
| Vector | NA Sequence |
| OT- | SEQ ID NO: 52 |
| EGFP-001 | gcgcgctcactggccgtcgttttacaacgtcgtgactgggaaaaccctggcgttacccaacttaatcgccttgcagcacatccccc |
| | tttcgccagctggcgtaatagcgaagaggcccgcaccgatcgcccttcccaacagttgcgcagcctgaatggcgaatgggacg |
| | cgccctgtagcggcgcattaagcgcggcgggtgtggtggttacgcgcagcgtgaccgctacacttgccagcgccctagcgccc |
| | gctcctttcgctttcttcccttcctttctcgccacgttcgccggctttccccgtcaagctctaaatcgggggctccctttagggttccga |
| | tttagtgctttacggcacctcgaccccaaaaaacttgattagggtgatggttcacgtagtgggccatcgccctgatagacggtttttc |
| | gccctttgacgttggagtccacgttctttaatagtggactcttgttccaaactggaacaacactcaaccctatctcggtctattcttttga |
| | tttataagggattttgccgatttcggcctattggttaaaaaatgagctgatttaacaaaaatttaacgcgaattttaacaaaatattaacg |
| | cttacaatttaggtggcacttttcggggaaatgtgcgcggaacccctatttgtttatttttctaaatacattcaaatatgtatccgctcatg |
| | agacaataaccctgataaatgcttcaataatattgaaaaaggaagagtatgagtattcaacatttccgtgtcgcccttattcccttttttg |
| | cggcattttgccttcctgtttttgctcacccagaaacgctggtgaaagtaaaagatgctgaagatcagttgggtgcacgagtgggtt |
| | acatcgaactggatctcaacagcggtaagatccttgagagttttcgccccgaagaacgttttccaatgatgagcacttttaaagttct |
| | gctatgtggcgcggtattatcccgtattgacgccgggcaagagcaactcggtcgccgcatacactattctcagaatgacttggttg |
| | agtactcaccagtcacagaaaagcatcttacggatggcatgacagtaagagaattatgcagtgctgccataaccatgagtgataa |
| | cactgcggccaacttacttctgacaacgatcggaggaccgaaggagctaaccgcttttttgcacaacatgggggatcatgtaactc |
| | gccttgatcgttgggaaccggagctgaatgaagccataccaaacgacgagcgtgacaccacgatgcctgtagcaatggcaaca |
| | acgttgcgcaaactattaactggcgaactacttactctagcttcccggcaacaattaatagactggatggaggcggataaagttgc |
| | aggaccacttctgcgctcggcccttccggctggctggtttattgctgataaatctggagccggtgagcgtgggtctcgcggtatcat |
| | tgcagcactggggccagatggtaagccccccgtatcgtagttatctacacgacggggagtcaggcaactatggatgaacgaaa |
| | tagacagatcgctgagataggtgcctcactgattaagcattggtaactgtcagaccaagtttactcatatatactttagattgatttaaa |
| | acttcatttttaatttaaaaggatctaggtgaagatcctttttgataatctcatgaccaaaatcccttaacgtgagttttcgttccactgag |
| | cgtcagaccccgtagaaaagatcaaaggatcttcttgagatcctttttttctgcgcgtaatctgctgcttgcaaacaaaaaaaccacc |
| | gctaccagcggtggtttgtttgccggatcaagagctaccaactctttttccgaaggtaactggcttcagcagagcgcagataccaa |
| | atactgttcttctagtgtagccgtagttaggccaccacttcaagaactctgtagcaccgcctacatacctcgctctgctaatcctgtta |
| | ccagtggctgctgccagtggcgataagtcgtgtcttaccgggttggactcaagacgatagttaccggataaggcgcagcggtcg |
| | ggctgaacggggggttcgtgcacacagcccagcttggagcgaacgacctacaccgaactgagatacctacagcgtgagctatg |
| | agaaagcgccacgcttcccgaagggagaaaggcggacaggtatccggtaagcggcagggtcggaacaggagagcgcacg |
| | agggagcttccagggggaaacgcctggtatctttatagtcctgtcgggtttcgccacctctgacttgagcgtcgatttttgtgatgct |
| | cgtcaggggggcggagcctatggaaaaacgccagcaacgcggcctttttacggttcctggccttttgctggccttttgctcacatg |
| | ttctttcctgcgttatcccctgattctgtggataaccgtattaccgcctttgagtgagctgataccgctcgccgcagccgaacgaccg |
| | agcgcagcgagtcagtgagcgaggaagcggaagagcgcccaatacgcaaaccgcctctccccgcgcgttggccgattcatta |
| | atgcagctggcacgacaggtttcccgactggaaagcgggcagtgagcgcaacgcaattaatgtgagttagctcactcattaggc |
| | accccaggctttacactttatgcttccggctcgtatgttgtgtggaattgtgagcggataacaatttcacacaggaaacagctatgac |
| | catgattacgccaagcgcgcaattaaccctcactaaagggaacaaaagctggagctgcaagcttaatgtagtcttatgcaatactc |
| | ttgtagtcttgcaacatggtaacgatgagttagcaacatgccttacaaggagagaaaaagcaccgtgcatgccgattggtggaagt |
| | aaggtggtacgatcgtgccttattaggaaggcaacagacgggtctgacatggattggacgaaccactgaattgccgcattgcaga |
| | gatattgtatttaagtgcctagctcgatacataaacgggtctctctggttagaccagatctgagcctgggagctctctggctaactag |
| | ggaacccactgcttaagcctcaataaagcttgccttgagtgcttcaagtagtgtgtgcccgtctgttgtgtgactctggtaactagag |
| | atccctcagacccttttagtcagtgtggaaaatctctagcagtggcgcccgaacagggacttgaaagcgaaagggaaaccagag |
| | gagctctctcgacgcaggactcggcttgctgaagcgcgcacggcaagaggcgaggggcggcgactggtgagtacgccaaaa |
| | attttgactagcggaggctagaaggagagagatgggtgcgagagcgtcagtattaagcgggggagaattagatcgcgatggga |
| | aaaaattcggttaaggccagggggaaagaaaaaatataaattaaaacatatagtatgggcaagcagggagctagaacgattcgc |
| | agttaatcctggcctgttagaaacatcagaaggctgtagacaaatactgggacagctacaaccatcccttcagacaggatcagaa |
| | gaacttagatcattatataatacagtagcaaccctctattgtgtgcatcaaaggatagagataaaagacaccaaggaagctttagac |
| | aagatagaggaagagcaaaacaaaagtaagaccaccgcacagcaagcggccgctgatcttcagacctggaggaggagatat |
| | gagggacaattggagaagtgaattatataaatataaagtagtaaaaattgaaccattaggagtagcacccaccaaggcaaagaga |
| | agagtggtgcagagagaaaaaagagcagtgggaataggagctttgttccttgggttcttgggagcagcaggaagcactatggg |
| | cgcagcgtcaatgacgctgacggtacaggccagacaattattgtctggtatagtgcagcagcagaacaatttgctgagggctatt |
| | gaggcgcaacagcatctgttgcaactcacagtctggggcatcaagcagctccaggcaagaatcctggctgtggaaagataccta |
| | aaggatcaacagctcctggggatttggggttgctctggaaaactcatttgcaccactgctgtgccttggaatgctagttggagtaat |
| | aaatctctggaacagatttggaatcacacgacctggatggagtgggacagagaaattaacaattacacaagcttaatacactcctt |
| | aattgaagaatcgcaaaaccagcaagaaaagaatgaacaagaattattggaattagataaatgggcaagtttgtggaattggttta |
| | acataacaaattggctgtggtatataaaattattcataatgatagtaggaggcttggtaggtttaagaatagtttttgctgtactttctata |
| | gtgaatagagttaggcagggatattcaccattatcgtttcagacccacctcccaaccccgaggggacccgacaggcccgaagga |
| | atagaagaagaaggtggagagagagacagagacagatccattcgattagtgaacggatctcgacggtatcgattagactgtagc |
| | ccaggaatatggcagctagattgtacacatttagaaggaaaagttatcttggtagcagttcatgtagccagtggatatatagaagca |
| | gaagtaattccagcagagacagggcaagaaacagcatacttcctcttaaaattagcaggaagatggccagtaaaaacagtacat |
| | acagacaatggcagcaatttcaccagtactacagttaaggccgcctgttggtgggcggggatcaagcaggaatttggcattccct |
| | acaatccccaaagtcaaggagtaatagaatctatgaataaagaattaaagaaaattataggacaggtaagagatcaggctgaaca |
| | tcttaagacagcagtacaaatggcagtattcatccacaattttaaaagaaaaggggggattggggggtacagtgcaggggaaag |
| | aatagtagacataatagcaacagacatacaaactaaagaattacaaaaacaaattacaaaaattcaaaattttcgggtttattacagg |
| | gacagcagagatccagtttggctgcattgatcacgtgaggctccggtgcccgtcagtgggcagagcgcacatcgcccacagtc |
| | cccgagaagttggggggaggggtcggcaattgaaccggtgcctagagaaggtggcgcggggtaaactgggaaagtgatgtc |
| | gtgtactggctccgcctttttcccgagggtgggggagaaccgtatataagtgcagtagtcgccgtgaacgttctttttcgcaacggg |
| | tttgccgccagaacacaggtaagtgccgtgtgtggttcccgcgggcctggcctctttacgggttatggcccttgcgtgccttgaatt |
| | acttccacctggctgcagtacgtgattcttgatcccgagcttcgggttggaagtggggggagagttcgaggccttgcgcttaagg |
| | agccccttcgcctcgtgcttgagttgaggcctggcctgggcgctggggccgccgcgtgcgaatctggtggcaccttcgcgcctg |
| | tctcgctgctttcgataagtctctagccatttaaaatttttgatgacctgctgcgacgctttttttctggcaagatagtcttgtaaatgcgg |
| | gccaagatctgcacactggtatttcggtttttggggccgcgggcggcgacggggcccgtgcgtcccagcgcacatgttcggcga |
| | ggggggcctgcgagcgcggccaccgagaatcggacgggggtagtctcaagctggccggcctgctctggtgcctggcctcg |
| | cgccgccgtgtatcgccccgccctgggcggcaaggctggcccggtcggcaccagttgcgtgagcggaaagatggccgcttcc |
| | cggccctgctgcagggagctcaaaatggaggacgcggcgctcgggagagcggggggtgagtcacccacacaaaggaaaa |
| | gggcctttccgtcctcagccgtcgcttcatgtgactccactgagtaccgggcgccgtccaggcacctcgattagttctcgagctttt |
| | ggagtacgtcgtctttaggttggggggaggggttttatgcgatggagtttccccacactgagtgggtggagactgaagttaggcc |
| | agcttggcacttgatgtaattctccttggaatttgccctttttgagtttggatcttggttcattctcaagcctcagacagtggttcaaagtt |
| | tttttcttccatttcaggtgtcgtgatctagaggatccACCatggtgagcaagggcgaggagctgttcaccggggtggtgcccat |
| | cctggtcgagctggacggcgacgtaaacggccacaagttcagcgtgtccggcgagggcgagggcgatgccacctacggcaa |
| | gctgaccctgaagttcatctgcaccaccggcaagctgcccgtgccctggcccaccctcgtgaccaccctgacctacggcgtgca |
| | gtgcttcagccgctaccccgaccacatgaagcagcacgacttcttcaagtccgccatgcccgaaggctacgtccaggagcgca |
| | ccatcttcttcaaggacgacggcaactacaagacccgcgccgaggtgaagttcgagggcgacaccctggtgaaccgcatcgag |
| | ctgaagggcatcgacttcaaggaggacggcaacatcctggggcacaagctggagtacaactacaacagccacaacgtctatat |
| | catggccgacaagcagaagaacggcatcaaggtgaacttcaagatccgccacaacatcgaggacggcagcgtgcagctcgcc |
| | gaccactaccagcagaacacccccatcggcgacggccccgtgctgctgcccgacaaccactacctgagcacccagtccgccc |
| | tgagcaaagaccccaacgagaagcgcgatcacatggtcctgctggagttcgtgaccgccgccgggatcactctcggcatggac |
| | gagctgtacaagtaagtcgacaatcaacctctggattacaaaatttgtgaaagattgactggtattcttaactatgttgctccttttacg |
| | ctatgtggatacgctgctttaatgcctttgtatcatgctattgcttcccgtatggctttcattttctcctccttgtataaatcctggttgctgt |
| | ctctttatgaggagttgtggcccgttgtcaggcaacgtggcgtggtgtgcactgtgtttgctgacgcaacccccactggttggggc |
| | attgccaccacctgtcagctcctttccgggactttcgctttccccctccctattgccacggcggaactcatcgccgcctgccttgccc |
| | gctgctggacaggggctcggctgttgggcactgacaattccgtggtgttgtcggggaagctgacgtcctttccatggctgctcgc |
| | ctgtgttgccacctggattctgcgcgggacgtccttctgctacgtcccttcggccctcaatccagcggaccttccttcccgcggcct |
| | gctgccggctctgcggcctcttccgcgtcttcgccttcgccctcagacgagtcggatctccctttgggccgcctccccgcctggaa |
| | ttcgagctcggtacctttaagaccaatgacttacaaggcagctgtagatcttagccactttttaaaagaaaaggggggactggaag |
| | ggctaattcactcccaacgaagacaagatctgctttttgcttgtactgggtctctctggttagaccagatctgagcctgggagctctct |
| | ggctaactagggaacccactgcttaagcctcaataaagcttgccttgagtgcttcaagtagtgtgtgcccgtctgttgtgtgactctg |
| | gtaactagagatccctcagacccttttagtcagtgtggaaaatctctagcagtagtagttcatgtcatcttattattcagtatttataactt |
| | gcaaagaaatgaatatcagagagtgagaggaacttgtttattgcagcttataatggttacaaataaagcaatagcatcacaaatttca |
| | caaataaagcatttttttcactgcattctagttgtggtttgtccaaactcatcaatgtatcttatcatgtctggctctagctatcccgcccct |
| | aactccgcccatcccgcccctaactccgcccagttccgcccattctccgccccatggctgactaattttttttatttatgcagaggcc |
| | gaggccgcctcggcctctgagctattccagaagtagtgaggaggcttttttggaggcctagggacgtacccaattcgccctatagt |
| | gagtcgtattac |
| OT-IL15- | SEQ ID NO: 53 |
| 292 | tttgagtgagctgataccgctcgccgcagccgaacgaccgagcgcagcgagtcagtgagcgaggaagcggaagagcgccca |
| | atacgcaaaccgcctctccccgcgcgttggccgattcattaatgcagctggcacgacaggtttcccgactggaaagcgggcagt |
| | gagcgcaacgcaattaatgtgagttagctcactcattaggcaccccaggctttacactttatgcttccggctcgtatgttgtgtggaat |
| | tgtgagcggataacaatttcacacaggaaacagctatgaccatgattacgccaagcgcgcaattaaccctcactaaagggaacaa |
| | aagctggagctgcaagcttaatgtagtcttatgcaatactcttgtagtcttgcaacatggtaacgatgagttagcaacatgccttaca |
| | aggagagaaaaagcaccgtgcatgccgattggtggaagtaaggtggtacgatcgtgccttattaggaaggcaacagacgggtc |
| | tgacatggattggacgaaccactgaattgccgcattgcagagatattgtatttaagtgcctagctcgatacaataaacgggtctctct |
| | ggttagaccagatctgagcctgggagctctctggctaactagggaacccactgcttaagcctcaataaagcttgccttgagtgcttc |
| | aagtagtgtgtgcccgtctgttgtgtgactctggtaactagagatccctcagacccttttagtcagtgtggaaaatctctagcagtgg |
| | cgcccgaacagggacctgaaagcgaaagggaaaccagagctctctcgacgcaggactcggcttgctgaagcgcgcacggca |
| | agaggcgaggggcggcgactggtgagtacgccaaaaattttgactagcggaggctagaaggagagagatgggtgcgagagc |
| | gtcagtattaagcgggggagaattagatcgcgatgggaaaaaattcggttaaggccagggggaaagaaaaaatataaattaaaa |
| | catatagtatgggcaagcagggagctagaacgattcgcagttaatcctggcctgttagaaacatcagaaggctgtagacaaatac |
| | tgggacagctacaaccatcccttcagacaggatcagaagaacttagatcattatataatacagtagcaaccctctattgtgtgcatc |
| | aaaggatagagataaaagacaccaaggaagctttagacaagatagaggaagagcaaaacaaaagtaagaccaccgcacagc |
| | aagcggccgctgatcttcagacctggaggaggagatatgagggacaattggagaagtgaattatataaatataaagtagtaaaaa |
| | ttgaaccattaggagtagcacccaccaaggcaaagagaagagtggtgcagagagaaaaaagagcagtgggaataggagcttt |
| | gttccttgggttcttgggagcagcaggaagcactatgggcgcagcctcaatgacgctgacggtacaggccagacaattattgtct |
| | ggtatagtgcagcagcagaacaatttgctgagggctattgaggcgcaacagcatctgttgcaactcacagtctggggcatcaagc |
| | agctccaggcaagaatcctggctgtggaaagatacctaaaggatcaacagctcctggggatttggggttgctctggaaaactcatt |
| | tgcaccactgctgtgccttggaatgctagttggagtaataaatctctggaacagattggaatcacacgacctggatggagtgggac |
| | agagaaattaacaattacacaagcttaatacactccttaattgaagaatcgcaaaaccagcaagaaaagaatgaacaagaattatt |
| | ggaattagataaatgggcaagtttgtggaattggtttaacataacaaattggctgtggtatataaaattattcataatgatagtaggag |
| | gcttggtaggtttaagaatagtttttgctgtactttctatagtgaatagagttaggcagggatattcaccattatcgtttcagacccacct |
| | cccaaccccgaggggacccgacaggcccgaaggaatagaagaagaaggtggagagagagacagagacagatccattcgat |
| | tagtgaacggatctcgacggtatcgattagactgtagcccaggaatatggcagctagattgtacacatttagaaggaaaagttatct |
| | tggtagcagttcatgtagccagtggatatatagaagcagaagtaattccagcagagacagggcaagaaacagcatacttcctctta |
| | aaattagcaggaagatggccagtaaaaacagtacatacagacaatggcagcaatttcaccagtactacagttaaggccgcctgtt |
| | ggtgggcggggatcaagcaggaatttggcattccctacaatccccaaagtcaaggagtaatagaatctatgaataaagaattaaa |
| | gaaaattataggacaggtaagagatcaggctgaacatcttaagacagcagtacaaatggcagtattcatccacaattttaaaagaa |
| | aaggggggattggggggtacagtgcaggggaaagaatagtagacataatagcaacagacatacaaactaaagaattacaaaaa |
| | caaattacaaaaattcaaaattttcgggtttattacagggacagcagagatccagtttggctgcatacgcgtcgtgaggctccggtg |
| | cccgtcagtgggcagagcgcacatcgcccacagtccccgagaagttggggggaggggtcggcaattgaaccggtgcctaga |
| | gaaggtggcgcggggtaaactgggaaagtgatgtcgtgtactggctccgcctttttcccgaggggggggagaaccgtatataa |
| | gtgcagtagtcgccgtgaacgttctttttcgcaacgggtttgccgccagaacacaggtaagtgccgtgtgtggttcccgcgggcct |
| | ggcctctttacgggttatggcccttgcgtgccttgaattacttccacctggctgcagtacgtgattcttgatcccgagcttcgggttgg |
| | aagtgggtgggagagttcgaggccttgcgcttaaggagccccttcgcctcgtgcttgagttgaggcctggcctgggcgctgggg |
| | ccgccgcgtgcgaatctggtggcaccttcgcgcctgtctcgctgctttcgataagtctctagccatttaaaatttttgatgacctgctg |
| | cgacgctttttttctggcaagatagtcttgtaaatgcgggccaagatctgcacactggtatttcggtttttggggccgcgggcggcg |
| | acggggcccgtgcgtcccagcgcacatgttcggcgaggcggggcctgcgagcgcggccaccgagaatcggacgggggtag |
| | tctcaagctggccggcctgctctggtgcctggcctcgcgccgccgtgtatcgccccgccctgggcggcaaggctggcccggtc |
| | ggcaccagttgcgtgagcggaaagatggccgcttcccggccctgctgcagggagctcaaaatggaggacgcggcgctcggg |
| | agagcggggggtgagtcacccacacaaaggaaaagggcctttccgtcctcagccgtcgcttcatgtgactccacTgagtacc |
| | gggcgccgtccaggcacctcgattagttctcgTgcttttggagtacgtcgtctttaggttggggggaggggttttatgcgatggag |
| | tttccccacactgagtgggtggagactgaagttaggccagcttggcacttgatgtaattctccttggaatttgccctttttgagtttgga |
| | tcttggttcattctcaagcctcagacagtggttcaaagtttttttcttccatttcaggtgtcgtgagctagACTAGTaccATGG |
| | ACATGCGGGTGCCTGCACAACTTCTGGGCCTGCTGTTGTTGTGGCTGTCTGGA |
| | GCCCGGTGTAATTGGGTAAATGTTATCAGTGATCTCAAGAAGATAGAGGATCT |
| | CATCCAGTCCATGCATATTGATGCCACGCTGTACACAGAAAGCGATGTGCATC |
| | CTAGCTGTAAGGTGACAGCGATGAAGTGTTTTCTTTTGGAGCTGCAGGTAATT |
| | AGTCTTGAGTCCGGCGATGCCAGCATTCATGATACCGTAGAAAACTTGATTAT |
| | CCTGGCCAACAATTCTCTGTCCTCAAACGGAAACGTAACCGAGAGCGGTTGTA |
| | AAGAATGTGAAGAACTGGAAGAAAAGAACATCAAGGAGTTTCTGCAATCATT |
| | CGTTCACATCGTACAAATGTTCATAAATACGTCAGGATCTGGTTCTGGTTCCG |
| | GAAGTGGATCTGGTTCAGGGTCCGGTAGTGGATCTGGGTCAGGAAGTGGAAG |
| | CGGTAGTGGGTCTGGATCTAAACAAGAGCACTTTCCTGATAACCTGTTGCCGA |
| | GCTGGGCGATTACGCTTATCAGTGTAAACGGCATCTTTGTAATATGCTGTCTG |
| | ACCTACTGCTTCGCACCAAGGTGCCGGGAGAGAAGGAGAAATGAAAGACTGA |
| | GAAGGGAGAGCGTGAGACCTGTGGGATCCtaaGCTAGCGTCGGCaatcaacctctggatt |
| | acaaaatttgtgaaagattgactggtattcttaactatgttgctccttttacgctatgtggatacgctgctttaatgcctttgtatcatgcta |
| | ttgcttcccgtatggctttcattttctcctccttgtataaatcctggttgctgtctctttatgaggagttgtggcccgttgtcaggcaacgt |
| | ggcgtggtgtgcactgtgtttgctgacgcaacccccactggttggggcattgccaccacctgtcagctcctttccgggactttcgct |
| | ttccccctccctattgccacggcggaactcatcgccgcctgccttgcccgctgctggacaggggctcggctgttgggcactgaca |
| | attccgtggtgttgtcggggaagctgacgtcctttccatggctgctcgcctgtgttgccacctggattctgcgcgggacgtccttctg |
| | ctacgtcccttcggccctcaatccagcggaccttccttcccgcggcctgctgccggctctgcggcctcttccgcgtcttcgccttcg |
| | ccctcagacgagtcggatctccctttgggccgcctccccgcctggaattcgagctcggtacctttaagaccaatgacttacaaggc |
| | agctgtagatcttagccactttttaaaagaaaaggggggactggaagggctaattcactcccaacgaagacaagatctgctttttgc |
| | ttgtactgggtctctctggttagaccagatctgagcctgggagctctctggctaactagggaacccactgcttaagcctcaataaag |
| | cttgccttgagtgcttcaagtagtgtgtgcccgtctgttgtgtgactctggtaactagagatccctcagacccttttagtcagtgtgga |
| | aaatctctagcagtagtagttcatgtcatcttattattcagtatttataacttgcaaagaaatgaatatcagagagtgagaggaacttgtt |
| | tattgcagcttataatggttacaaataaagcaatagcatcacaaatttcacaaataaagcatttttttcactgcattctagttgtggtttgt |
| | ccaaactcatcaatgtatcttatcatgtctggctctagctatcccgcccctaactccgcccagttccgcccattctccgccccatggct |
| | gactaattttttttatttatgcagaggccgaggccgcctcggcctctgagctattccagaagtagtgaggaggcttttttggaggcct |
| | aggcttttgcgtcgagacgtacccaattcgccctatagtgagtcgtattacgcgcgctcactggccgtcgttttacaacgtcgtgact |
| | gggaaaaccctggcgttacccaacttaatcgccttgcagcacatccccctttcgccagctggcgtaatagcgaagaggcccgca |
| | ccgatcgcccttcccaacagttgcgcagcctgaatggcgaatggcgcgacgcgccctgtagcggcgcattaagcgcggcggg |
| | tgtggtggttacgcgcagcgtgaccgctacacttgccagcgccctagcgcccgctcctttcgctttcttcccttcctttctcgccacg |
| | ttcgccggctttccccgtcaagctctaaatcgggggctccctttagggttccgatttagtgctttacggcacctcgaccccaaaaaa |
| | cttgattagggtgatggttcacgtagtgggccatcgccctgatagacggtttttcgccctttgacgttggagtccacgttctttaatagt |
| | ggactcttgttccaaactggaacaacactcaaccctatctcggtctattcttttgatttataagggattttgccgatttcggcctattggtt |
| | aaaaaatgagctgatttaacaaaaatttaacgcgaattttaacaaaatattaacgtttacaatttcccaggtggcacttttcggggaaa |
| | tgtgcgcggaacccctatttgtttatttttctaaatacattcaaatatgtatccgctcatgagacaataaccctgataaatgcttcaataat |
| | attgaaaaaggaagagtatgagtattcaacatttccgtgtcgcccttattcccttttttgcggcattttgccttcctgtttttgctcaccca |
| | gaaacgctggtgaaagtaaaagatgctgaagatcagttgggtgcacgagtgggttacatcgaactggatctcaacagcggtaag |
| | atccttgagagttttcgccccgaagaacgttttccaatgatgagcacttttaaagttctgctatgtggcgcggtattatcccgtattgac |
| | gccgggcaagagcaactcggtcgccgcatacactattctcagaatgacttggttgagtactcaccagtcacagaaaagcatctta |
| | cggatggcatgacagtaagagaattatgcagtgctgccataaccatgagtgataacactgcggccaacttacttctgacaacgatc |
| | ggaggaccgaaggagctaaccgcttttttgcacaacatgggggatcatgtaactcgccttgatcgttgggaaccggagctgaatg |
| | aagccataccaaacgacgagcgtgacaccacgatgcctgtagcaatggcaacaacgttgcgcaaactattaactggcgaactac |
| | ttactctagcttcccggcaacaattaatagactggatggaggcggataaagttgcaggaccacttctgcgctcggcccttccggct |
| | ggctggtttattgctgataaatctggagccggtgagcgtgggtctcgcggtatcattgcagcactggggccagatggtaagccctc |
| | ccgtatcgtagttatctacacgacggggagtcaggcaactatggatgaacgaaatagacagatcgctgagataggtgcctcactg |
| | attaagcattggtaactgtcagaccaagtttactcatatatactttagattgatttaaaacttcatttttaatttaaaaggatctaggtgaa |
| | gatcctttttgataatctcatgaccaaaatcccttaacgtgagttttcgttccactgagcgtcagaccccgtagaaaagatcaaagga |
| | tcttcttgagatcctttttttctgcgcgtaatctgctgcttgcaaacaaaaaaaccaccgctaccagcggtggtttgtttgccggatcaa |
| | gagctaccaactctttttccgaaggtaactggcttcagcagagcgcagataccaaatactgtccttctagtgtagccgtagttaggc |
| | caccacttcaagaactctgtagcaccgcctacatacctcgctctgctaatcctgttaccagtggctgctgccagtggcgataagtcg |
| | tgtcttaccgggttggactcaagacgatagttaccggataaggcgcagcggtcgggctgaacggggggttcgtgcacacagcc |
| | cagcttggagcgaacgacctacaccgaactgagatacctacagcgtgagctatgagaaagcgccacgcttcccgaagggaga |
| | aaggcggacaggtatccggtaagcggcagggtcggaacaggagagcgcacgagggagcttccagggggaaacgcctggta |
| | tctttatagtcctgtcgggtttcgccacctctgacttgagcgtcgatttttgtgatgctcgtcaggggggcggagcctatggaaaaac |
| | gccagcaacgcggcctttttacggttcctggccttttgctggccttttgctcacatgttctttcctgcgttatcccctgattctgtggata |
| | accgtattaccgcc |
| OT-IL15- | SEQ ID NO: 54 |
| 293 | tttgagtgagctgataccgctcgccgcagccgaacgaccgagcgcagcgagtcagtgagcgaggaagcggaagagcgccca |
| | atacgcaaaccgcctctccccgcgcgttggccgattcattaatgcagctggcacgacaggtttcccgactggaaagcgggcagt |
| | gagcgcaacgcaattaatgtgagttagctcactcattaggcaccccaggctttacactttatgcttccggctcgtatgttgtgtggaat |
| | tgtgagcggataacaatttcacacaggaaacagctatgaccatgattacgccaagcgcgcaattaaccctcactaaagggaacaa |
| | aagctggagctgcaagcttaatgtagtcttatgcaatactcttgtagtcttgcaacatggtaacgatgagttagcaacatgccttaca |
| | aggagagaaaaagcaccgtgcatgccgattggtggaagtaaggtggtacgatcgtgccttattaggaaggcaacagacgggtc |
| | tgacatggattggacgaaccactgaattgccgcattgcagagatattgtatttaagtgcctagctcgatacaataaacgggtctctct |
| | ggttagaccagatctgagcctgggagctctctggctaactagggaacccactgcttaagcctcaataaagcttgccttgagtgcttc |
| | aagtagtgtgtgcccgtctgttgtgtgactctggtaactagagatccctcagacccttttagtcagtgtggaaaatctctagcagtgg |
| | cgcccgaacagggacctgaaagcgaaagggaaaccagagctctctcgacgcaggactcggcttgctgaagcgcgcacggca |
| | agaggcgaggggcggcgactggtgagtacgccaaaaattttgactagcggaggctagaaggagagagatgggtgcgagagc |
| | gtcagtattaagcgggggagaattagatcgcgatgggaaaaaattcggttaaggccagggggaaagaaaaaatataaattaaaa |
| | catatagtatgggcaagcagggagctagaacgattcgcagttaatcctggcctgttagaaacatcagaaggctgtagacaaatac |
| | tgggacagctacaaccatcccttcagacaggatcagaagaacttagatcattatataatacagtagcaaccctctattgtgtgcatc |
| | aaaggatagagataaaagacaccaaggaagctttagacaagatagaggaagagcaaaacaaaagtaagaccaccgcacagc |
| | aagcggccgctgatcttcagacctggaggaggagatatgagggacaattggagaagtgaattatataaatataaagtagtaaaaa |
| | ttgaaccattaggagtagcacccaccaaggcaaagagaagagtggtgcagagagaaaaaagagcagtgggaataggagcttt |
| | gttccttgggttcttgggagcagcaggaagcactatgggcgcagcctcaatgacgctgacggtacaggccagacaattattgtct |
| | ggtatagtgcagcagcagaacaatttgctgagggctattgaggcgcaacagcatctgttgcaactcacagtctggggcatcaagc |
| | agctccaggcaagaatcctggctgtggaaagatacctaaaggatcaacagctcctggggatttggggttgctctggaaaactcatt |
| | tgcaccactgctgtgccttggaatgctagttggagtaataaatctctggaacagattggaatcacacgacctggatggagtgggac |
| | agagaaattaacaattacacaagcttaatacactccttaattgaagaatcgcaaaaccagcaagaaaagaatgaacaagaattatt |
| | ggaattagataaatgggcaagtttgtggaattggtttaacataacaaattggctgtggtatataaaattattcataatgatagtaggag |
| | gcttggtaggtttaagaatagtttttgctgtactttctatagtgaatagagttaggcagggatattcaccattatcgtttcagacccacct |
| | cccaaccccgaggggacccgacaggcccgaaggaatagaagaagaaggtggagagagagacagagacagatccattcgat |
| | tagtgaacggatctcgacggtatcgattagactgtagcccaggaatatggcagctagattgtacacatttagaaggaaaagttatct |
| | tggtagcagttcatgtagccagtggatatatagaagcagaagtaattccagcagagacagggcaagaaacagcatacttcctctta |
| | aaattagcaggaagatggccagtaaaaacagtacatacagacaatggcagcaatttcaccagtactacagttaaggccgcctgtt |
| | ggtgggcggggatcaagcaggaatttggcattccctacaatccccaaagtcaaggagtaatagaatctatgaataaagaattaaa |
| | gaaaattataggacaggtaagagatcaggctgaacatcttaagacagcagtacaaatggcagtattcatccacaattttaaaagaa |
| | aaggggggattggggggtacagtgcaggggaaagaatagtagacataatagcaacagacatacaaactaaagaattacaaaaa |
| | caaattacaaaaattcaaaattttcgggtttattacagggacagcagagatccagtttggctgcatacgcgtcgtgaggctccggtg |
| | cccgtcagtgggcagagcgcacatcgcccacagtccccgagaagttggggggaggggtcggcaattgaaccggtgcctaga |
| | gaaggtggcgcggggtaaactgggaaagtgatgtcgtgtactggctccgcctttttcccgaggggggggagaaccgtatataa |
| | gtgcagtagtcgccgtgaacgttctttttcgcaacgggtttgccgccagaacacaggtaagtgccgtgtgtggttcccgcgggcct |
| | ggcctctttacgggttatggcccttgcgtgccttgaattacttccacctggctgcagtacgtgattcttgatcccgagcttcgggttgg |
| | aagtgggtgggagagttcgaggccttgcgcttaaggagccccttcgcctcgtgcttgagttgaggcctggcctgggcgctgggg |
| | ccgccgcgtgcgaatctggtggcaccttcgcgcctgtctcgctgctttcgataagtctctagccatttaaaatttttgatgacctgctg |
| | cgacgctttttttctggcaagatagtcttgtaaatgcgggccaagatctgcacactggtatttcggtttttggggccgcgggcggcg |
| | acggggcccgtgcgtcccagcgcacatgttcggcgaggggggcctgcgagcgcggccaccgagaatcggacgggggtag |
| | tctcaagctggccggcctgctctggtgcctggcctcgcgccgccgtgtatcgccccgccctgggcggcaaggctggcccggtc |
| | ggcaccagttgcgtgagcggaaagatggccgcttcccggccctgctgcagggagctcaaaatggaggacgcggcgctcggg |
| | agagcggggggtgagtcacccacacaaaggaaaagggcctttccgtcctcagccgtcgcttcatgtgactccacTgagtacc |
| | gggcgccgtccaggcacctcgattagttctcgTgcttttggagtacgtcgtctttaggttggggggaggggttttatgcgatggag |
| | tttccccacactgagtgggtggagactgaagttaggccagcttggcacttgatgtaattctccttggaatttgccctttttgagtttgga |
| | tcttggttcattctcaagcctcagacagtggttcaaagtttttttcttccatttcaggtgtcgtgagctagACTAGTaccATGG |
| | ACATGCGGGTGCCTGCACAACTTCTGGGCCTGCTGTTGTTGTGGCTGTCTGGA |
| | GCCCGGTGTAATTGGGTAAATGTTATCAGTGATCTCAAGAAGATAGAGGATCT |
| | CATCCAGTCCATGCATATTGATGCCACGCTGTACACAGAAAGCGATGTGCATC |
| | CTAGCTGTAAGGTGACAGCGATGAAGTGTTTTCTTTTGGAGCTGCAGGTAATT |
| | AGTCTTGAGTCCGGCGATGCCAGCATTCATGATACCGTAGAAAACTTGATTAT |
| | CCTGGCCAACAATTCTCTGTCCTCAAACGGAAACGTAACCGAGAGCGGTTGTA |
| | AAGAATGTGAAGAACTGGAAGAAAAGAACATCAAGGAGTTTCTGCAATCATT |
| | CGTTCACATCGTACAAATGTTCATAAATACGTCAGGATCTGGTTCTGGTTCCG |
| | GAAGTGGATCTGGTTCAGGGTCCGGTAGTGGATCTGGGTCAGGAAGTGGAAG |
| | CGGTAGTGGGTCTGGATCTAAACAAGAGCACTTTCCTGATAACCTGTTGCCGA |
| | GCTGGGCGATTACGCTTATCAGTGTAAACGGCATCTTTGTAATATGCTGTCTG |
| | ACCTACTGCTTCGCACCAAGGTGCCGGGAGAGAAGGAGAAATGAAAGACTGA |
| | GAAGGGAGAGCGTGAGACCTGTGGGATCCTCCCATCACTGGGGGTACGGCAA |
| | ACACAACGGACCTGAGCACTGGCATAAGGACTTCCCCATTGCCAAGGGAGAG |
| | CGCCAGTCCCCTGTTGACATCGACACTCATACAGCCAAGTATGACCCTTCCCT |
| | GAAGCCCCTGTCTGTTTCCTATGATCAAGCAACTTCCCTGAGAATCCTCAACA |
| | ATGGTCATGCTTTCAACGTGGAGTTTGATGACTCTCAGGACAAAGCAGTGCTC |
| | AAGGGAGGACCCCTGGATGGCACTTACAGATTGATTCAGTTTCACTTTCACTG |
| | GGGTTCACTTGATGGACAAGGTTCAGAGCATACTGTGGATAAAAAGAAATAT |
| | GCTGCAGAACTTCACTTGGTTCACTGGAACACCAAATATGGGGATTTTGGGAA |
| | AGCTGTGCAGCAACCTGATGGACTGGCCGTTCTAGGTATTTTTTTGAAGGTTG |
| | GCAGCGCTAAACCGGGCCATCAGAAAGTTGTTGATGTGCTGGATTCCATTAAA |
| | ACAAAGGGCAAGAGTGCTGACTTCACTAACTTCGATCCTCGTGGCCTCCTTCC |
| | TGAATCCCTGGATTACTGGACCTACCCAGGCTCACTGACCACCCCTCCTCTTC |
| | TGGAATGTGTGACCTGGATTGTGCTCAAGGAACCCATCAGCGTCAGCAGCGA |
| | GCAGGTGTTGAAATTCCGTAAACTTAACTTCAATGGGGAGGGTGAACCCGAA |
| | GAACTGATGGTGGACAACTGGCGCCCAGCTCAGCCACTGAAGAACAGGCAAA |
| | TCAAAGCTTCCTTCAAAtaaGCTAGCGTCGACaatcaacctctggattacaaaatttgtgaaagattgac |
| | tggtattcttaactatgttgctccttttacgctatgtggatacgctgctttaatgcctttgtatcatgctattgcttcccgtatggctttcattt |
| | tctcctccttgtataaatcctggttgctgtctctttatgaggagttgtggcccgttgtcaggcaacgtggcgtggtgtgcactgtgtttg |
| | ctgacgcaacccccactggttggggcattgccaccacctgtcagctcctttccgggactttcgctttccccctccctattgccacgg |
| | cggaactcatcgccgcctgccttgcccgctgctggacaggggctcggctgttgggcactgacaattccgtggtgttgtcgggga |
| | agctgacgtcctttccatggctgctcgcctgtgttgccacctggattctgcgcgggacgtccttctgctacgtcccttcggccctcaa |
| | tccagcggaccttccttcccgcggcctgctgccggctctgcggcctcttccgcgtcttcgccttcgccctcagacgagtcggatct |
| | ccctttgggccgcctccccgcctggaattcgagctcggtacctttaagaccaatgacttacaaggcagctgtagatcttagccactt |
| | tttaaaagaaaaggggggactggaagggctaattcactcccaacgaagacaagatctgctttttgcttgtactgggtctctctggtta |
| | gaccagatctgagcctgggagctctctggctaactagggaacccactgcttaagcctcaataaagcttgccttgagtgcttcaagt |
| | agtgtgtgcccgtctgttgtgtgactctggtaactagagatccctcagacccttttagtcagtgtggaaaatctctagcagtagtagtt |
| | catgtcatcttattattcagtatttataacttgcaaagaaatgaatatcagagagtgagaggaacttgtttattgcagcttataatggtta |
| | caaataaagcaatagcatcacaaatttcacaaataaagcatttttttcactgcattctagttgtggtttgtccaaactcatcaatgtatctt |
| | atcatgtctggctctagctatcccgcccctaactccgcccagttccgcccattctccgccccatggctgactaattttttttatttatgca |
| | gaggccgaggccgcctcggcctctgagctattccagaagtagtgaggaggcttttttggaggcctaggcttttgcgtcgagacgt |
| | acccaattcgccctatagtgagtcgtattacgcgcgctcactggccgtcgttttacaacgtcgtgactgggaaaaccctggcgttac |
| | ccaacttaatcgccttgcagcacatccccctttcgccagctggcgtaatagcgaagaggcccgcaccgatcgcccttcccaacag |
| | ttgcgcagcctgaatggcgaatggcgcgacgcgccctgtagcggcgcattaagcgcggcgggtgtggtggttacgcgcagcg |
| | tgaccgctacacttgccagcgccctagcgcccgctcctttcgctttcttcccttcctttctcgccacgttcgccggctttccccgtcaa |
| | gctctaaatcgggggctccctttagggttccgatttagtgctttacggcacctcgaccccaaaaaacttgattagggtgatggttcac |
| | gtagtgggccatcgccctgatagacggtttttcgccctttgacgttggagtccacgttctttaatagtggactcttgttccaaactgga |
| | acaacactcaaccctatctcggtctattcttttgatttataagggattttgccgatttcggcctattggttaaaaaatgagctgatttaaca |
| | aaaatttaacgcgaattttaacaaaatattaacgtttacaatttcccaggtggcacttttcggggaaatgtgcgcggaacccctatttg |
| | tttatttttctaaatacattcaaatatgtatccgctcatgagacaataaccctgataaatgcttcaataatattgaaaaaggaagagtatg |
| | agtattcaacatttccgtgtcgcccttattcccttttttgcggcattttgccttcctgtttttgctcacccagaaacgctggtgaaagtaaa |
| | agatgctgaagatcagttgggtgcacgagtgggttacatcgaactggatctcaacagcggtaagatccttgagagttttcgccccg |
| | aagaacgttttccaatgatgagcacttttaaagttctgctatgtggcgcggtattatcccgtattgacgccgggcaagagcaactcg |
| | gtcgccgcatacactattctcagaatgacttggttgagtactcaccagtcacagaaaagcatcttacggatggcatgacagtaaga |
| | gaattatgcagtgctgccataaccatgagtgataacactgcggccaacttacttctgacaacgatcggaggaccgaaggagctaa |
| | ccgcttttttgcacaacatgggggatcatgtaactcgccttgatcgttgggaaccggagctgaatgaagccataccaaacgacga |
| | gcgtgacaccacgatgcctgtagcaatggcaacaacgttgcgcaaactattaactggcgaactacttactctagcttcccggcaac |
| | aattaatagactggatggaggcggataaagttgcaggaccacttctgcgctcggcccttccggctggctggtttattgctgataaat |
| | ctggagccggtgagcgtgggtctcgcggtatcattgcagcactggggccagatggtaagccctcccgtatcgtagttatctacac |
| | gacggggagtcaggcaactatggatgaacgaaatagacagatcgctgagataggtgcctcactgattaagcattggtaactgtca |
| | gaccaagtttactcatatatactttagattgatttaaaacttcatttttaatttaaaaggatctaggtgaagatcctttttgataatctcatga |
| | ccaaaatcccttaacgtgagttttcgttccactgagcgtcagaccccgtagaaaagatcaaaggatcttcttgagatcctttttttctgc |
| | gcgtaatctgctgcttgcaaacaaaaaaaccaccgctaccagcggtggtttgtttgccggatcaagagctaccaactctttttccga |
| | aggtaactggcttcagcagagcgcagataccaaatactgtccttctagtgtagccgtagttaggccaccacttcaagaactctgta |
| | gcaccgcctacatacctcgctctgctaatcctgttaccagtggctgctgccagtggcgataagtcgtgtcttaccgggttggactca |
| | agacgatagttaccggataaggcgcagcggtcgggctgaacggggggttcgtgcacacagcccagcttggagcgaacgacct |
| | acaccgaactgagatacctacagcgtgagctatgagaaagcgccacgcttcccgaagggagaaaggcggacaggtatccggt |
| | aagcggcagggtcggaacaggagagcgcacgagggagcttccagggggaaacgcctggtatctttatagtcctgtcgggtttc |
| | gccacctctgacttgagcgtcgatttttgtgatgctcgtcaggggggcggagcctatggaaaaacgccagcaacgcggccttttta |
| | cggttcctggccttttgctggccttttgctcacatgttctttcctgcgttatcccctgattctgtggataaccgtattaccgcc |

### Example 9. Rapid Expansion Protocol with Retroviral Transfection of TILs

Pre-REP TILs were prepared similarly to that of Example 1. Briefly, Melanoma and head and neck tumor samples were obtained from Cooperative Human Tissue Network. Tumor samples were cut into 1-3 mm fragments in Hanks' Balanced Salt Solution (HBSS) buffer and fragments were placed in Grex vessels at 1-10 fragments/flask in TIL culture media (RPMI-1640 supplemented with GlutaMAX (Thermo Fisher), 1% Penicillin/Streptomycin, 1 mM Sodium Pyruvate, 1% HEPES, 50 μM 2-Mercaptoethanol (Invitrogen) and 10% heat-inactivated human AB serum (Valley Bio)) containing 6000 IU/mL IL2 (Peprotech), 10 ug·mL 41BB antibody (Creative BioLabs), 30 ng/ml of CD3 antibody (OKT3, Biolegend), and 0.1 mg/mL Normocin (InvivoGen). Vessels were routinely fed when nutrient depletion was identified, roughly every 3-4 days. This culture process is referred to as pre-rapid expansion protocol (pre-REP). After pre-REP, TILs were aliquoted, frozen in cell freezing media (Bambanker, Bulldog Bio or Cryostor-10, STEMCELL Technologies) and stored long-term in liquid nitrogen.

These pre-REP TILs were thawed and rested overnight in TIL media with 6000 IU/mL human IL2. TILs were then activated for 24 hr in 24-well plates coated with OKT3 at 3 μg/mL (Ultra-LEAF purified anti-human CD3 antibody, Biolegend) and 6000 IU/mL human IL2. RetroNectin (30 μg/mL) was used to coat 24-well non-tissue culture cell culture plates overnight at 4° C. The following day, RetroNectin was removed, the plates were blocked with 2% bovine serum albumin (BSA) in PBS, and the plates were then washed with PBS. Gibbon Ape Leukemia Virus (GALV) pseudotyped gamma retroviral vector (where mbIL15-CA2 DRD expression is under control of a promoter derived from murine leukemia virus LTR) supernatants were prepared from a stable producer cell line. Retroviral vector supernatant was diluted in TIL media and added in a total volume of 500 μL per well resulting in an approximate MOI of 16-80. The plates containing viral vector were centrifuged at 1400×g for 2 hr at 32° C., and the supernatant was then removed. After supernatant removal, 1.0×10⁶ activated TILs were transferred per well with 100 IU/mL IL2 and incubated at 37° C. overnight. Cells were processed similarly without virus addition and used as negative control (“unengineered”). 24 hours after transduction, 5×10⁵ TILs were transferred into each well of a 6M GREX well plate (Wilson Wolf) in a total of 60 mL TIL media per well (RPMI-1640 supplemented with GlutaMAX (Thermo Fisher), 1% Penicillin/Streptomycin, 1 mM Sodium Pyruvate, 1% HEPES, 50 μM 2-Mercaptoethanol (Invitrogen) and 10% heat-inactivated human AB serum (Valley Biomed)). Irradiated K562 feeder cells (transduced with 4-1BBL and mbIL21 and irradiated at 100Gy) or irradiated PBMC feeder cells (irradiated at 25Gy) were thawed and added to the culture at a ratio of 50:1 K562:TILs or 200:1 PBMC:TILs, respectively. TILs transduced with the regulated mbIL15 construct received 25 μM Acetazolamide (SelleckChem) and untransduced TILs received 6000 IU/mL IL2. The cells were grown for 14 days in the GREX plates for the “rapid expansion protocol” or REP, and media was added or replaced as necessary.

### Example 10. Regulated mbIL15 Modified TILs: Signaling and Polyfunctionality

ACZ Regulates IL15 Expression and Signaling in Regulated mbIL15 TILs in a Dose-Dependent Fashion.

Pre-REP TILs were prepared similarly to the methods of Example 1-3 and 9, and unengineered and mbIL15 TIL generated accordingly as described in Examples 1-3 and 9. Engagement of the IL15 signaling pathway results in phosphorylation of signal transducers downstream, including the transcription factor protein STAT5 and ribosomal protein S6. To demonstrate that ACZ-regulated mbIL15 expression results in IL15 signaling in regulated mbIL15 TILs, a phospho-flow cytometry-based assay was employed as follows: Cryopreserved regulated mbIL15 TILs obtained from four human donors (Patients 1-4), were thawed and then rest in ACZ-free media for 24 hours. Next, the regulated mbIL15 TILs were regulated for 18 hours in the presence of a range of concentrations of ACZ including 0.1, 1, 2.5, 5, 10, 25, 100 μM, as well as vehicle control. The regulated mbIL15 TILs were then collected for staining and FACS analysis.

Briefly, cells were stained using antibodies for CD3, CD4, CD8, IL15 and a Live/Dead marker. Then cells were fixed in 2% formaldehyde (BD Cytofix) and permeabilized using a methanol-based buffer (BD Phospho Perm III Buffer) before staining with antibodies specific for phosphorylated STAT5 (Biolegend) and S6 (Cell Signaling Technology). Cells were acquired on the BD Symphony and analyzed using FlowJo software.

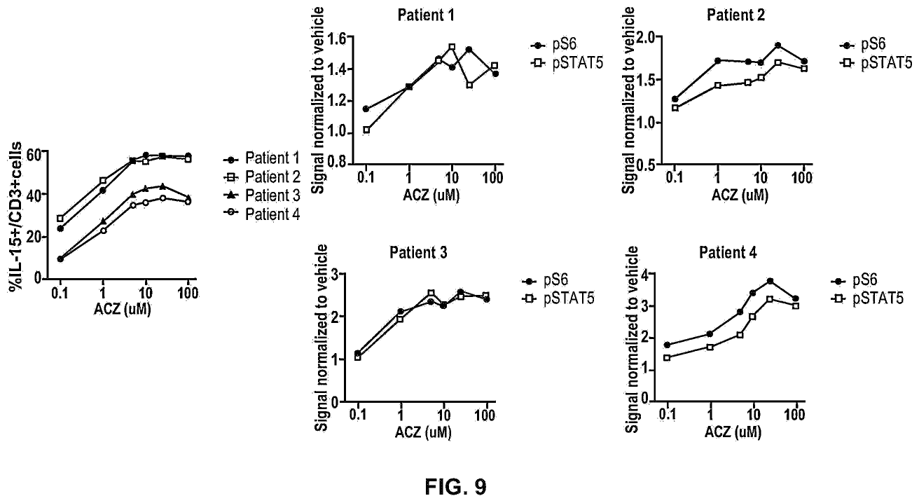

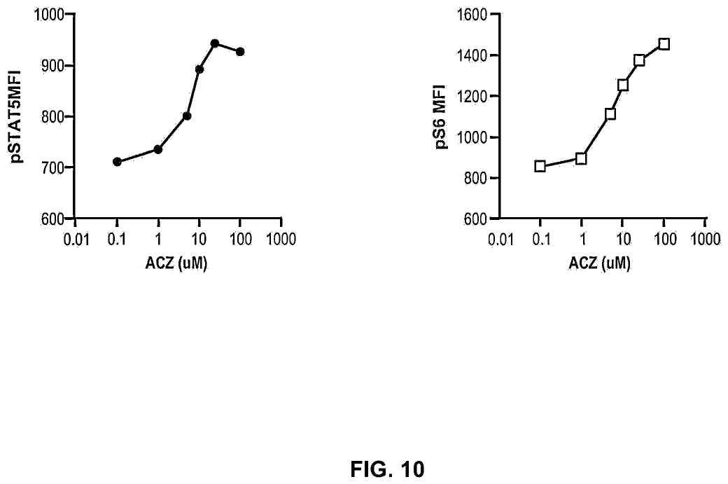

With increasing concentrations of ACZ expression of mbIL15 also increases, plateauing at around 10-25 μM of ACZ. FIG. 9A. Similarly, the staining intensity of pSTAT5 and pS6 increased with higher concentrations of ACZ in regulated mbIL15 TILs, indicative of a greater degree of IL15 signaling. These results show a dose-dependent relationship between ACZ and IL15 expression and signaling. FIGS. 9B-E and FIG. 10.

Constitutive mbIL15 Expression and ACZ Regulation of Regulated mbIL15 TILs Engage the IL15 Signaling Pathway

In order to compare different strategies for IL15 expression, TILs were utilized that constitutively express mbIL15 and regulated mbIL15 TILs. Cryopreserved unengineered TILs, constitutive mbIL15 TILs, and regulated mbIL15 TILs, from three human donors were thawed and then rested in ACZ-free media for 24 hours. Next, the foregoing TILs were regulated in culture media for 18 hours, as follows: (1) 200 IU/mL of IL2 (Peprotech) was added to unengineered TILs; and (2) 25 μM ACZ was added to regulated mbIL15 TIL cultures. Vehicle was added to control conditions. After the 18-hour treatment, the cells were stained using antibodies for CD3, CD4, CD8, IL15 and a Live/Dead marker. Then, cells were fixed in 2% formaldehyde (BD Cytofix) and permeabilized using a methanol-based buffer (BD Phospho Perm III Buffer) before staining with antibodies specific for phosphorylated STAT5 (Biolegend) and S6 (Cell Signaling Technology). Cells were acquired on the BD Fortessa and analyzed using FlowJo software.

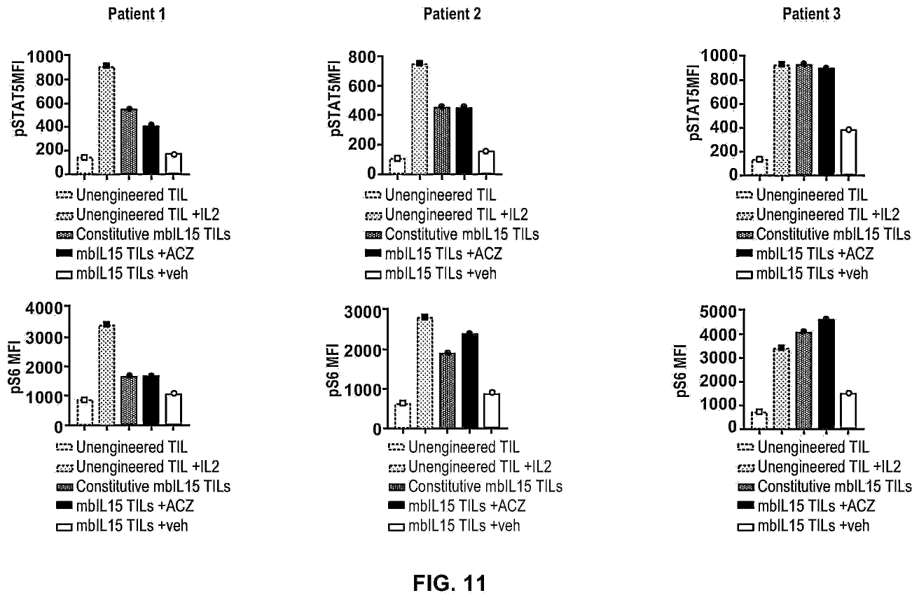

As shown in FIG. 11, IL2 shares an overlapping signaling pathway with IL15, including signaling through STAT5 and S6. Unengineered TILs cultured with IL2 showed increased engagement of the signaling pathway compared to the corresponding vehicle condition. FIG. 11. Similarly, both constitutive mbIL15 expression and regulated mbIL15 TILs+ACZ displayed increased phosphorylation of the STAT5 and S6 compared to the regulated mbIL15 TILs+vehicle control. FIG. 11.

Regulated mbIL15 TILs Demonstrates Greater Polyfunctionality than Unengineered TILs+IL2

Polyfunctional T cells have the capacity to produce multiple effector molecules simultaneously in response to a stimulus. Additionally, polyfunctionality is correlated with T cell efficacy. To compare polyfunctionality of unengineered TILs to regulated mbIL15 TILs, cryopreserved cells were thawed and allowed to rested in IL2- and ACZ-free media for 24 hours. Next, regulation of the cells occurred as follows: unengineered TILs were regulated for 18 hours in the presence of a range of concentrations of IL2 (20, 200, 1000 and 6000 IU/mL, or vehicle); regulated mbIL15 TILs were regulated in the presence of ACZ (0.1, 1, 5, 10, 25, 100 μM ACZ, or vehicle) for 18 hours. Then, cells were stimulated for 6 hours with phorbol 12-myristate 13-acetate (PMA) and ionomycin (Biolegend) in the presence of brefeldin A (Biolegend) and monensin (Life Technologies Corporation). Unstimulated unengineered TILs and unstimulated regulated mbIL15 TILs were used as a control. After stimulation, cells were then collected for staining and FACS analysis.

Briefly, cells were stained using antibodies for CD3, CD4, CD8, IL15 and a viability dye. Then, cells were formaldehyde-fixed and permeabilized (BD Cytofix/Cytoperm kit), then stained using antibodies for TNFα and IFNγ (Biolegend). Cells were acquired on the BD Fortessa and analyzed using FlowJo software. Cells that are double-positive for expression of TNFα and IFNγ are considered polyfunctional.

As shown in FIG. 12, while all culture conditions contained some polyfunctional populations, polyfunctionality in regulated mbIL15 TILs increased with higher concentrations of ACZ. FIG. 12A, 12B. Additionally, regulated mbIL15 TILs were more polyfunctional than unengineered TILs+IL2 from the same donor. FIGS. 12A, 12C. The percent of regulated mbIL15 TILs expressing mbIL15 also displayed a dose-response relationship with ACZ dose.

### Example 11. In Vivo Efficacy of Regulated IL15 TILs

### PDX163A Efficacy

A patient-derived xenograft (PDX) model was created from a fresh primary melanoma sample (Patient tumor No. M1200163A) acquired from a tumor bank (Cooperative Human Tissue Network: CHTN). A mouse model was established using NSG female mice (Jackson Laboratory; Catalog No. 000557). Once the model was established, cryopreserved sections of tumor were aseptically implanted into isoflurane-anesthetized, immune-compromised mice (NSG female mice; Jackson Laboratory; Catalog No. 000557). Tumors were allowed grow to approximately 1000 mm³-2000 mm³ and the mice were then euthanized. The tumors were aseptically collected, sectioned into ˜100 mg sections, and then implanted into a larger cohort of mice that were allowed to grow for 13 days. After 13 days, the tumors were measured and randomized (50 mm³-100 mm³) into respective treatment groups. On the next day, 10 million (10M) TILs were introduced intravenously. TILs were generated according to the rapid expansion protocol (REP) described above.

Treatment groups were as follows: (1) unengineered TILs dosed with IL2; and (2) regulated mbIL15 TILs dosed with acetazolamide (ACZ). Mice receiving unengineered TILs were dosed twice daily with 50,000 International Units (IUs) of IL2 for 5 days. Mice treated with regulated mbIL15 TILs received either vehicle or 200 mg/kg acetazolamide (ACZ) daily, for the entire study. Tumors and body weights were collected twice weekly.

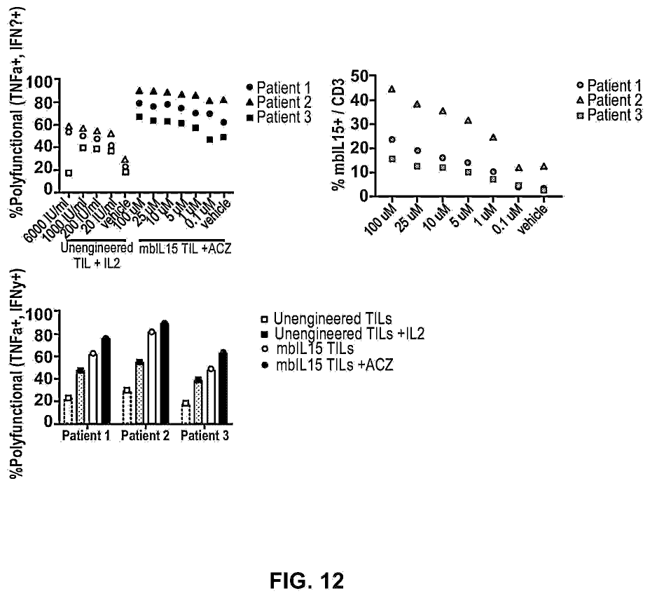

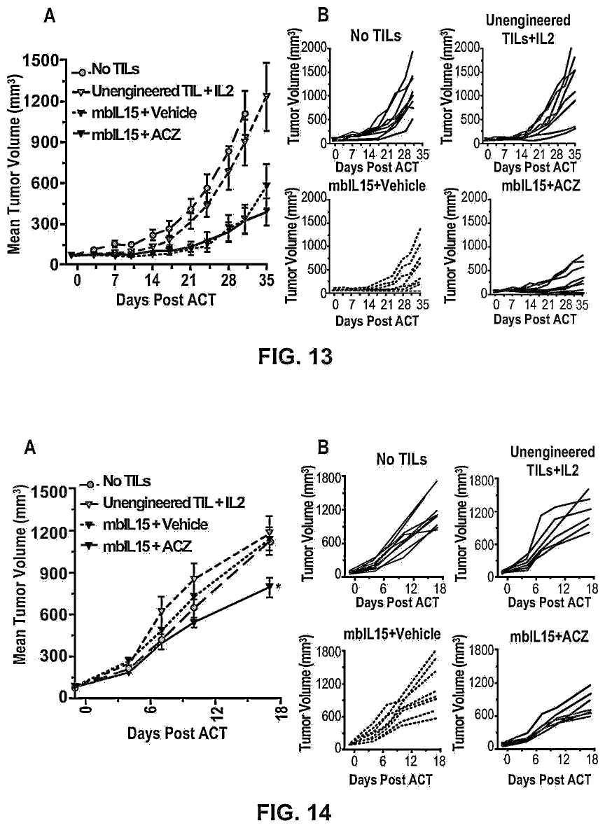

FIG. 13 shows the results of a patient-derived xenograft (PDX) model. At the end of the end of the rapid expansion protocol (REP), unengineered TILs and regulated mbIL15 TILs (+/−acetazolamide (ACZ)) were adoptively transferred into mice bearing a human melanoma PDX. Mean tumor volumes were evaluated (+/−SEM). FIG. 13A shows mean tumor volume for a given treatment at days post adoptive cell transfer (ACT). FIG. 13B shows tumor volume at days post ACT for no TILs (top left); unengineered TILs+IL2 (top right); regulated mbIL15 TILs+vehicle (bottom left); and regulated mbIL15 TILs+ACZ (bottom right). As shown in FIG. 13, regulated mbIL15 TILs+ACZ significantly superior anti-tumor efficacy compared to unengineered TIL+IL2.

### SK-MEL-1 Efficacy

A SK-MEL-1 xenograft cancer model was created to evaluate regulated mbIL15 TILs of the present invention. Cells obtained from the thoracic duct of a patient with widespread and rapidly progressing malignant melanoma (ATCC Catalog No. HTB-67) were used to create the model. NSG female mice (Jackson Laboratory; Catalog No. 000557) were the mice used to receive the cancer cells. Briefly, low passage cells were thawed and grown to scale maintaining viable, sub-confluent cultures. On the day of injection, cells were counted, washed, and resuspended in sterile PBS at a concentration of 30×10⁶ cells/mL (36 cells per injection of 100 μL). Each mouse received 100 μL of cells injected subcutaneously on the shaved right flank using a BD tuberculin syringe, containing a 27 gauge, ½ inch needle. Tumors were allowed to grow for 9 days, and were then measured and randomized (50 mm³-100 mm³) into their respective treatment groups. On the next day, 10 million (10M) TILs were introduced intravenously. TILs were generated according to the rapid expansion protocol (REP) described above.

Treatment groups were as follows: (1) unengineered TILs dosed with IL2; and (2) regulated mbIL15 TILs dosed with acetazolamide (ACZ). Mice receiving unengineered TILs were dosed twice daily with 50,000 International Units (IUs) of IL2 for 5 days. Mice treated with regulated mbIL15 TILs received either vehicle or 200 mg/kg acetazolamide (ACZ) daily for the entire study. Tumors and body weights were collected twice weekly.

FIG. 14 shows the results of a SK-MEL-1 xenograft cancer model. At the end of the end of the rapid expansion protocol (REP), unengineered TILs and regulated mbIL15 TILs (+/−acetazolamide (ACZ)) were adoptively transferred into mice bearing SK-MEL-1 tumors. Mean tumor volumes were evaluated (+/−SEM). FIG. 14A shows mean tumor volume for a given treatment at days post adoptive cell transfer (ACT). FIG. 14B shows tumor volume at days post ACT for no TILs (top left); unengineered TILs+IL2 (top right); regulated mbIL15 TILs+vehicle (bottom left); and regulated mbIL15 TILs+ACZ (bottom right). As shown in FIG. 14, the results demonstrate regulated mbIL15 TILs+ACZ show significantly superior anti-tumor efficacy compared to unengineered TIL+IL2.

### Example 12. In Vitro Cytotoxicity with Regulated mbIL15 TILs

Pre-REP TILs were prepared similarly to the methods of Example 1-3 and 9, and unengineered and mbIL15 TIL generated according to the methods of Examples 1-3 and 9. To evaluate the anti-tumor cytotoxic potential of regulated mbIL15 TILs, a tumor-TIL co-culture assay was performed, using the HLA-matched tumor cell line SK-MEL-1 (ATCC) and six different patient TIL samples. Identical experiments were also set up using PDX cells. The patient TIL samples evaluated were expanded unengineered TILs, or expanded regulated mbIL15 TILs. The regulated mbIL15 TILs were created according to the REP protocol described above (Examples 1-9), and then cryopreserved. Unengineered TILs and regulated mbIL15 TILs from the six patients were then thawed, counted, and rested at a cell density of 7.5×10⁵ cells/mL for 24 hours in culture media supplemented with either: +/−6000 IU/mL IL2 for unengineered TIL, or vehicle (DMSO); or 25 μM ACZ for regulated mbIL15 TILs. The following day, HLA-matched SK-MEL-1 cells were harvested from in vitro culture, and labeled with Cell Trace Far Red, according to the manufacturer's protocol. Additionally, PDX cells were obtained from fresh or cryopreserved chunks and digested with GentleMACs (Miltenyi) according to manufacturer's protocol.

The TILs were then co-cultured at 5:1, and 1:1 (TIL effector:tumor target) ratios with the labeled melanoma cells in the same supplemented IL2 or ACZ conditions listed above, with or without MHC Class I blocking reagent (tumor cells alone cultured with 80 μg/mL of anti-human HLA ABC for 2 hours prior to co-culture with TILs). Additional controls of unlabeled and labeled melanoma cells alone were included to assess background caspase-3 activity in the co-culture system. This TIL-tumor cell co-culture was incubated for 3 hours, after which the cells were fixed, permeabilized, and stained for intracellular cleaved caspase-3 (a marker for irreversible commitment to cell death within tumor cells).

Samples were acquired on the BD Fortessa flow cytometer with analyses conducted using Flow Jo V10.7.1, where cytotoxicity was determined by the percentages of cells staining positive for cleaved caspase-3 within the population of live, Cell Trace Far Red positive cells (subtracting the background caspase-3 positivity).

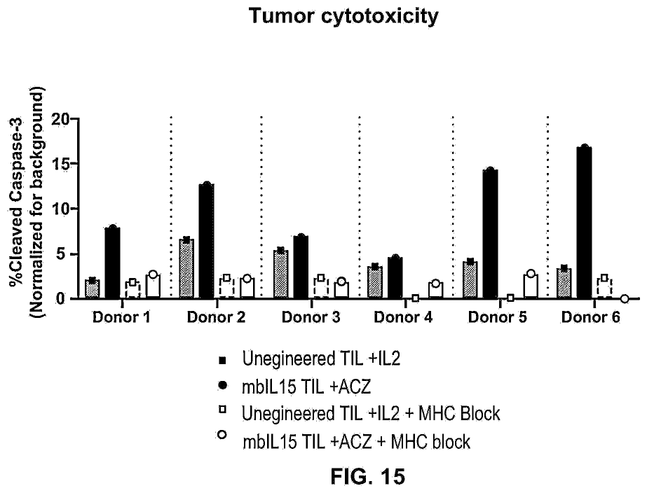

As shown in FIG. 15, in this assessment of anti-tumor cytotoxicity of TIL-tumor pairs, regulated mbIL15 TILs exhibited superior anti-tumor cytotoxic activity across all 6 donors, compared to unengineered TILs+IL2. FIG. 15.

### Example 13: Generation of Unengineered and mbIL15 TIL with Distinct Feeder Cells

Pre-REP TILs generated from tumor samples were prepared as described in Example 1 and 9. Pre-REP TILs were thawed and rested for 48-hours in TIL media (RPMI-1640 supplemented with GlutaMAX (Thermo Fisher), 1% HEPES, 50 M 2-Mercaptoethanol (Invitrogen) and 10% heat-inactivated human AB serum (Valley Bio) with 6000 IU/mL human IL2 (Peprotech). TILs were then activated for 24 hr in 24-well NUNC plates coated with anti-CD3 (OKT3, Miltenyi Biotec) at 3 μg/and 6000 IU/mL soluble human IL2. RetroNectin (30 μg/mL) was used to coat 24-well non-coated cell culture plates overnight at 4° C. The following day, RetroNectin was removed, the plates were blocked with 2.5% human serum albumin (HSA) in PBS, and the plates were then washed with PBS. BaEV-pseudotyped lentiviral supernatants, prepared as described in Example 9, were diluted in TIL media and added to each well to achieve an MOI of 0.01-0.6. The plates containing viral vector were centrifuged at 1400 g for 2 hr at 32° C., and the supernatant was then removed. After supernatant removal, 1×10⁶ activated TILs were transferred per well with 0-100 IU/mL IL2 and incubated at 37° C. overnight. Cells were processed similarly without virus addition into TIL media and used as a negative control (“unengineered”). Twenty-four hours after transduction, TILs were transferred into 6M GREX flasks (Wilson Wolf) into a total of 40 mL TIL REP media (50% TIL media as described above, 50% AIM-V media (Gibco). Proliferation-impaired (irradiated or mitomycin-C treated) feeder cells (pooled PBMCs, unmodified K562 feeders, K562 modified to express membrane-bound IL21, K562 modified to express 41BBL, K562 modified to express 41BBL and membrane-bound IL21) were added to the culture at a ratio of 50:1 K562 to TIL. Groups designated to receive exogenous IL21 were dosed with 50 ng/ml recombinant human IL21. TILs transduced with the regulated mbIL15 construct received 25 μM Acetazolamide (Hikma) and unengineered TILs received 3000 IU/mL IL2. The cells were grown for 14 days in the GREX plates for the “rapid expansion protocol” or REP, and media was added as necessary.

### Evaluation of TIL Expansion in REP

Periodically during the expansion, each GREX well was resuspended and mixed thoroughly, and an aliquot was taken for cell counting using Acridine Orange/Propidium Iodide viability dye (Cellaca Cell Counter, Nexcelom) and flow cytometry staining. Samples were stained using antibodies CD3-BUV395 (BD), CD56-BV711 (Biolegend), CD4-BV605 (Biolegend), CD8-Alexa Fluor 700 (Biolegend), IL15RaFc-Biotin (ACRO Biosystems) with secondary Streptavidin-BV421 (Biolegend), and fixable viability dye eFluor 780 (Thermo Fisher). Samples were run on the BD Symphony flow cytometer and analysis conducted using Flow Jo V10.7.1.

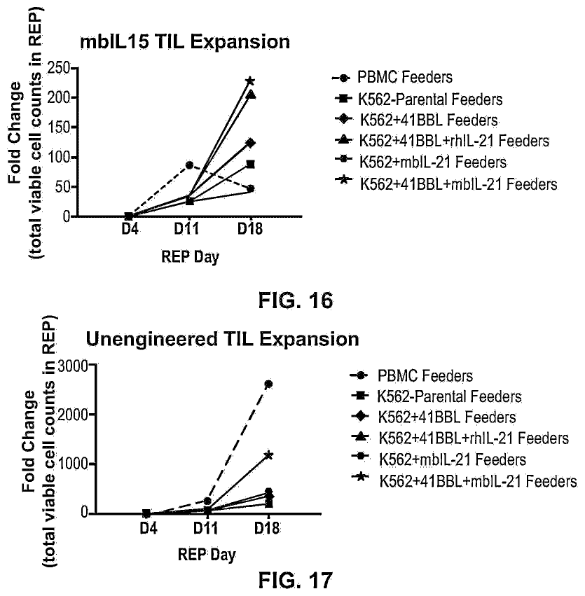

Total TIL expansion was determined by obtaining the total viable cell counts at specific time points throughout REP. FIG. 16 shows that for mbIL15 TILs, use of K562 feeder cells and receiving both IL-21 and 41BBL-mediated co-stimulation resulted in the maximal cell expansion in REP and PBMC feeder cells as well as K562 feeder cells without 41BBL supported only sub-optimal levels of TIL expansion in REP. In contrast, although unengineered TIL expanded in the presence of IL2 using any of the feeder cells, PBMC feeder cells promoted the maximal expansion of unengineereded TIL in REP.

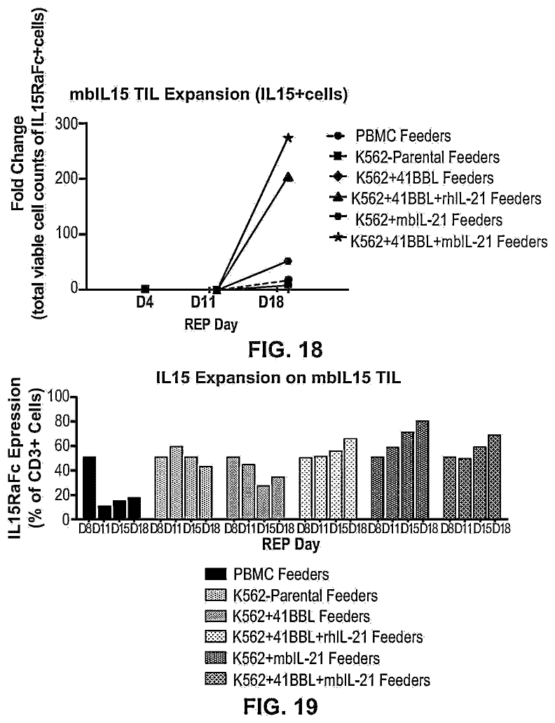

IL15 expression was determined by the percent of cells staining positive for BV421-streptavidin within the population of live, CD3 positive, CD56 negative cells. In mbIL15 TILs generated with K562 feeder cells and receiving both IL-21 and 41BBL-mediated co-stimulation, the frequency of mbIL15+TILs increased through the REP process, suggesting enrichment of the mbIL15-transduced subset within the engineered TIL cell cultures (FIG. 18). Likewise, maximal expansion of mbIL15+TILs in REP occurred when either constitutive or regulated mbIL15+TILs are generated using K562 feeder cells with both IL-21 and 41BBL-mediated co-stimulation (FIG. 19).

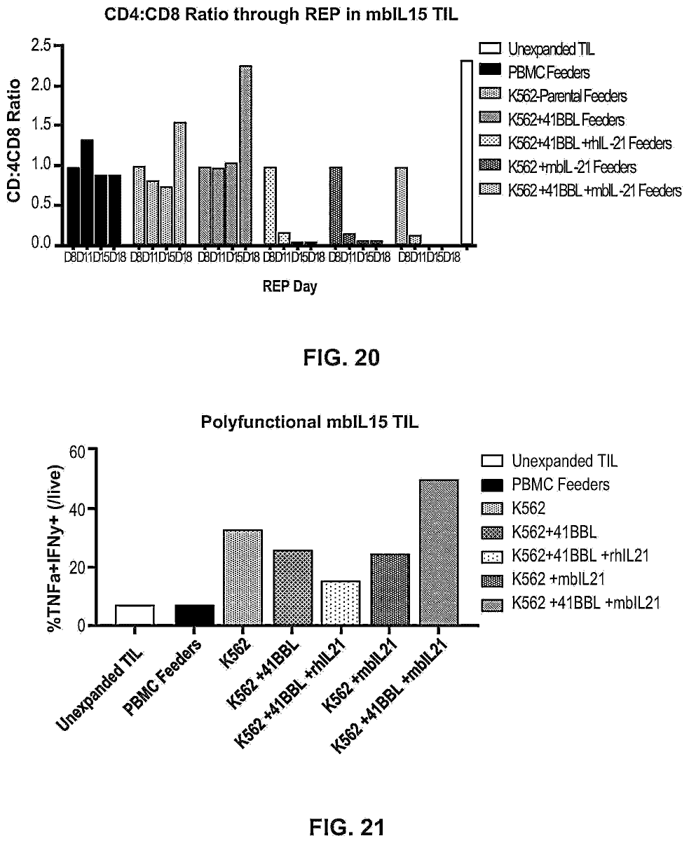

CD4:CD8 ratios were determined by a ratio of the percent of cells staining positive for CD4 (of live, CD3 positive, CD56 negative cells) to the percent of cells staining positive for CD8 (of live, CD3 positive, CD56 negative cells). Expanded mbIL15 TILs generated with K562 feeder cells and receiving both IL-21 and 41BBL-mediated co-stimulation were enriched for CD8+ cytotoxic effector cells, as indicated by their decreased CD4:CD8 ratio throughout REP (FIG. 20). In contrast, the CD4:CD8 ratio of mbIL15 TILs generated with pooled PBMC feeders, unmodified K562 feeders, or K562 feeders expressing 41BBL alone did not decrease during REP.

For evaluation of polyfunctionality, unengineered and mbIL15 TILs at the end of REP were co-cultured in a 96-well tissue culture treated round bottom plate with Immunocult CD3/CD28 stimulation (Stem Cell Technologies) as per manufacturer's protocol. After 1 hour of incubation, 1000× transport inhibitors were added (Monensin from eBiosciences, Brefeldin A from Biolegend), and the co-cultured was incubated at 37° C. for 5 additional hours. After the incubation, samples were stained using the antibodies described above, then fixed and permeabilized using Cytofix/Cytoperm reagents (BD Biosciences). Intracellular staining was performed with antibodies against IL2-BV737 (BD), IFNγ-FITC (Biolegend), Perforin-PerCPCy5.5 (Biolegend), TNFα-PECF594 (Biolegend), granzymeB-Alexa Fluor 700 (Biolegend). Samples were run on the BD Symphony flow cytometer and analysis conducted using Flow Jo V10.7.1. Polyfunctionality was determined as the percent of TNFα and IFNγ double positive cells, of live lymphocytes. mbIL15 TIL generated with K562 feeder cells expressing both membrane-bound IL-21 and 41BBL demonstrated enhanced polyfunctionality at the end of REP as compared to mbIL15 TILs generated with PBMC feeder cells or unmodified K562 feeder cells (FIG. 21).

### Evaluation of In Vitro TIL Persistence in an Antigen-Independent Survival Assay

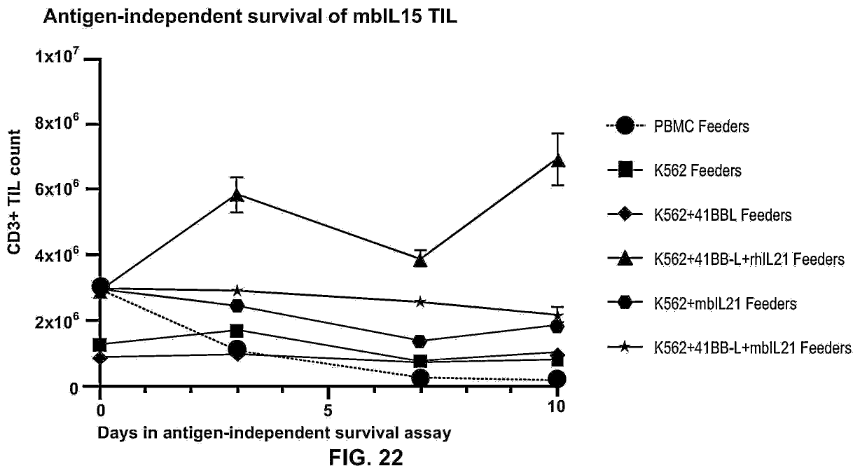

Post-REP TILs were assessed for in vitro persistence in an antigen-independent survival assay. At the end of REP, unengineered and mbIL15 TILs were rested in supplement-free conditions for 24 hours. The following day, unengineered cells were cultured in duplicate at 1×10⁶ cells/well in a 24-well GREX plate either without cytokine support or with 6000 IU/mL IL2, and mbIL15 TILs were cultured at the same density either with 25 μM ACZ or with the identical volume of vehicle (DMSO). On day 0, 100 μL of each well was sampled for TIL enumeration and phenotypic characterization, which was performed by cell count and staining with antibodies as described above. On day 4, cells were resuspended, 500 μL of cells were removed and 500 μL of media+treatment were added to each well to bring the culture volume up to 1000 μL. On day 6, cells were resuspended, a 100 μL aliquot was sampled and phenotyped, 400 μL of cells were removed, and 500 μL of media+treatment were added to each well to bring the culture volume up to 1000 μL. On day 8, cells were resuspended, 500 μL of cells were removed and 500 μL of media+treatment were added to each well to bring the culture volume up to 1000 μL. On day 10, cells were resuspended, a 100 μL aliquot was sampled and phenotyped, and then cultures were terminated. Samples were run on the BD Symphony flow cytometer and analysis conducted using Flow Jo V10.7.1. Expanded mbIL15 TILs generated with K562 feeder cells and receiving both IL-21 and 41BBL-mediated co-stimulation demonstrate improved persistence in a 10-day survival assay compared to mbIL15 TILs generated with PBMC feeder cells or K562 feeder cells that are unmodified or express mbIL-21 and 41BBL independently (FIG. 22).

### Assessment of TCR Diversity

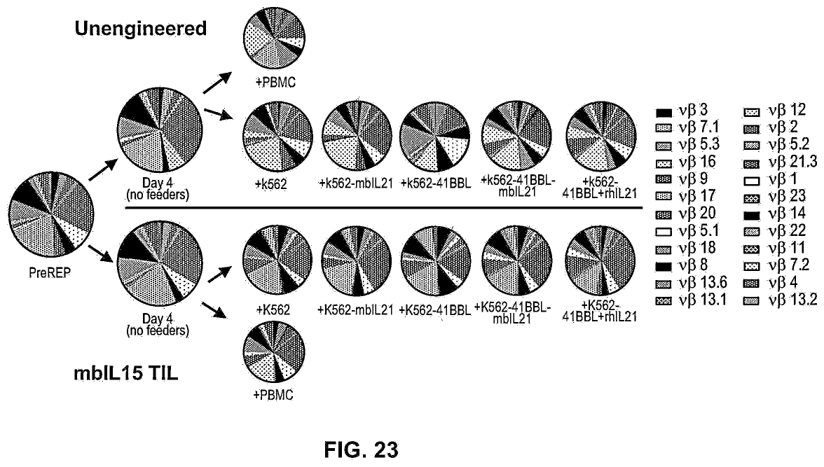

To measure TCRVB sub-family diversity, unengineered and mbIL15 TILs at the end of REP were stained for flow cytometry using the Beta Mark TCR Vbeta Repertoire Kit (Beckman Coulter) following manufacturer's protocol. Samples were run on the BD Symphony flow cytometer and analysis conducted using Flow Jo V10.7.1, and TCRVB subfamily distribution was assessed by evaluating the percent positive for each subfamily and displaying the data as an aggregate of all covered subfamilies. Both unengineered and mbIL15 TILs maintained diverse TCRVB subfamily distribution regardless of the feeder cells for expansion in REP (FIG. 23).

PD1 Expression in mbIL15 TILs with Both 41BBL and IL21-Mediated Signaling

To evaluate the level of TIL exhaustion, PD1 expression was determined. Samples were stained using antibodies CD3-BUV395 (BD), CD56-BV711 (Biolegend), CD4-BV605 (Biolegend), CD8-Alexa Fluor 700 (Biolegend), PD1-PECy7 (Biolegend), CD25-BUV737 (Biolegend), IL15RaFc-Biotin (ACRO Biosystems) with secondary Streptavidin-BV421 (Biolegend), and fixable viability dye eFluor 780 (Thermo Fisher). For intracellular staining, cells were first stained with surface antibodies listed above, and then cells were fixed and permeabilized using BD Cytofix/Cytoperm manufacturer's protocol. Permeabilized cells were then stained using the antibody FoxP3-FITC (Biolegend), and samples were run on the BD Symphony flow cytometer and analysis conducted using Flow Jo V10.7.1. PD1 expression was determined by the percent of cells staining positive for PD1 within the population of live, CD3 positive, CD56 negative cells. As shown in FIG. 25, PD1 expression is highest in unexpanded mbIL15 TIL, and expansion of mbIL15 TILs with both 41BBL and IL21-mediated signaling produces TILs with near baseline expression of PD1.

### Example 14: Phenotype Changes in mbIL15 TILs During Engineering and Expansion as Compared to Pre-REP TILs (Frequencies of CD8+, CD4+, PD1+ and Regulatory T Cells)

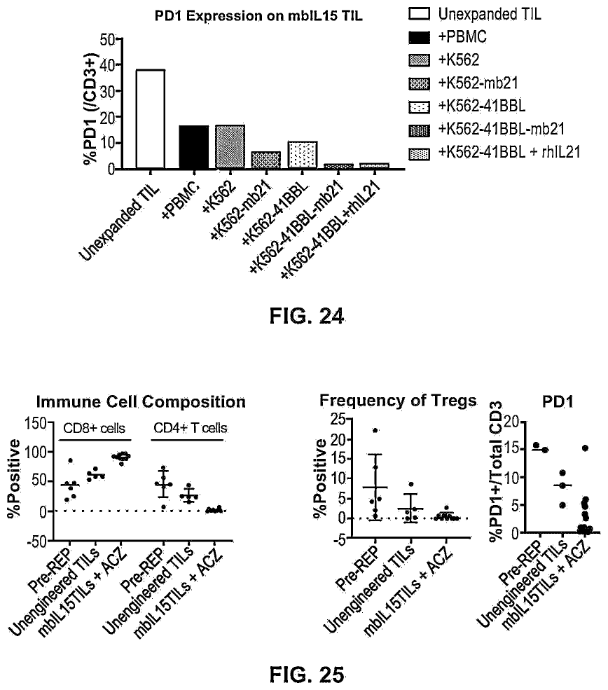

Phenotyping was performed to compare pre-REP TILs (as described in Example 1) to engineered mbIL15 TILs (as described in Example 3). Pre-REP and post-REP TILs were phenotyped by flow cytometry using antibodies for CD3, CD4, CD8, and PD1 as described in Example 13. As shown in FIG. 25A, the frequency of CD8+ T cells is higher and the frequency of CD4+ T cells is lower for post-REP mbIL15 TILs as compared with corresponding pre-REP TILs from the same TIL donors, which is consistent with the results shown in FIG. 20 from Example 13. This increase in CD8+ T cells reflects an increase in cytotoxic effector cells as discussed and evaluated in Example 13. Likewise, as shown in FIG. 25B, the post-REP mbIL15 TILs express lower levels of PD1 than corresponding pre-REP TILs from the same TIL donors, which is consistent with the results shown in FIG. 24 from Example 13.

To detect the regulatory T cells (T_{reg} cells) in the expanded population of TILs, samples were stained using antibodies CD3-BUV395 (BD), CD56-BV711 (Biolegend), CD4-BV605 (Biolegend), CD8-Alexa Fluor 700 (Biolegend), PD1-PECy7 (Biolegend), CD25-BUV737 (Biolegend), IL15RaFc-Biotin (ACRO Biosystems) with secondary Streptavidin-BV421 (Biolegend), and fixable viability dye eFluor 780 (Thermo Fisher). For intracellular staining, cells were first stained with surface antibodies listed above, and then cells were fixed and permeabilized using BD Cytofix/Cytoperm manufacturer's protocol. Permeabilized cells were then stained using the antibody FoxP3-FITC (Biolegend), and samples were run on the BD Fortessa flow cytometer and analysis conducted using Flow Jo V10.7.1. Regulatory T cells were identified as CD3+ T cells that are gated as CD4+ and further classified as CD25 and FoxP3 double positive cells. As shown in FIG. 25C, expanded mbIL15 TILs have a reduced proportion of regulatory T cells as compared to pre-REP TILs prior to the engineering step.

### Example 15: Patient-Derived Xenograft (PDX) Model and Treatment with Engineered TILs Establishment of a Patient-Derived Xenograft (PDX) Model

A patient-derived xenograft (PDX) model (PDX163A) was created from a fresh primary melanoma sample acquired from a tumor bank, as described in Example 11. Once the model was established, cryopreserved sections of tumor were aseptically implanted into isoflurane-anesthetized, immune-compromised mice. Tumors grew to approximately 1000 mm³-2000 mm³ upon when they were euthanized, and tumors were serially passaged into subsequent animals to maintain the PDX tumor growth and build cohorts of animals for efficacy studies (as described below).

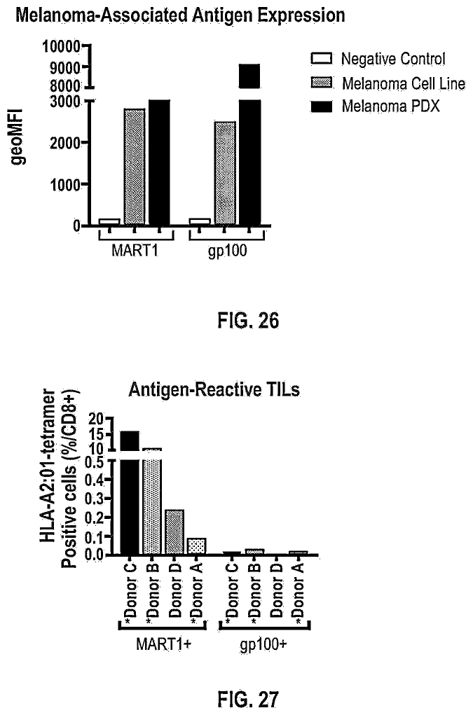

The PDX163A tumors resected from the tumor-bearing animals were also assessed for their expression of shared melanoma tumor antigens using flow cytometry. To evaluate the level of conserved melanoma antigen on melanoma cells, the melanoma cell line A375 and melanoma PDX described herein were assayed by flow cytometry. Tumor chunk(s) from melanoma PDX as described in Example 11 were obtained fresh or from cryopreservation, and were digested with the GentleMACs (Miltenyi) according to manufacturer's protocol in order to obtain a viable single cell suspension Samples were blocked with Fc blocking reagent and stained using antibodies against MART-1 (Biolegend), gp100 (Biolegend) and fixable viability dye eFluor 780 (Thermo Fisher). Samples were run on the BD Symphony flow cytometer and analysis conducted using Flow Jo V10.7.1. The frequency of melanoma-associated antigen-expressing tumor cells was determined by the percent of cells staining positive for either MART-1 or gp 100, within the population of live cells. FIG. 26 shows that the conserved melanoma-associated antigens MART-1 and gp100 were both expressed on the PDX tumors selected for TIL efficacy modeling as described in this Example (below).

### Selection of Donors for Allogeneic Efficacy Modeling

TILs from eight melanoma donors were generated as described in Examples 1-3 or 9. Briefly, after 3 weeks in the pre-REP culture, cryopreserved TILs were thawed and rested overnight with 6000 IU/mL human IL2. TILs were then activated with anti-CD3/CD28 Dynabeads or on OKT3-coated multi-well plates for 24 hours, after which point they were transduced with regulated mbIL15 vectors or unengineered. 24 hours after transduction, TILs were expanded with K562-IL21-41BBL feeder cells in GREX 6M well plates (Wilson Wolf) with 6000 IU/mL IL2 added to unengineered TILs, and 25 μM acetazolamide (SelleckChem or Hikma) added to regulated mbIL15 TILs. After 14 days of expansion, TILs were harvested, de-beaded, and rested overnight with and without IL2 and acetazolamide.

Tetramer staining was used determine which TIL donors were reactive to the shared melanoma antigens, MART-1 and gp100. To evaluate the level of antigen-reactive TILs, flow cytometry was performed to examine the frequency of tetramer-reactive cells. Samples were blocked with Fc blocking reagent and stained using antibodies CD3-BUV395 (BD), CD4-BV605 (Biolegend), CD8-Alexa Fluor 700 (Biolegend), HLA-A2:01-MART-1 tetramer (MBL International), HLA-A2:01-gp100 (MBL International), IL15RaFc-Biotin (ACRO Biosystems) with secondary Streptavidin-BV421 (Biolegend), and fixable viability dye eFluor 780 (Thermo Fisher). Samples were run on the BD Symphony flow cytometer and analysis conducted using Flow Jo V10.7.1. The frequency of antigen-reactive TILs was determined by the percent of cells staining positive for each of the two tetramers, independently, within the population of live, CD3 positive, CD8 positive cells. As shown in Figure X, all four of the donors tested demonstrated reactivity to MART-I antigen, and three of four donors tested demonstrated reactivity to gp 100 antigen. The tetramer positive populations indicate that the TILs contain a portion of cells that are reactive to the corresponding melanoma-associated antigens, through the HLA:A2:01 locus. In FIG. 27, donors indicated with a * were utilized in the PDX efficacy study as depicted in in this Example (below).

Tumor chunk(s) from melanoma PDX as described in Example 11 were obtained fresh or from cryopreservation, and were digested with the GentleMACs (Miltenyi) according to manufacturer's protocol in order to obtain a viable single cell suspension. PDX cells were then resuspended in TIL media at 5×10⁶ cells/mL. Ten μg/mL mitomycin-C was added to the cells, which were then incubated for 30 minutes at 37° C. The cells were then washed three times with 50 mL TIL media. 1×10⁵ PDX cells per well were added to a 96-well flat bottom tissue-culture treated plate. In some wells, 80 μg/mL HLA-ABC (Biolegend) blocking antibody were added to block MHC class I on the target cells. TILs that were rested overnight were added at a 1:1 ratio of TIL:PDX for a total volume of 200 μL per well. As a positive control, TILs were co-cultured 1:1000 with PMA/ionomycin, which would elicit maximal IFNγ secretion. As a negative control, TILs were co-cultured without any additional reagents or cells and identified as “Unstimulated” TIL. At a 24-hour time point, supernatant was saved from each well and the concentration of IFNγ was assayed by MSD.

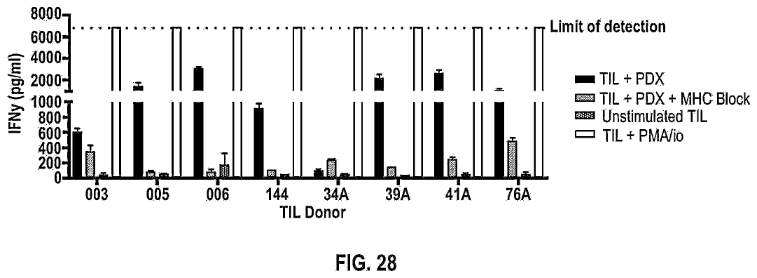

FIG. 28 shows that interferon gamma (IFNγ) production after TIL:tumor cell co-culture can be used to predict TIL donors that are reactive to the PDX tumor. This in vitro assay demonstrates that TIL donors 006, 39A, and 41A are the donors with the highest amount of IFNγ produced in response to the PDX, thus supporting their candidacy as donors to examine in vivo efficacy as described in this Example (below).

### Use of a Patient-Derived Xenograft (PDX) Model for TIL Efficacy Studies

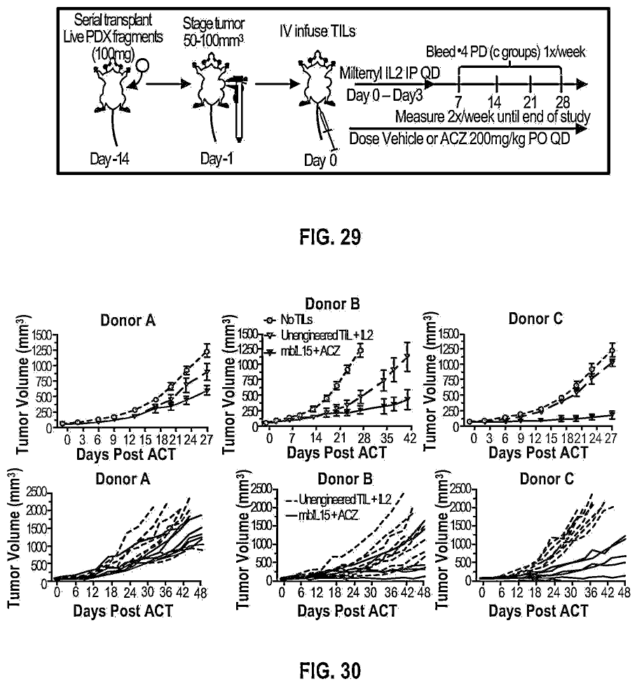

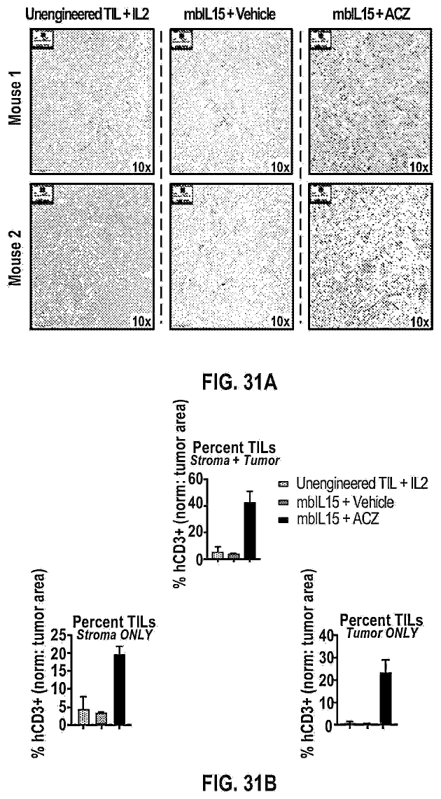

Tumors from PDx-tumor-bearing mice (passaged as described above) were aseptically collected, sectioned into ˜100 mg sections, and then implanted into a larger cohort of mice that were allowed to grow for 13 days upon which being measured and randomized (50 mm³ to 100 mm³) into their respective treatment groups. On the next day, 10M TILs were introduced intravenously. Mice receiving unengineered TILs were dosed daily with 600,000 International units (IUs) IL2 for 4 days. Mice receiving the mbIL15 product in which mbIL15 was operably linked to CA2 received 200 mg/kg acetazolamide (ACZ) daily for the entire study. Tumors and body weights were collected twice weekly. The treatment paradigm is shown in FIG. 29. As shown in FIG. 30, the engineered TILs+ACZ showed superior anti-tumor effects as compared to unengineered TILs+IL2. Additionally, the engineered TILs, particularly in the presence of ACZ, showed better tumor infiltration as shown in FIG. 31A and greater numbers in both stroma and tumor compartments as shown in FIG. 31B.

1. A method for treating cancer in a recipient subject, comprising administering to the recipient subject a population of allogeneic tumor-infiltrating lymphocytes (TILs).

2. The method of claim 1, wherein the TILs are administered to the recipient subject without the need for IL2 administration.

3. The method of claim 1, wherein the allogeneic TILs are modified to express a membrane bound IL15.

4. The method of claim 2, wherein the membrane bound IL15 is operably linked to a drug responsive domain (DRD) and wherein the DRD is responsive to a ligand.

5. The method of claim 4, further comprising administering to the recipient subject the ligand.

6. The method of claim 1, further comprising selecting the allogeneic TILs from a donor who is HLA-matched to the recipient subject.

7. The method of claim 1, wherein the recipient subject is lymphodepleted prior to administration of the allogeneic TILs.

8. The method of claim 6, wherein the recipient subject and donor are human.

9. The method of claim 1, further comprising
(a) determining tumor antigen expression in a tumor of the recipient subject and
(b) screening a population of TILs derived from a donor for T-cell receptors (TCRs) specific for one or more antigens expressed in the tumor of the recipient subject to identify a TIL population having a TCR-matched TIL population.

10. The method of claim 1, further comprising assessing the efficacy of the allogeneic TILs comprising determining the cytotoxic effect of a TIL population on a tumor cell line, wherein the tumor cell line is HLA-matched with the recipient subject and wherein the tumor cell line expresses one or more antigens expressed in the tumor of the recipient subject.

11. The method of claim 10, wherein the cytotoxic effect is determined by measuring the induction of caspase 3 cleavage in the tumor cells.

12. The method of claim 1, further comprising assessing the efficacy of the allogeneic TILs comprising
(a) implanting tumor cells or fragments from the recipient subject into an immunodeficient or humanized starter animal under conditions for growth of a starter tumor;
(b) implanting tumor cells or tumor fragments from the starter tumor into an immunodeficient or humanized test animal to create a test tumor;
(c) administering to the test animal the allogeneic TILs; and
(d) evaluating the allogeneic TIL efficacy in treating the test tumor.

13. The method of claim 10, wherein TIL efficacy is evaluated by measuring the size of the test tumor before and after administration of the TILs, by detecting TIL infiltration into the test tumor after administration, or by imaging of the test animal.

14. The method of claim 10, wherein the test animal and the starter animal are mice.

15. The method of claim 14, wherein the mice are humanized mice, athymic nude mice, severely compromised immune deficient (SCID) mice, NOD-SCID mice, or recombination-activating gene 2 (Rag2)-knockout mice.

16. The method of claim 10, wherein the modified TILs are administered to the test animal without the need for IL2.

17. The method of claim 1, further comprising deriving allogeneic TILs from a donor; modifying the derived, allogeneic TILs to express a membrane bound IL15; and expanding the modified TILs in vitro.

18. The method of claim 17, wherein the expansion step comprises expanding the modified TILs in the presence of K562 feeder cells, wherein the K562 feeder cells are modified to express 41BB ligand (41BBL) and IL21.

19. The method of claim 18, wherein the IL21 is membrane bound.

20. The method of claim 18, wherein the modified TILs are expanded without the need for IL2.

21. The method of claim 1, further comprising isolating a tumor sample or tumor cells from the recipient subject.

22. A method for assessing the efficacy of HLA-matched, allogeneic TILs in treating cancer in a recipient subject comprising
(a) implanting tumor cells or tumor fragments from the recipient subject in an immunodeficient or humanized starter animal under conditions for growth of a starter tumor;
(c) implanting tumor cells or tumor fragments from the starter tumor in an immunodeficient or humanized test animal to create a test tumor;
(d) administering to the test animal the HLA-matched allogeneic TILs; and
(e) evaluating the allogeneic TIL efficacy in treating the test tumor.

23. The method of claim 22, wherein TIL efficacy is evaluated by measuring the size of the test tumor before and after administration of the TILs, by detecting TIL infiltration into the test tumor after administration, or by imaging of the test animal.

24. The method of claim 22, wherein the test animal and the starter animal are mice.

25. The method of claim 24, wherein the mice are humanized mice, athymic nude mice, severely compromised immune deficient (SCID) mice, NOD-SCID mice, or recombination-activating gene 2 (Rag2)-knockout mice.

26. The method of claim 22, further comprising deriving allogeneic TILs from a donor; modifying the derived allogeneic TILs to express a membrane bound IL15; and expanding the modified TILs in vitro.

27. The method of claim 26, wherein the expansion step comprises expanding the modified TILs in the presence of K562 feeder cells, wherein the K562 feeder cells are modified to express 41BB ligand (41BBL) and IL21.

28. The method of claim 27, wherein the IL21 is membrane bound.

29. The method of claim 27, wherein the modified TILs are expanded without the need for IL2.

30. The method of claim 22, wherein the modified TILs are administered to the test animal without the need for IL2.

31. The method of claim 7, wherein the lymphodepletion comprises administering to the subject cyclophosphamide.

32. The method of claim 7, wherein the lymphodepletion comprises administering to the subject 50 to 300 mg/m²/day cyclophosphamide.

33. The method of claim 31, wherein the cyclophosphamide is administered to the subject for 2 to 7 days prior to TIL administration.

34. The method of claim 31, wherein the cyclophosphamide is administered to the subject for 2 to 4 days prior to TIL administration.

35. The method of claim 7, wherein the lymphodepletion comprises administering to the subject cyclophosphamide and fludarabine.

36. The method of claim 35, wherein the cyclophosphamide and fludarabine is administered to the subject for 2 to 7 days prior to TIL administration.

37. The method of claim 35, wherein the cyclophosphamide and fludarabine is administered to the subject for 2 to 4 days prior to TIL administration.


SEQUENCE LISTING (60 sequences)

SEQ ID NO: 1 - protein, 159 aa, synthetic construct [sequence omitted]

SEQ ID NO: 2 - protein, 187 aa, Homo sapiens
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 3 - DNA, 3919 bp, Homo sapiens
  source 1..3919: mol_type = other DNA
agtcccagacagaacctactatgtgcggcggcagctggggcgggaaggcgggagctgggggcgctgggggcgctgcggccgctgcggccgctgcagccgctgcagcgccagggtccacctggtcggctgcacctgtggaggaggaggtggatttcaggcttcccgtagactggaagaatcggctcaaaaccgcttgcctcgcaggggctgagctggaggcagcgaggccgcccgacgcaggcttccggcgagacatggcagggcaaggatggcagcccggcggcagggcctggcgaggagcgcgagcccgcggccgcagttcccaggcgtctgcgggcgcgagcacgccgcgaccctgcgtgcgccggggcgggggggcggggcctcgcctgcacaaatggggacgaggggggcggggcggccacaatttcgcgccaaacttgaccgcgcgttctgctgtaacgagcgggctcggaggtcctcccgctgctgtcatggttggttcgctaaactgcatcgtcgctgtgtcccagaacatgggcatcggcaagaacggggacctgccctggccaccgctcaggaatgaattcagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgattaatatgaaggtgttttctagtttaagttgttccccctccctctgaaaaaagtatgtatttttacattagaaaaggttttttgttgactttagatctataattatttctaagcaactagtttttattccccactactcttgtctctatcagataccatttatgagacattcttgctataactaagtgcttctccaagaccccaactgagtccccagcacctgctacagtgagctgccattccacacccatcacatgtggcactcttgccagtccttgacattgtcgggcttttcacatgttggtaatatttattaaagatgaagatccacatacccttcaactgagcagtttcactagtggaaataccaaaagcttcctacgtgtatatccagaggtttgtagataaatgttgccaccttgtttgtaacagtgaaaaattgaaaacaacctggaagtccagtgatgggaaaatgagtatgtttctgtcttagattggggaacccaaagcagattgcaagactgaaatttcagtgaaagcagtgtatttgctaggtcataccagaaatcatcaattgaggtacggagaaactgaactgagaaggtaagaaaagcaatttaaagtcagcgagcaggttctcattgataacaagctccatactgctgagatacagggaaatggaggggggaaagctggagtattgatcccgcccccctccttggttgtcagctccctgtcctgtgtgtgggcggaacatagtccagctgctctatagcaagtctcaggtgtttgcagtaagaagctgctggcatgcacgggaacagtgaatgccaaacacttaaagcaattcgatgtttaagtatgtaagttcttttttttttagacagcgtttcgctcttgttgcccaggctagcatgcaatggtgtgacctcggcttactgcaacctccgccttcccagattcaagcgattctcctgcctcaggctcccaagtagctaggaccaggtgcgcgccaccacgcccggctaatttttgtattttgtatttttagtagagatggggtttcaccatgttggtcaggctagtctcgaactcgtgaccgcaagcgattcacccacctcagcctcccaaagtgctgggattaccggcttgagccaccacacccggcacatcttcattctttttatgtagtaaaaagtataaggccacacatggtttatttgaagtattttataatttaaaaaaatacagaagcaggaaaaccaattataagttcaagtgagggatgatggttgcttgaaccaaagggttgcatgtagtaagaaattgtgatttaagatatattttaaagttataagtagcaggatattctgatggagtttgactttggttttgggcccagggagtttcagatgcctttgagaaatgaatgaagtagagagaaaataaaagaaaaaccagccaggcacagtggctcacacctgtaatcccagcgctttgggaggctaaggcaggcagatcacttgagaccagcttgggcaacatggcaaagccccatctctacaaaaaacacaaaaattagctgggcattgtggcgcacacctgtattcccatctagtcaggaagctgagatggaagaattaattgagcccacgagttcaaggctgcagtgagtcgtgattgtgccactgcactccagccggggtgacagaagagaccttgtctcgaaaaggaatctgaaaacaatggaaccatgccttcataattctagaaagttattttcaactgataaatctatattcacccaaataatcaagggtgaaggtaaaataatacatttttagacaagcaaagactcaggggttacctccatgtgccctttttagggaagctgttggagaaaatactccagcaaaatgaaggagtacacaaaccagagaatgacatgaatccagcaaataggatccaacacaggcaatattccagctatggagctagctttaaaaaggaacagtaaaaatattaatcggttagctgggtggaatggcccatgcctgtagtcccagctactcaggaggctcagcagcaggacgacttgagcccaagagttccagaccagcctggccaccttagtgagatcccttctcttaaaaataataacttattgccagatttggggcatttggaaagaagttcattgaagataaagcaaaagtaaaaaaaaaaaaaaaaaaaacaaggggaaagggttggttaggcaatcattctagggcagaaagaagtacaggataggaagagcataatacactgtttttctcaacaaggagcagtatgtacacagtcataatgatgtgactgcttagcccctaaatatggtaactactctgggacaatatgggaggaaaagtgaagattgtgatggtgtaagagctaaatcctcatctgtcatatccagaaatcactatataatatataataatgaaatgactaagttatgtgaggaaaaaaacagaagacattgctaaaagagttaaaagtcattgctctggagaattaggagggatggggcaggggactgttaggatgcattataaactgaaaagcctttttaaaattttatgtattaatatatgcattcacttgaaaaactaaaaaaaaacaataatttggaaaaacccatgaaggtaactaacggaaggaaaaactaagagaatgaaaagtatttgcctctggaaagaacaactggcaggactgttgttttcattgtaagacttttggagccatttaattgtacttaaccattttcatctatttctttaataagaacaattccatcttaataaagagttacacttgttaataagt

SEQ ID NO: 4 - protein, 107 aa, Homo sapiens
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 5 - protein, 327 aa, Homo sapiens
MEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 6 - protein, 875 aa, Homo sapiens
MERAGPSFGQQRQQQQPQQQKQQQRDQDSVEAWLDDHWDFTFSYFVRKATREMVNAWFAERVHTIPVCKEGIRGHTESCSCPLQQSPRADNSAPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 7 - protein, 260 aa, Homo sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 8 - DNA, 1492 bp, Homo sapiens
  source 1..1492: mol_type = other DNA
acacagtgcaggcgcccaagccgccgccgccagatcggtgccgattcctgccctgccccgaccgccagcgcgaccatgtcccatcactgggggtacggcaaacacaacggacctgagcactggcataaggacttccccatcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccttggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaataagatggtcccatagtctgtatccaaataatgaatcttcgggtgtttccctttagctaagcacagatctaccttggtgatttggaccctggttgctttgtgtctagttttctagacccttcatctcttacttgatagacttactaataaaatgtgaagactagaccaattgtcatgcttgacacaactgctgtggctggttggtgctttgtttatggtagtagtttttctgtaacacagaatataggataagaaataagaataaagtaccttgactttgttcacagcatgtagggtgatgagcactcacaattgttgactaaaatgctgcttttaaaacataggaaagtagaatggttgagtgcaaatccatagcacaagataaattgagctagttaaggcaaatcaggtaaaatagtcatgattctatgtaatgtaaaccagaaaaaataaatgttcatgatttcaagatgttatattaaagaaaaactttaaaaattattatatatttatagcaaagttatcttaaatatgaattctgttgtaatttaatgacttttgaattacagagatataaatgaagtattatctgtaaaaattgttataattagagttgtgatacagagtatatttccattcagacaatatatcataacttaataaatattgtattttagatatattctctaataaaattcagaattcta

SEQ ID NO: 9 - protein, 595 aa, Homo sapiens [sequence omitted]

SEQ ID NO: 10 - protein, 22 aa, synthetic construct
MDMRVPAQLLGLLLLWLSGARC

SEQ ID NO: 11 - DNA, 66 bp, synthetic construct
  source 1..66: mol_type = other DNA
atggacatgcgggtgcctgcacaacttctgggcctgctgttgttgtggctgtctggagcccggtgt

SEQ ID NO: 12 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 13 - DNA, 342 bp, Homo sapiens
  source 1..342: mol_type = other DNA
aattgggtaaatgttatcagtgatctcaagaagatagaggatctcatccagtccatgcatattgatgccacgctgtacacagaaagcgatgtgcatcctagctgtaaggtgacagcgatgaagtgttttcttttggagctgcaggtaattagtcttgagtccggcgatgccagcattcatgataccgtagaaaacttgattatcctggccaacaattctctgtcctcaaacggaaacgtaaccgagagcggttgtaaagaatgtgaagaactggaagaaaagaacatcaaggagtttctgcaatcattcgttcacatcgtacaaatgttcataaatacgtca

SEQ ID NO: 14 - protein, 30 aa, synthetic construct
GSGSGSGSGSGSGSGSGSGSGSGSGSGSGS

SEQ ID NO: 15 - DNA, 90 bp, synthetic construct
  source 1..90: mol_type = other DNA
ggatctggttctggttccggaagtggatctggttcagggtccggtagtggatctgggtcaggaagtggaagcggtagtgggtctggatct

SEQ ID NO: 16 - protein, 8 aa, synthetic construct
KQEHFPDN

SEQ ID NO: 17 - DNA, 24 bp, synthetic construct
  source 1..24: mol_type = other DNA
aaacaagagcactttcctgataac

SEQ ID NO: 18 - protein, 21 aa, synthetic construct
LLPSWAITLISVNGIFVICCL

SEQ ID NO: 19 - DNA, 63 bp, synthetic construct
  source 1..63: mol_type = other DNA
ctgttgccgagctgggcgattacgcttatcagtgtaaacggcatctttgtaatatgctgtctg

SEQ ID NO: 20 - protein, 25 aa, synthetic construct
TYCFAPRCRERRRNERLRRESVRPV

SEQ ID NO: 21 - DNA, 75 bp, synthetic construct
  source 1..75: mol_type = other DNA
acctactgcttcgcaccaaggtgccgggagagaaggagaaatgaaagactgagaagggagagcgtgagacctgtg

SEQ ID NO: 22 - protein, 25 aa, synthetic construct
TYCFAPRCRERARNERLRRETVRPV

SEQ ID NO: 23 - DNA, 75 bp, synthetic construct
  source 1..75: mol_type = other DNA
acctactgcttcgcaccaaggtgccgggagagagcaagaaatgaaagactgagaagggagaccgtgagacctgtg

SEQ ID NO: 24-25 - skipped (2 entries)

SEQ ID NO: 26 - protein, 208 aa, synthetic construct
SHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 27 - DNA, 777 bp, synthetic construct
  source 1..777: mol_type = other DNA
tcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgagaatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 28 - protein, 222 aa, synthetic construct
MDMRVPAQLLGLLLLWLSGARCNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSGSGSGSGSGSGSGSGSGSGSGSGSGSKQEHFPDNLLPSWAITLISVNGIFVICCLTYCFAPRCRERRRNERLRRESVRPVGS

SEQ ID NO: 29 - DNA, 666 bp, synthetic construct
  source 1..666: mol_type = other DNA
atggacatgcgggtgcctgcacaacttctgggcctgctgttgttgtggctgtctggagcccggtgtaattgggtaaatgttatcagtgatctcaagaagatagaggatctcatccagtccatgcatattgatgccacgctgtacacagaaagcgatgtgcatcctagctgtaaggtgacagcgatgaagtgttttcttttggagctgcaggtaattagtcttgagtccggcgatgccagcattcatgataccgtagaaaacttgattatcctggccaacaattctctgtcctcaaacggaaacgtaaccgagagcggttgtaaagaatgtgaagaactggaagaaaagaacatcaaggagtttctgcaatcattcgttcacatcgtacaaatgttcataaatacgtcaggatctggttctggttccggaagtggatctggttcagggtccggtagtggatctgggtcaggaagtggaagcggtagtgggtctggatctaaacaagagcactttcctgataacctgttgccgagctgggcgattacgcttatcagtgtaaacggcatctttgtaatatgctgtctgacctactgcttcgcaccaaggtgccgggagagaaggagaaatgaaagactgagaagggagagcgtgagacctgtgggatcc

SEQ ID NO: 30 - protein, 430 aa, synthetic construct
MDMRVPAQLLGLLLLWLSGARCNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSGSGSGSGSGSGSGSGSGSGSGSGSGSKQEHFPDNLLPSWAITLISVNGIFVICCLTYCFAPRCRERRRNERLRRESVRPVGSSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 31 - DNA, 1443 bp, synthetic construct
  source 1..1443: mol_type = other DNA
atggacatgcgggtgcctgcacaacttctgggcctgctgttgttgtggctgtctggagcccggtgtaattgggtaaatgttatcagtgatctcaagaagatagaggatctcatccagtccatgcatattgatgccacgctgtacacagaaagcgatgtgcatcctagctgtaaggtgacagcgatgaagtgttttcttttggagctgcaggtaattagtcttgagtccggcgatgccagcattcatgataccgtagaaaacttgattatcctggccaacaattctctgtcctcaaacggaaacgtaaccgagagcggttgtaaagaatgtgaagaactggaagaaaagaacatcaaggagtttctgcaatcattcgttcacatcgtacaaatgttcataaatacgtcaggatctggttctggttccggaagtggatctggttcagggtccggtagtggatctgggtcaggaagtggaagcggtagtgggtctggatctaaacaagagcactttcctgataacctgttgccgagctgggcgattacgcttatcagtgtaaacggcatctttgtaatatgctgtctgacctactgcttcgcaccaaggtgccgggagagaaggagaaatgaaagactgagaagggagagcgtgagacctgtgggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgagaatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaa

SEQ ID NO: 32 - DNA, 51 bp, synthetic construct
  source 1..51: mol_type = other DNA
atgtggctgcagtctctgctcctcttggggactgtcgcctgttctatttca

SEQ ID NO: 33 - protein, 17 aa, synthetic construct
MWLQSLLLLGTVACSIS

SEQ ID NO: 34 - DNA, 399 bp, synthetic construct
  source 1..399: mol_type = other DNA
caaggacaggatcgacatatgattcggatgcgccaactgatagatatagtcgatcaactcaagaattatgtgaatgacttggtccctgagtttctgccggctccagaggacgttgaaacaaactgtgaatggtcagcgttttcatgttttcaaaaggcacagctcaagtccgccaatacaggcaataacgagcggattataaatgtctcaattaaaaagctcaagcgcaaacccccttcaacgaatgctggtcgccgccagaaacacaggttgacctgtccctcctgtgactcatacgagaagaaacctcccaaggaatttctcgaacgctttaagtcactcttgcagaagatgattcatcagcacttgagtagccggacacatggttcagaggatagt

SEQ ID NO: 35 - protein, 133 aa, synthetic construct [sequence omitted]

SEQ ID NO: 36 - DNA, 36 bp, synthetic construct
  source 1..36: mol_type = other DNA
gagtctaagtatggcccaccgtgtcccccctgccca

SEQ ID NO: 37 - protein, 12 aa, synthetic construct
ESKYGPPCPPCP

SEQ ID NO: 38 - DNA, 651 bp, synthetic construct
  source 1..651: mol_type = other DNA
gcacctgagttcctcggaggcccctctgtattcctgtttcccccaaagcccaaggatactcttatgatctcacgcactccggaagtaacctgcgtggtggtggatgtgagtcaggaagaccccgaagtccagtttaattggtacgtggacggggttgaggtacataacgccaaaacgaaacctcgggaggagcaattcaattccacttaccgggttgtatcagtcctgactgtactgcatcaagattggctcaacgggaaagagtacaagtgtaaggttagtaataaagggctgccgtctagtattgagaaaacgatcagtaaggctaaagggcagccaagagagccacaagtatataccctgccaccctctcaggaggagatgactaaaaaccaagtgtcactgacctgccttgttaagggtttttacccatctgatatagcagtagagtgggaatccaatggacagccagagaacaattataagactacacctcccgtccttgatagtgacggctccttcttcttgtattctcgacttacagttgataagtcccgctggcaggagggtaatgtctttagctgcagtgtaatgcacgaagctcttcataatcactacacacaaaaatcattgagcctgtctctgggaaag

SEQ ID NO: 39 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - DNA, 66 bp, synthetic construct
  source 1..66: mol_type = other DNA
atggccttgattgtgctcggcggagttgcaggcctgctcctttttattggactcggaatatttttc

SEQ ID NO: 41 - protein, 22 aa, synthetic construct
MALIVLGGVAGLLLFIGLGIFF

SEQ ID NO: 42-43 - skipped (2 entries)

SEQ ID NO: 44 - DNA, 57 bp, synthetic construct
  source 1..57: mol_type = other DNA
gctactaacttcagcctgctgaagcaggctggagacgtggaggagaaccctggacct

SEQ ID NO: 45 - protein, 19 aa, synthetic construct
ATNFSLLKQAGDVEENPGP

SEQ ID NO: 46 - DNA, 762 bp, synthetic construct
  source 1..762: mol_type = other DNA
atggagtacgctagtgatgcgtccttggaccccgaggcgccatggccaccggccccgcgagctcgagcctgtcgagtgctgccatgggctctggtcgctgggttgctcctccttctgcttttggccgcggcttgtgcagtgtttcttgcttgcccgtgggcagttagcggtgctcgcgcatctcccggaagcgcggcgagtcctcgactcagggaaggtccggagctgagcccagatgaccccgccggtttgctggacctccgccaaggaatgttcgctcaactcgttgcgcaaaacgtacttcttatagacggccctcttagttggtacagtgacccaggattggctggcgttagtttgacaggcggactcagttacaaggaggatactaaggaactggtagtcgctaaggctggggtatactacgtgttctttcaactcgaactgagaagggtggttgcgggagaaggatctggaagtgtatctctcgccctgcacctccaacccctcagaagtgccgccggagcggccgcccttgcccttactgtcgacctgcccccggcttcttcagaagcgcgaaatagtgcattcggcttccaggggcgccttttgcacttgagcgctggacagcgcctcggggtccacctccacacggaagcgcgggcgaggcacgcttggcaactcacacaaggtgcgacggttctcggcttgtttagggttacgcctgagataccggctggcctcccatctccaagatccgag

SEQ ID NO: 47 - protein, 254 aa, synthetic construct
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 48 - skipped

SEQ ID NO: 49 - DNA, 2040 bp, synthetic construct
  source 1..2040: mol_type = other DNA
atgtggctgcagtctctgctcctcttggggactgtcgcctgttctatttcacaaggacaggatcgacatatgattcggatgcgccaactgatagatatagtcgatcaactcaagaattatgtgaatgacttggtccctgagtttctgccggctccagaggacgttgaaacaaactgtgaatggtcagcgttttcatgttttcaaaaggcacagctcaagtccgccaatacaggcaataacgagcggattataaatgtctcaattaaaaagctcaagcgcaaacccccttcaacgaatgctggtcgccgccagaaacacaggttgacctgtccctcctgtgactcatacgagaagaaacctcccaaggaatttctcgaacgctttaagtcactcttgcagaagatgattcatcagcacttgagtagccggacacatggttcagaggatagtgagtctaagtatggcccaccgtgtcccccctgcccagcacctgagttcctcggaggcccctctgtattcctgtttcccccaaagcccaaggatactcttatgatctcacgcactccggaagtaacctgcgtggtggtggatgtgagtcaggaagaccccgaagtccagtttaattggtacgtggacggggttgaggtacataacgccaaaacgaaacctcgggaggagcaattcaattccacttaccgggttgtatcagtcctgactgtactgcatcaagattggctcaacgggaaagagtacaagtgtaaggttagtaataaagggctgccgtctagtattgagaaaacgatcagtaaggctaaagggcagccaagagagccacaagtatataccctgccaccctctcaggaggagatgactaaaaaccaagtgtcactgacctgccttgttaagggtttttacccatctgatatagcagtagagtgggaatccaatggacagccagagaacaattataagactacacctcccgtccttgatagtgacggctccttcttcttgtattctcgacttacagttgataagtcccgctggcaggagggtaatgtctttagctgcagtgtaatgcacgaagctcttcataatcactacacacaaaaatcattgagcctgtctctgggaaagatggccttgattgtgctcggcggagttgcaggcctgctcctttttattggactcggaatatttttcggatctggagctactaacttcagcctgctgaagcaggctggagacgtggaggagaaccctggacctatggagtacgctagtgatgcgtccttggaccccgaggcgccatggccaccggccccgcgagctcgagcctgtcgagtgctgccatgggctctggtcgctgggttgctcctccttctgcttttggccgcggcttgtgcagtgtttcttgcttgcccgtgggcagttagcggtgctcgcgcatctcccggaagcgcggcgagtcctcgactcagggaaggtccggagctgagcccagatgaccccgccggtttgctggacctccgccaaggaatgttcgctcaactcgttgcgcaaaacgtacttcttatagacggccctcttagttggtacagtgacccaggattggctggcgttagtttgacaggcggactcagttacaaggaggatactaaggaactggtagtcgctaaggctggggtatactacgtgttctttcaactcgaactgagaagggtggttgcgggagaaggatctggaagtgtatctctcgccctgcacctccaacccctcagaagtgccgccggagcggccgcccttgcccttactgtcgacctgcccccggcttcttcagaagcgcgaaatagtgcattcggcttccaggggcgccttttgcacttgagcgctggacagcgcctcggggtccacctccacacggaagcgcgggcgaggcacgcttggcaactcacacaaggtgcgacggttctcggcttgtttagggttacgcctgagataccggctggcctcccatctccaagatccgagggatcctaa

SEQ ID NO: 50 - protein, 679 aa, synthetic construct
MWLQSLLLLGTVACSISQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFGSGATNFSLLKQAGDVEENPGPMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGS

SEQ ID NO: 51 - DNA, 9772 bp, synthetic construct
  source 1..9772: mol_type = other DNA
gcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcattgatcacgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatcactagtaccatgtggctgcagtctctgctcctcttggggactgtcgcctgttctatttcacaaggacaggatcgacatatgattcggatgcgccaactgatagatatagtcgatcaactcaagaattatgtgaatgacttggtccctgagtttctgccggctccagaggacgttgaaacaaactgtgaatggtcagcgttttcatgttttcaaaaggcacagctcaagtccgccaatacaggcaataacgagcggattataaatgtctcaattaaaaagctcaagcgcaaacccccttcaacgaatgctggtcgccgccagaaacacaggttgacctgtccctcctgtgactcatacgagaagaaacctcccaaggaatttctcgaacgctttaagtcactcttgcagaagatgattcatcagcacttgagtagccggacacatggttcagaggatagtgagtctaagtatggcccaccgtgtcccccctgcccagcacctgagttcctcggaggcccctctgtattcctgtttcccccaaagcccaaggatactcttatgatctcacgcactccggaagtaacctgcgtggtggtggatgtgagtcaggaagaccccgaagtccagtttaattggtacgtggacggggttgaggtacataacgccaaaacgaaacctcgggaggagcaattcaattccacttaccgggttgtatcagtcctgactgtactgcatcaagattggctcaacgggaaagagtacaagtgtaaggttagtaataaagggctgccgtctagtattgagaaaacgatcagtaaggctaaagggcagccaagagagccacaagtatataccctgccaccctctcaggaggagatgactaaaaaccaagtgtcactgacctgccttgttaagggtttttacccatctgatatagcagtagagtgggaatccaatggacagccagagaacaattataagactacacctcccgtccttgatagtgacggctccttcttcttgtattctcgacttacagttgataagtcccgctggcaggagggtaatgtctttagctgcagtgtaatgcacgaagctcttcataatcactacacacaaaaatcattgagcctgtctctgggaaagatggccttgattgtgctcggcggagttgcaggcctgctcctttttattggactcggaatatttttcggatctggagctactaacttcagcctgctgaagcaggctggagacgtggaggagaaccctggacctatggagtacgctagtgatgcgtccttggaccccgaggcgccatggccaccggccccgcgagctcgagcctgtcgagtgctgccatgggctctggtcgctgggttgctcctccttctgcttttggccgcggcttgtgcagtgtttcttgcttgcccgtgggcagttagcggtgctcgcgcatctcccggaagcgcggcgagtcctcgactcagggaaggtccggagctgagcccagatgaccccgccggtttgctggacctccgccaaggaatgttcgctcaactcgttgcgcaaaacgtacttcttatagacggccctcttagttggtacagtgacccaggattggctggcgttagtttgacaggcggactcagttacaaggaggatactaaggaactggtagtcgctaaggctggggtatactacgtgttctttcaactcgaactgagaagggtggttgcgggagaaggatctggaagtgtatctctcgccctgcacctccaacccctcagaagtgccgccggagcggccgcccttgcccttactgtcgacctgcccccggcttcttcagaagcgcgaaatagtgcattcggcttccaggggcgccttttgcacttgagcgctggacagcgcctcggggtccacctccacacggaagcgcgggcgaggcacgcttggcaactcacacaaggtgcgacggttctcggcttgtttagggttacgcctgagataccggctggcctcccatctccaagatccgagggatcctaaatcgggctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattac

SEQ ID NO: 52 - DNA, 8436 bp, synthetic construct
  source 1..8436: mol_type = other DNA
gcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcattgatcacgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattac

SEQ ID NO: 53 - DNA, 8384 bp, synthetic construct
  source 1..8384: mol_type = other DNA
tttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagactagtaccatggacatgcgggtgcctgcacaacttctgggcctgctgttgttgtggctgtctggagcccggtgtaattgggtaaatgttatcagtgatctcaagaagatagaggatctcatccagtccatgcatattgatgccacgctgtacacagaaagcgatgtgcatcctagctgtaaggtgacagcgatgaagtgttttcttttggagctgcaggtaattagtcttgagtccggcgatgccagcattcatgataccgtagaaaacttgattatcctggccaacaattctctgtcctcaaacggaaacgtaaccgagagcggttgtaaagaatgtgaagaactggaagaaaagaacatcaaggagtttctgcaatcattcgttcacatcgtacaaatgttcataaatacgtcaggatctggttctggttccggaagtggatctggttcagggtccggtagtggatctgggtcaggaagtggaagcggtagtgggtctggatctaaacaagagcactttcctgataacctgttgccgagctgggcgattacgcttatcagtgtaaacggcatctttgtaatatgctgtctgacctactgcttcgcaccaaggtgccgggagagaaggagaaatgaaagactgagaagggagagcgtgagacctgtgggatcctaagctagcgtcggcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 54 - DNA, 9161 bp, synthetic construct
  source 1..9161: mol_type = other DNA
tttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagactagtaccatggacatgcgggtgcctgcacaacttctgggcctgctgttgttgtggctgtctggagcccggtgtaattgggtaaatgttatcagtgatctcaagaagatagaggatctcatccagtccatgcatattgatgccacgctgtacacagaaagcgatgtgcatcctagctgtaaggtgacagcgatgaagtgttttcttttggagctgcaggtaattagtcttgagtccggcgatgccagcattcatgataccgtagaaaacttgattatcctggccaacaattctctgtcctcaaacggaaacgtaaccgagagcggttgtaaagaatgtgaagaactggaagaaaagaacatcaaggagtttctgcaatcattcgttcacatcgtacaaatgttcataaatacgtcaggatctggttctggttccggaagtggatctggttcagggtccggtagtggatctgggtcaggaagtggaagcggtagtgggtctggatctaaacaagagcactttcctgataacctgttgccgagctgggcgattacgcttatcagtgtaaacggcatctttgtaatatgctgtctgacctactgcttcgcaccaaggtgccgggagagaaggagaaatgaaagactgagaagggagagcgtgagacctgtgggatcctcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgagaatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccatcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaataagctagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 55 - protein, 259 aa, synthetic construct
SHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 56 - protein, 260 aa, synthetic construct
MSHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 57 - protein, 259 aa, synthetic construct
SHHWGYGKHNGPEHWHKDFPIAKGERQSPVDIDTHTAKYDPSLKPLSVSYDQATNLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 58 - protein, 18 aa, synthetic construct
MDWTWILFLVAAATRVHS

SEQ ID NO: 59 - protein, 48 aa, synthetic construct
MRISKPHLRSISIQCYLCLLLNSHFLTEAGIHVFILGCFSAGLPKTEA

SEQ ID NO: 60 - protein, 33 aa, synthetic construct
MGLVRRGARAGPRMPRGWTALCLLSLLPSGFMA